(19) United States
(12) Patent Application Publication
(10) Pub. No.: US 2006/0281091 A1
(43) Pub. Date: Dec. 14, 2006

(54) Genes regulated in ovarian cancer a s prognostic and therapeutic targets

(76) Inventor: Christian Nicolas Lavedan, Potomac, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/562,406

(22) PCT Filed: Jul. 1, 2004
(86) PCT No.: PCT/EP04/07167

§ 371 Date: Jun. 15, 2006

Related U.S. Application Data
(60) Provisional application No. 60/484,665, filed on Jul. 7, 2003.

(51) Int. Cl. C12Q 1/68; G01N 33/574

(52) U.S. Cl. 435/6; 435/7.23

(57) ABSTRACT

This invention relates to the use of genomic analysis to detect the presence of ovarian cancer in a patient from a sample of tissue or blood and to kits for carrying out this determination. In addition this invention relates to methods to treat a patient with ovarian cancer.

### TECHNICAL FIELD

The present invention belongs to the fields of medicine and relates to the use of genomic analysis to evaluate and treat ovarian cancer. In particular, this invention relates to the measurement of patterns of gene expression to determine the presence of ovarian cancer in a patients tissues.

### BACKGROUND ART

Ovarian cancer is one of the most common types of cancer that affects women in the United States, with a lifetime risk of approximately 1/70. See Whittemore, Gynecol. Oncol., Vol. 55, No. 3, Part 2, pp. S15-S19 (1994). It is a rapidly fatal disease usually detected late, with still no good method of prevention. The greatest risk factor for ovarian cancer is a family history of the disease, suggesting the strong influence of genetics. See Schildkraut and Thompson, Am. J. Epidemiol., Vol. 128, No. 3, pp. 456-466 (1988). Other factors such as demographic, lifestyle and reproductive factors have also been shown to contribute to the risk of ovarian cancer.

Several microarray expression analyses of ovarian biopsies and cell lines have been conducted to identify genes specifically over-expressed in ovarian cancers. See Schummer et al., Gene, Vol. 238, No. 2, pp. 375-385 (1999). Other studies have tried to correlate gene expression levels with specific tumor types. See Bayani et al., Cancer Res., Vol. 62, No. 12, pp. 3466-3476 (2002); Welsh et al., Proc. Natl. Acad. Sci. USA, Vol. 98, No. 3, pp. 1176-1181 (2001); and Ono et al., Cancer Res., Vol. 60, No. 18, pp. 5007-5011 (2000).

These kinds of studies, aimed at Increasing our understanding of the molecular mechanism of tumor development and in some cases at better classifying tumors, has provided a list of genes with few overlaps between analyses. Some technical differences may explain in part the apparent lack of consistency or low reproducibility between studies: quality of samples, amplification of the messenger ribonucleic acid (mRNA) and different microarray platforms. However, it is likely that the heterogeneity of the tumors is a key factor that contributes to the differences observed between studies, in particular, those where few tumors are analyzed. Furthermore, comparison of gene expression levels on microarray experiments have historically been done using ratios of signal intensity (fold change), with limited use of statistical methods and a lack of validation with additional samples.

However, genes apparently expressed at high levels, or with the biggest change in expression, may not always be the most relevant; it is conceivable that a small disruption of the very tight regulation of genes may have dramatic consequences, even when the level of expression is low.

Thus there is a need for the identification of genes whose expression rates are consistently and reliably altered in ovarian cancer. Such a list could provide new insight into ovarian tumor development and progression, and suggest potential new drug targets, and biomarkers for diagnosis, monitoring and treatment of the disease.

### DISCLOSURE OF INVENTION

In the present invention, the application of a combination of statistical tests and the recently described leave-one-out method [see van't Veer et al., Nature, Vol. 415, No. 6871, pp. 530-536 (2002)], allows the analyze of expression profiles of tumors and normal ovarian tissues and for these patterns to also be determined in gene expression products in various body fluids including, but not limited to, blood and serum. See van't Veer et al., supra.

The study of two independent sets of samples, a test set and a validation set, confirms the involvement of several known genes with ovarian tumor development, but also identify novel genes. These findings provide new insight into ovarian tumor development and progression, and suggest potential new drug targets, and biomarkers for diagnosis and monitoring of the disease.

In one embodiment, this invention provides a method to determine if a patient is afflicted with ovarian cancer comprising:
a) obtaining a sample from the said patient;
b) determining the levels of gene expression of two or more of the genes listed in Table 9 in the sample from the patient;
c) comparing the levels of gene expression of the two or more genes determined in (b) to the levels of the same genes listed in Table 1;
d) determining the degree of similarity (DOS) between the levels of gene expression of the two or more genes determined in (c); and
e) determining from the DOS between the level of gene expression of the two or more genes the probability that the sample shows evidence of the presence of ovarian cancer in the patient.

In a preferred embodiment, this invention provides a method wherein the levels of gene expression are determined for a subset of the genes listed in Table 9 comprising genes Nos. 1-28 in Table 9.

In another embodiment, the invention employs a sample comprising cells obtained from the patient. These may be cells removed from a solid tumor in the said patient or, in a preferred embodiment, the sample comprises blood cells and serum drawn from the said patient. In a most preferred embodiment, the sample comprises a body fluid drawn from the patient.

In a preferred embodiment, this invention employs a method of determining the level of gene expression comprising measuring the levels of protein expression product in the sample from the patient. This may be done in a variety of ways including, but not limited to, detecting the presence and level of the protein expression products using a reagent which specifically binds with the proteins, wherein the reagent may be selected from the group consisting of an antibody, an antibody derivative and an antibody fragment.

In another embodiment, this invention provides a method wherein the levels of expression in the sample are assessed by measuring the levels in the sample of the transcribed polynucleotides of the two or more gene in Table 9. These transcribed polynucleotide may be mRNA or complementary DNA (cDNA).

In a preferred embodiment, this method would further include the step of amplifying the transcribed polynucleotide.

In another embodiment, this invention includes a method of treating a subject afflicted with ovarian cancer, the method comprising providing to cells of the subject an antisense oligonuceotide complimentary to one or more of the genes whose expression is up-regulated in ovarian cancer as shown in Table 8.

In addition, this invention provides a method of inhibiting ovarian cancer in a subject at risk for developing ovarian cancer, the method comprising inhibiting expression of one or more of the genes shown in Table 8 to be up-regulated in ovarian cancer.

This invention also provides kits for use in determining treatment strategy for a patient with suspected ovarian cancer comprising:
a) a number (for example, two or more) of antibodies able to recognize and bind to the polypeptide expression product of the two or more of the genes in Table 9;
b) a container suitable for containing the said antibodies and a sample of body fluid from the said individual wherein the antibody can contact the polypeptide expressed by the two or more genes shown in Table 9 if they are present;
c) means to detect the combination of the said antibodies with the polypeptides expressed by the two or more genes shown in Table 9; and
d) instructions for use and interpretation of the kit results.

In another embodiment, this invention provides a kit for use in determining the presence or absence of ovarian cancer in a patient comprising:
a) a number (for example, two or more) of polynucleotides able to recognize and bind to the mRNA expression product of the two or more genes shown in Table 9;
b) a container suitable for containing the said polynucleotides and a sample of body fluid from the said individual wherein the said polynucleotide can contact the mRNA, if it is present;
c) means to detect the levels of combination of the said polynucleotide with the mRNA from the two or more genes shown in Table 9; and
d) instructions for use and interpretation of the kit results.

### BRIEF DESCRIPTION OF DRAWINGS

FIG. 1(A). Re-Classification of Samples Using Increasing Number of Probe Sets.

FIG. 1(B). Plot of Errors as a Function of Number of Probe Sets for Determination of Optimum Number of Classification Genes. Calculated values by increasing number of individual probe sets from the top 6 to the top 55 (A) or to all 900 (B). Arrow indicates the minimum number of probe sets (N=28) that minimizes misclassification.

FIG. 2. Determination of a Threshold CC Value for Classification of Ovarian Status.

FIG. 3. Correlation of Test and Validation Biopsy Profiles with Mean Normal Profile for Different Size Probe Sets. N or T represent Normal or Tumor status, respectively. “r” is the PCC value of the probe set profile of the corresponding biopsy sample with the mean Normal profile (Group 1). Samples are ordered from highest CC to lowest.

FIG. 4. Correlation of Biopsy Profiles with Mean of All Normal Profiles for Different Size Probe Sets. N or T represent Normal or Tumor status, respectively. “r” is the PCC value of the probe set profile of the corresponding biopsy sample with the mean profile of all Normal samples. Samples are ordered from highest CC to lowest.

### MODES FOR CARRYING OUT THE INVENTION

The present invention provides methods to determine whether or not a sample from a patient including, but not limited to, biopsy tissue or blood, serum or some other body fluid from a patient, contains evidence of the presence of ovarian cancer in the patient.

This invention is based, in part, on the discovery of approximately 900 genes which are differentially expressed in tissue from ovarian cancer as compared to normal tissue. This methods of this invention comprise measuring the activities of the approximately 900 or fewer genes that are shown to be differently-expressed in ovarian cancer as compared to normal tissue.

In a preferred embodiment, only a small fraction of the 900 genes would be measured. These measurements, could, in various embodiments, be in the tissue itself from biopsies, etc., or in preferred embodiments could be performed as more indirect measurement of gene expression including, but not limited to, cRNA or polypeptide expression products in various tissues including blood or other body fluids.

The measurements, direct or indirect, of the rates of expression of two or more of these 900 genes from an individual whose tissues status was unknown could then be compared to the expression values for the same two or more genes measured in ovarian cancer tissue or normal tissue.

The “degree of similarity” (DOS) of the unknown two or more gene expression values to the cancer tissue versus normal tissue would then be determined.

This DOS could be determined by any procedure that produces a result whose value is a known function of the DOS between the two groups of numbers, i.e., the measured gene expression values of the two or more genes in tissue from an individual whose ovarian cancer status is unknown and to be determined and the measured gene expression values for the same two or more genes from individuals whose tissue is known to contain ovarian cancer and from individuals whose tissue is known not to contain ovarian cancer.

As used herein the term “DOS” shall mean the extent to which the pattern of gene expression values are alike or numerically similar, as measured by a comparison of the values of gene expression determined by direct or indirect methods.

In a preferred embodiment, the DOS would be determined by a mathematical calculation resulting in a correlation coefficient (CC). In a particularly preferred embodiment, the Pearson Correlation Coefficient (PCC) would be determined but any other mathematical procedure that produces a result whose value is a known function of the DOS between the two groups of numbers could be used.

The value of the DOS (PCC), so calculated, can then be directly related to the probability that the tissue sample is from a patient who does or does not have ovarian cancer. That is to say, the higher the patients' DOS (CC or PCC) as compared to the gene expression values from a patient who does not have ovarian cancer or the higher the DOS (CC or PCC) as compared to the gene expression values from a patient who does have ovarian cancer then the greater the probability that the patient does not or does have ovarian cancer, respectively.

Thus, in a given case, the value of the DOS can be used to determine probabilities for the presence of ovarian cancer. Those of skill in the art will understand that the clinical circumstance for each patient will dictate the value of the DOS (PCC) to be used as a cutoff or to help make clinical decisions with regard to a specific patient. For example, in one embodiment, it is desirable to determine with optimal accuracy the number of a group of patients who have ovarian cancer. This means to minimize both false positives (No Ovarian Cancer misclassified as Ovarian Cancer) and at the same time to minimize false negatives (Ovarian Cancer misclassified as No Ovarian Cancer).

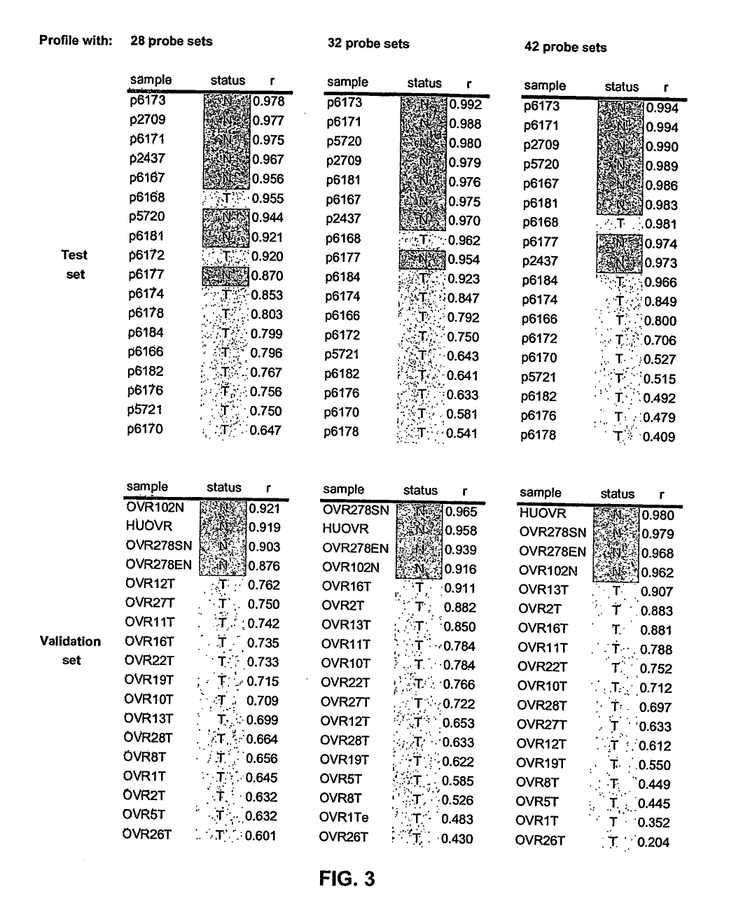

In one preferred embodiment of the present invention, this would work as shown in FIG. 3, using the 28 predictor probe set (as described below) if the gene expression profile correlates with the mean normal (No Ovarian Cancer) profile with a CC≦0.920 the tissue sample is 63 times more likely to contain ovarian cancer then if the CC>0.920 [odds ratio (OR)=63 with 95% confidence interval (CI): 3.3-1194.7].

To use this threshold in one embodiment of this invention, a patient whose gene expression profile when compared with the mean No Ovarian Cancer expression profile achieves a PCC of >0.920 would be classified in the No Ovarian Cancer group and would be presumed not to have ovarian cancer, while a patient whose expression profile was had a PCC of ≦0.920 would be classified in the Ovarian Cancer group and would be assumed to have ovarian cancer with a high probability.

In a further preferred embodiment, the PCC can be set to produce optional sensitivity. That is, to make the smallest possible number of false negatives (Ovarian Cancer misclassified as No Ovarian Cancer). Such an optimal sensitivity setting would be indicated in situations where the occurrence of ovarian cancer must be ruled out with the greatest certainty obtainable. In this embodiment, the threshold is determined by setting the PCC to >0.955. In this case, in the example given, using the 28 predictor probes shown in Table 9 (probe sets 1-28 shown in Table 9), 100% of patients with a CC of >0.955 as compared to the No Ovarian Cancer group did not have ovarian cancer and 100% of the patients whose CC were <0.870, as compared to the No Ovarian Cancer group, did have ovarian cancer.

As is shown in the example, one of skill in the art can choose a PCC that will either maximize sensitivity or maximize specificity or produce any desired ratio of false positives or false negatives. One of skill in the art can easily adjust their choice of PCC to the clinical situation to provide maximum benefit and safety to the patient.

Another aspect of the of the invention are methods to treat ovarian cancer. These methods consist of various efforts to suppress the excess gene expression of the genes that have been found to be up-regulated in ovarian cancer. These genes are shown in Table 8. Methods to decrease the excess expression of these gene would include, but not be limited to, use of antisense DNA, siRNA and methods to complex and deactivate the protein expression products of these over-expressed genes.

Methods of Measurement

In some embodiments of this invention, the gene expression of a selected group of the 900 genes is determined by measuring mRNA levels from tissue samples as described below.

In some embodiments, the gene expression can be measured more indirectly by measuring polypeptide gene expression products in tissues including, but not limited to, tumor and blood tissue.

In some embodiments, gene expression is measured by identifying the presence or amount of one or more proteins encoded by one of the genes listed in Table 9.

The present invention also provides systems for detecting two or more markers of interest, e.g., two or more markers from Table 2. For example, where it is determined that a finite set of particular markers provides relevant information, a detection system is provided that detects the finite set of markers. For example, as opposed to detecting all genes expressed in a tissue with a generic microarray, a defined microarray or other detection technology is employed to detect the plurality, e.g., 28, 42, etc., of markers that define a biological condition, e.g., the presence or absence of ovarian cancer, etc.

The present invention is not limited by the method in which gene expression biomarkers are detected or measured. In some embodiments, mRNA, cDNA or protein is detected in tissue samples, e.g., biopsy samples. In other embodiments, mRNA, cDNA or protein is detected in bodily fluids, e.g., serum, plasma, urine or saliva. A preferred embodiment of the invention provides that the method of the invention is performed ex vivo. The present invention further provides kits for the detection of these relevant gene expression biomarkers.

In some preferred embodiments, protein or the polypeptide expression product is detected. Protein expression may be detected by any suitable method. In some embodiments, proteins are detected by binding of an antibody specific for the protein. For example, in some embodiments, antibody binding is detected using a suitable technique including, but not limited to, radioimmunoassay, enzyme-linked immunosorbant assay (ELISA), “sandwich” immunoassays, immunoradiometric assays, gel diffusion precipitation reactions, immunodiffusion assays, in situ immunoassays, e.g., using colloidal gold, enzyme or radioisotope labels, e.g., Western blots, precipitation reactions, agglutination assays, e.g., gel agglutination assays, hemagglutination assays, etc., complement fixation assays, immunofluorescence assays, protein A assays, immunoelectrophoresis assays and proteomic assays, such as the use of gel electrophoresis coupled to mass spectroscopy to identify multiple proteins in a sample.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include, but are not limited to, those described in U.S. Pat. Nos. 5,885,530; 4,981,785; 6,159,750; and 5,358,691, each of which is herein incorporated by reference. In some embodiments, the analysis and presentation of results is also automated. For example, in some embodiments, software that generates a diagnosis and/or prognosis based on the presence or absence of a series of proteins corresponding to markers is utilized.

In other embodiments, the immunoassay described in U.S. Pat. Nos. 5,599,677 and 5,672,480, each of which is herein incorporated by reference, is utilized. In other embodiments, proteins are detected by immunohistochemistry. In still other embodiments, markers are detected at the level of cDNA or RNA.

As used herein, the term “gene expression biomarkers” shall mean any biologic marker which can indicate the rate or degree of gene expression of a specific gene including, but not limited to, mRNA, cDNA or the polypeptide expression product of the specific gene.

In some embodiments of the present invention, gene expression biomarkers are detected using a PCR-based assay. In yet other embodiments, reverse-transcriptase PCR (RT-PCR) is used to detect the expression of RNA. In RT-PCR, RNA is enzymatically converted to cDNA using a reverse-transcriptase enzyme. The cDNA is then used as a template for a PCR reaction. PCR products can be detected by any suitable method including, but not limited to, gel electrophoresis and staining with a DNA-specific stain or hybridization to a labeled probe.

In some embodiments, the quantitative RT-PCR with standardized mixtures of competitive templates method described in U.S. Pat. Nos. 5,639,606; 5,643, 765; and 5,876,978, each of which is herein incorporated by reference, is utilized.

In preferred embodiments of the present invention, gene expression biomarkers are detected using a hybridization assay. In a hybridization assay, the presence or absence of a marker is determined based on the ability of the nucleic acid from the sample to hybridize to a complementary nucleic acid molecule, e.g., an oligonucleotide probe. A variety of hybridization assays are available.

In some embodiments, hybridization of a probe to the sequence of interest is detected directly by visualizing a bound probe, e.g., a Northern or Southern assay. See, e.g., Ausabel et al., eds., Current Protocols in Molecular Biology, John Wiley & Sons, NY (1991). In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide, is allowed to contact the membrane under low-, medium- or high-stringency conditions. Unbound probe is removed and the presence of binding is detected by visualizing the labeled probe.

In some embodiments, the DNA chip assay is a GeneChip (Affymetrix, Santa Clara, Calif.). See, e.g., U.S. Pat. Nos. 6,045,996; 5,925,525; and 5,858,659, each of which is herein incorporated by reference. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementary, the identity of the target nucleic acid applied to the probe array can be determined.

In other embodiments, a DNA microchip containing electronically captured probes (Nanogen, San Diego, Calif.) is utilized. See, e.g., U.S. Pat. Nos. 6,017,696; 6,068,818; and 6,051,380, each of which are herein incorporated by reference. Through the use of microelectronics, Nanogen's technology enables the active movement and concentration of charged molecules to and from designated test sites on its semiconductor microchip. DNA capture probes unique to a given gene expression biomarkers are electronically placed at, or “addressed” to, specific sites on the microchip. Since nucleic acid molecules have a strong negative charge, they can be electronically moved to an area of positive charge.

In still further embodiments, an array technology based upon the segregation of fluids on a flat surface (chip) by differences in surface tension (ProtoGene, Palo Alto, Calif.) is utilized. See, e.g., U.S. Pat. Nos. 6,001,311; 5,985,551; and 5,474,796, each of which is herein incorporated by reference. Protogene's technology is based on the fact that fluids can be segregated on a flat surface by differences in surface tension that have been imparted by chemical coatings. Once so segregated, oligonucleotide probes are synthesized directly on the chip by ink-jet printing of reagents.

In yet other embodiments, a “bead array” is used for the detection of gene expression biomarkers (Illumina, San Diego, Calif.). See, e.g., PCT Publications WO 99/67641 and WO 00/39587, each of which is herein incorporated by reference. Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given marker. Batches of beads are combined to form a pool specific to the array. To perform an assay, the BEAD ARRAY is contacted with a prepared sample. Hybridization is detected using any suitable method.

In some preferred embodiments of the present invention, hybridization is detected by enzymatic cleavage of specific structures, e.g., INVADER™ assay, Third Wave Technologies. See, e.g., U.S. Pat. Nos. 5,846,717, 6,090, 543; 6,001,567; 5,985,557; and 5,994,069, each of which is herein incorporated by reference. In some embodiments, hybridization of a bound probe is detected using a TaqMan assay (PE Biosystems, Foster City, Calif.). See, e.g., U.S. Pat. Nos. 5,962,233 and 5,538,848, each of which is herein incorporated by reference. The assay is performed during a PCR reaction. The TaqMan assay exploits the 5′-3′ exonuclease activity of DNA polymerases, such as AMPLITAQ DNA polymerase. A probe, specific for a given marker, is included in the PCR reaction. The probe consists of an oligonucleotide with a 5′-reporter dye, e.g., a fluorescent dye and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

Additional detection assays that are produced and utilized using the systems and methods of the present invention include, but are not limited to, enzyme mismatch cleavage methods, e.g., Variagenics (see U.S. Pat. Nos. 6,110,684; 5,958,692; and 5,851,770, herein incorporated by reference in their entireties); branched hybridization methods, e.g., Chiron (see U.S. Pat. Nos. 5,849,481; 5,710,264; 5,124,246; and 5,624,802, herein incorporated by reference in their entireties); rolling circle replication (see, e.g., U.S. Pat. Nos. 6,210,884 and 6,183,960, herein incorporated by reference in their entireties); NASBA (see, e.g., U.S. Pat. No. 5,409,818, herein incorporated by reference in its entirety); molecular beacon technology (see, e.g., U.S. Pat. No. 6,150,097, herein incorporated by reference in its entirety); E-sensor technology (see Motorola, U.S. Pat. Nos. 6,248,229; 6,221,583; 6,013,170; and 6,063,573, herein incorporated by reference in their entireties); cycling probe technology (see, e.g., U.S. Pat. Nos. 5,403,711; 5,011,769; and 5,660,988, herein incorporated by reference in their entireties); ligase chain reaction [see Barnay, Proc. Natl. Acad. Sci. USA, Vol. 88, pp. 189-93 (1991)]; and sandwich hybridization methods (see, e.g., U.S. Pat. No. 5,288,609, herein incorporated by reference in its entirety).

In some embodiments, mass spectroscopy is used to detect gene expression biomarkers. For example, in some embodiments, a MASSARRAY™ system (Sequenom, San Diego, Calif.) is used to detect gene expression biomarkers. See, e.g., U.S. Pat. Nos. 6,043,031; 5,777,324; and 5,605,798, each of which is herein incorporated by reference.

In some embodiments, the present invention provides kits for the identification, characterization and quantitation of gene expression biomarkers. In some embodiments, the kits contain antibodies specific for gene expression biomarkers, in addition to detection reagents and buffers. In other embodiments, the kits contain reagents specific for the detection of nucleic acid, e.g., oligonucleotide probes or primers. In preferred embodiments, the kits contain all of the components necessary to perform a detection assay, including all controls, directions for performing assays and any necessary software for analysis and presentation of results. In some embodiments, the kits contain instructions including a statement of intended use as required by the Environmental Protection Agency or U.S. Food and Drug Administration (FDA) for the labeling of in vitro diagnostic assays and/or of pharmaceutical or food products.

Comparison of the organism's gene expression pattern, with the result expressed in Table 9, would indicate whether the organism has a gene expression profile which may indicate that the organism does or does not contain ovarian cancer.

In another embodiment, the present invention is a method of screening a test compound for the ability to inhibit, retard, reverse or mimic the gene expression changes characteristic of ovarian cancer. In a typical example of this embodiment, one would first treat a test mammal known to have ovarian cancer with a test compound and then analyze a representative tissue of the mammal for the level of expression of the genes or sequences which change in expression in response to ovarian cancer. Preferably, the tissue is biopsy material from the tumor or, in a preferred embodiment, an easily obtainable tissue, such as blood or serum.

One then compares the analysis of the tissue with a control mammal known to have ovarian cancer but not given the test compound and thereby identifies test compounds that are capable of modifying the expression of the gene expression biomarkers sequences in the mammalian samples such that the expression is altered toward the No Ovarian Cancer pattern.

In another embodiment of the present invention, one would use the sequences of the genes disclosed in Table 2 for a therapy for mimicking the No Ovarian Cancer state. In general, one would try to amplify gene expression for the genes identified herein as under-expressed in ovarian cancer and decrease the expression of genes identified herein as over-expressed in ovarian cancer. For example, one might try to decrease the expression of genes or sequences identified in Table 2 as increased or increase the expression of genes found to be decreased in ovarian cancer.

Methods of increasing and decreasing expression would be known to one of skill in the art. Examples for supplementation of expression would include supplying the organism with additional copies of the gene. A preferred example for decreasing expression would include RNA antisense technologies or pharmaceutical intervention. The genes disclosed in Table 2 would be appropriate drug development targets. One would use the information presented in the present application for drug development by using currently existing, or by developing, pharmaceutical compounds that either mimic or inhibit the activity of the genes listed in Table 2, or the proteins encoded by these genes. Therefore, the gene expression biomarkers or genes disclosed herein represent targets for pharmaceutical development and gene therapy or RNA antisense therapy with the goal of suppressing the changes characteristic of ovarian cancer at the molecular level. These gene expression alterations may also play a role in understanding the various mechanisms that underlie ovarian cancer. Additionally, these genes represent biomarkers of ovarian cancer that can be used for diagnostic purposes.

The present invention is not limited by the form of the expression profile. In some embodiments, the expression profile is maintained in computer software. In some embodiments, the expression profile is written material. The present invention is not limited by the number of markers provided or displayed in an expression profile. For example, the expression profile may comprise two or more markers found in Table 2, indicating a biological status of a sample.

The present invention further provides databases comprising expression information, e.g., expression profiles comprising one or more markers from Table 2 from one or more samples. In some embodiments, the databases find use in data analysis including, but not limited to, comparison of markers to one or more public or private information databases, e.g., OMIM, GenBank, BLAST, Molecular Modeling Databases, Medline, genome databases, etc. In some such embodiments, an automated process is carried out to automatically associate information obtained from data obtained using the methods of the present invention to information in one or more of public or private databases. Associations find use, e.g., in making expression correlations to phenotypes, e.g., disease states.

We also understand the present invention to be extended to mammalian homologues of the mouse genes listed in Table 9. One of skill in the art could easily investigate homologues in other mammalian species by identifying particular genes with sufficiently high homology to the genes listed in Table 9. By “high homology” we mean that the homology is at least 50% overall (within the entire gene or protein) either at the nucleotide or amino acid level.

### List of Abbreviations

A Absent
AvgDiff Average Difference (overall intensity of probe set on Affymetrix array)
CHTN Cooperative Human Tissue Network
CI Confidence Interval
FIGO Federation of Gynecology and Obstetrics
GAPDH Glyceraldehyde 3-phosphate dehydrogenase
GNF Genomics Institute of the Novartis Research Foundation
mg Milligram
NCBI National Center for Biotechnology Information
Neg Negative
nM Nanometer
OMIM Online Mendelian Inheritance in Man
OR Odds Ratio
ORF Open Reading Frame
P Present
PG Pharmacogenetics
Pos Positive
QC Quality Control
RNA Ribonucleic Acid

### EXAMPLE 1

Preferred Methods

To identify genes involved in the development and progression of ovarian tumors, we compared the gene expression profiles of a series of Normal and Tumor ovarian biopsies. Gene expression data for more than 12,000 genes were generated from each sample. Of the 900 probe sets that we observed to be most differentially-expressed between the Normal and cancerous ovarian biopsies, 98% were down-regulated in the Tumor biopsies. Using 8 Normal and 10 Tumor samples, we identified a minimum number of probe sets (28) that could be used to classify biopsies as Normal or Tumor. This finding was validated on a second set of biopsies (4 Normal and 14 Tumor) previously profiled by another laboratory. A mean Normal ovarian profile was established that could be used as a reference to compare other ovarian biopsies. The identification of the most differentially-expressed genes between Normal and Tumor ovarian biopsies may provide new insight into the molecular mechanisms of ovarian tumor development and progression. Some of the genes identified in this study are known to be involved in ovarian cancer, but a large proportion represents novel candidates for drug targets and molecular biomarkers to diagnose or monitor disease and treatment.

Materials and Methods

Samples

Flash-frozen ovarian biopsies were obtained from Asterand (Detroit, Mich.), and consisted of 10 Tumor samples and 10 adjacent Normal tissues. Total RNA was also purchased for 4 additional samples from Ambion (Austin, Tex.) and Stratagene (La Jolla, Calif.). Gene expression profiles from samples used in the validation step had been previously generated at GNF and reported. See Welsh et al. (2001), supra.

Most of the tumors analyzed were malignant surface epithelial serous tumors, e.g., papillary cystcarcinoma, papillary cystadenocarcinoma or papillary cystcarcinoma; others included a mucinous cyst carcinoma, an endometrioid adenocarcinoma and a mature teratoma.

A summary of sample information is shown in Table 1 below.

TABLE 1
Ovarian Samples Used for Gene Expression Analysis

| Sample | | | Tumor | | |
|---|---|---|---|---|---|
| code | Status | Comment/Tumor Type | Cell (%) | Stage | Source |
| p2437 | Normal | Normal margin to cystadenoma | | | Stratagene |
| p2709 | Normal | No pathology data | | | Ambion |
| p5720 | Normal | | | | Ambion |
| p5721 | Tumor | Adenocarcinoma | | Not | Ambion |
| | | | | Available | |
| p6166 | Tumor | Serous cyst carcinoma | 70 | III | Asterand |
| p6167 | Normal | | | | Asterand |
| p6168 | Tumor | Serous cyst carcinoma | 50 | II | Asterand |
| p6169 | Normal | | | | Asterand |
| p6170 | Tumor | Serous cyst carcinoma | 80 | IC | Asterand |
| p6171 | Normal | | | | Asterand |
| p6172 | Tumor | Endometrioid adenocarcinoma | 50 | IV | Asterand |
| p6173 | Normal | | | | Asterand |
| p6174 | Tumor | Mucinous cyst carcinoma | 40 | I | Asterand |
| p6175 | Normal | | | | Asterand |
| p6176 | Tumor | Papillary serous cyst carcinoma | 80 | III | Asterand |
| p6177 | Normal | | | | Asterand |
| p6178 | Tumor | Serous cyst carcinoma | 70 | Not | Asterand |
| | | | | Available | |
| p6179 | Normal | | | | Asterand |
| p6180 | Tumor | Serous cyst carcinoma | 70 | Not | Asterand |
| | | | | Available | |
| p6181 | Normal | | | | Asterand |
| p6182 | Tumor | Serous cyst carcinoma | 70 | IIIC | Asterand |
| p6183 | Normal | | | | Asterand |
| p6184 | Tumor | Mature teratoma | 70 | III | Asterand |
| p6185 | Normal | | | | Asterand |
| OVR1T | Tumor | Adenocarcinoma, serous papillary | 40 | IIIC | CHTN |
| OVR2T | Tumor | Adenocarcinoma, serous papillary | 60 | IIIC | CHTN |
| OVR5T | Tumor | Adenocarcinoma, serous papillary | 80 | IIIB | CHTN |
| OVR8T | Tumor | Adenocarcinoma, serous papillary | 80 | IVA | CHTN |
| OVR10T | Tumor | Adenocarcinoma, serous papillary | 40 | IVA | CHTN |
| OVR11T | Tumor | Adenocarcinoma, serous papillary | 40 | IIIC | CHTN |
| OVR12T | Tumor | Adenocarcinoma, serous papillary | 50 | IIIC | CHTN |
| OVR13T | Tumor | Adenocarcinoma, serous papillary | 90 | IIIC | CHTN |
| OVR16T | Tumor | Adenocarcinoma, serous papillary | 40 | IIIC | CHTN |
| OVR19T | Tumor | Adenocarcinoma, serous papillary | 30 | IIIC | CHTN |
| OVR22T | Tumor | Adenocarcinoma, serous papillary | 40 | IIIC | CHTN |
| OVR26T | Tumor | Adenocarcinoma, serous papillary | 60 | IIIC | CHTN |
| OVR27T | Tumor | Adenocarcinoma, serous papillary | 40 | IIIC | CHTN |
| OVR28T | Tumor | Adenocarcinoma, serous papillary | 80 | IIIC | CHTN |
| OVR102N | Normal | | | | BioChain |
| | | | | | Institute |
| OVR278EN | Normal | Enriched for epithelium | | | BioChain |
| | | | | | Institute |
| OVR278SN | Normal | Enriched for stroma | | | BioChain |
| | | | | | Institute |
| HUOVR | Normal | | | | BioChain |
| | | | | | Institute |

Note:
Paired samples (Normal and Tumor adjacent tissue) obtained from the same patient are boxed together. Stages of ovarian cancers are indicated using the FIGO staging system.

RNA Expression Profiling

Total RNA was extracted from each biopsy and processed as previously described. RNA extraction techniques are well-known to those of skill in the art. All samples profiled were processed using the Affymetrix GENECHIP™ system as recommended by Affymetrix (GeneChip Expression Analysis Technical Manual, rev. 1, July 2001). Concentration and total amount of RNA and cRNA were estimated by measuring the samples at 260 nM and 280 nM wavelengths using a Beckman-Coulter DU 650 spectrophotometer after a 1:50 dilution of the samples (see Table 2). The type of array used for this study was the Human Genome U95Av2 (http://www.affymetrix.com/products/arrays/specific/hgu95.affx).

Analytical Strategy

Analysis of the expression profiles was performed in several steps described below.

Selection of Microarray Data of Highest Quality

We used for our analysis only microarrays for which the scaling factor was lower than 6, and where more than 30% of the probe sets were called “Present” by the Affymetrix MAS 4.0 algorithm.

Selection of a Subset of Probe Sets

Expression data were directly imported into the GENE SPRING® program (Silicon Genetics, Redwood City, Calif.) from the database. Genes expressed in only a few samples were eliminated; out of the 12,627 probe sets on the microarray, only those with an AvgDiff of at least 100 in 10% of the samples or more were used for further analysis. A clustering experiment was performed to visualize the different gene expression profiles of Normal and Tumor biopsies.

Further filtering was accomplished by eliminating probe sets of low quality or very low intensity signals in both groups of samples (Group 1: Normal biopsies; Group 2: Tumor biopsies). Probe sets not called “Present” (P) in at least 75% of the samples in one of the two groups were not used for further analysis. In addition, AvgDiff values lower than 20 were all converted to a value of 20.

Focus on the Most Differentially-Expressed Genes

Selection of genes differentially-expressed between the two groups of samples was done in 2 steps:
1. The AvgDiff of each probe set was compared between the 2 groups of samples by a non-parametric one-way ANOVA test, using SAS 8.2.
2. The AvgDiff of each probe sets with p<0.05 was then correlated with the group of samples (Normal or Tumor). Probe sets were ranked from highest absolute PCC to lowest (calculated in Microsoft Excel).
   Re-Classification of Samples

We used the “leave-one-out” analytical strategy previously described to determine the optimal number of probe sets that distinguished an ovarian tumor from a normal ovarian tissue. See van't Veer et al. (2002), supra.

For every sample left-out, we determined the average AvgDiff of each probe set in each group of samples (Groups 1 and 2). PCCs between the expression profile of the left-out sample and the average profile of each group were calculated for each probe set. The effectiveness of each probe set in distinguishing a tumor from a normal ovarian tissue was evaluated by re-classifying each sample as Normal or Tumor based on the higher of the two CCs.

We determined the number of misclassified samples when using increasingly larger sets of genes (starting with 5). As used herein, a “false Neg” is defined as a Tumor incorrectly classified as a Normal ovary tissue, and inversely, a “false Pos” is defined as a Normal tissue incorrectly classified as a Tumor.

The probe sets that most-effectively distinguished tumor from normal ovarian tissue were then tested in their ability to classify gene expression profiles of a different set of ovarian tissues (Normal and Tumor) generated at GNF (see Table 1).

Statistical Determination of OR

Using the desired threshold correlation value, a 2×2 table was constructed indicating the number of biopsies correctly and incorrectly identified as Normal or Tumor. ORs, along with 95% Cis, were calculated using SAS version 8.2. Statistical significance was determined using a Fisher's exact test with p-value cut-off of 0.05.

Genes

The link between a probe set name and a GenBank Accession Number was provided by Affymetrix, together with a short gene description. We complemented and updated this description by a search of the NCBI databases, mainly LocusLink (http://www.ncbi.nlm.nih.gov/LocusLink/index.html), OMIM (http://www.ncbi.nlm.nih.gov/Omim/searchomim.html) and PubMed (http://www.ncbi.nlm.nih.gov/entrez/query.fcgi).

Results

RNA Expression Profiling

Eighteen out of the 20 biopsies yielded more than the 5 mg of purified total RNA necessary to process the samples further (see Table 2). Sample p6175, from which less than 1 mg of purified total RNA was obtained, was the smallest sample (38 mg). The quality of the RNA was assessed by electrophoresis on a 1% agarose gel. The absence of both 28 S and 18 S ribosomal RNA bands was observed for samples p6169 and p6180, indicating some RNA degradation. For microarray hybridization, a maximum of 15 mg of cRNA was used when available, but no less than 12 mg. Enough cRNA was available for 21 samples (see Table 2).

Quality Assessment

The data from the 21 arrays hybridized in PG (this study) and from the 18 hybridized previously at GNF were checked for quality (see Table 3). See Welsh et al. (2001), supra. All but 3 (p6169, p6180 and p6185) passed our criteria of a scaling factor lower than 6, with more than 30% of probe sets called “P” (see Table 3). The 36 remaining expression profiles were separated into 2 sets: a test set of 18 profiles generated in PG consisting of data from 8 Normal and 10 Tumor biopsies, and a validation set of 18 profiles previously generated at GNF from 4 Normal and 14 Tumor biopsies. See Welsh et al. (2001), supra.

Analysis

Clustering Analysis

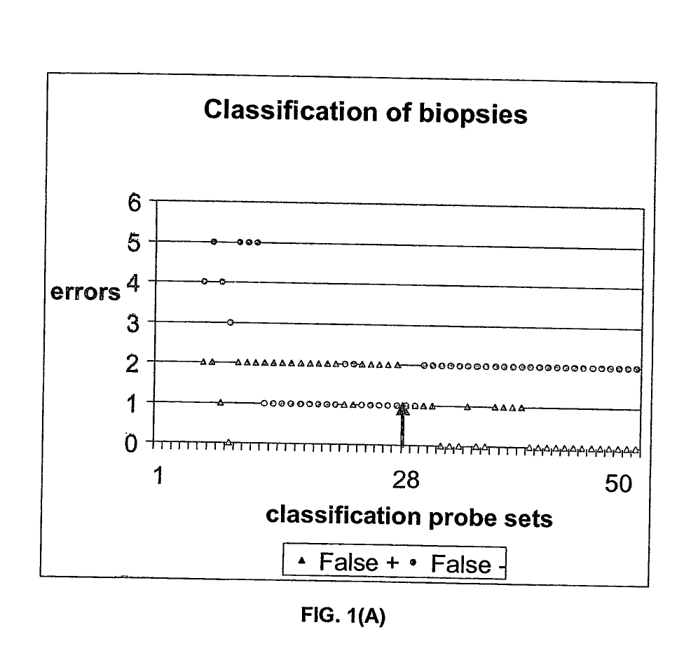

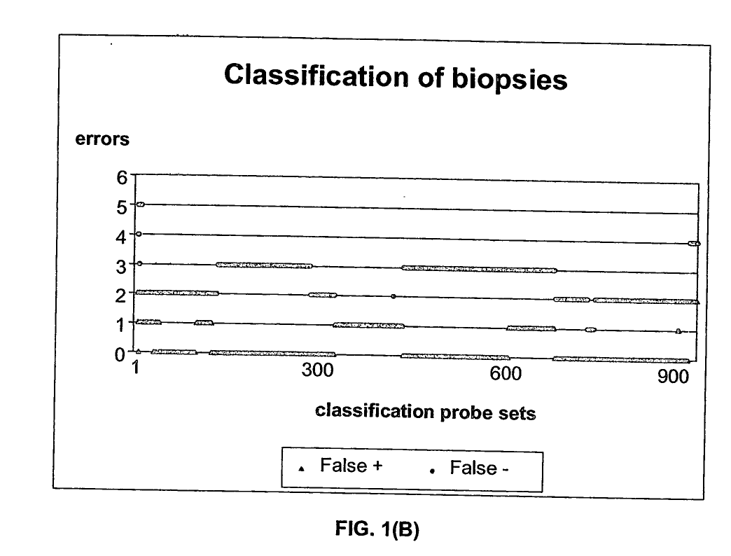

Expression data of the 18 samples of the test set were imported into the GENESPRING® software. Out of the 12,627 probe sets on the Affymetrix U95A microarray, 2,174 had an AvgDiff of at least 100 in 2 or more of these 18 samples and were used for clustering analysis. The resulting clustering tree of samples and probe sets is shown in FIG. 1. Interestingly, the dendogram of experiments contains two main branches corresponding to the two groups of samples, Normal (top) and Tumor (bottom) biopsies. The vast majority (>90%) of genes examined have an overall higher expression in the Normal ovarian tissues. The dendogram of probe sets shows only a small cluster of genes with higher expression in the Tumor tissues (left part).

Selection of the Most Differentially-Expressed Genes

Out of the 2,174 probe sets, 217 were excluded from further analysis because they provided large number of “A” or “marginal” calls (>75% in both groups).

Data for the remaining 1,957 probe sets were exported into SAS version 8.2 for non-parametric one-way ANOVA testing between the Normal and the Tumor groups. A total of 900 probe sets had AvgDiff values significantly different between the two groups (p<0.05). These genes are listed in Table 9.

The AvgDiff of these 900 probe sets was then correlated with the two groups of samples (Group 1: Normal; Group 2: Tumor). The absolute PCC (R)-values ranged from 0.042-0.877, with 694 probe sets (77%) with a R-value higher than 0.5. The AvgDiff data of the 900 probe sets ranked from highest absolute PCC to lowest are available in Appendix 1.

Leave-One-Out Method and Re-Classification of Samples

The “leave-one-out” analytical strategy previously described was applied to the 18 ovarian samples for the expression of the 900 selected probe sets. See van't Veer et al. (2002), supra.

The number of misclassified samples when using the first 5 probe sets was 6 (2 false Pos and 4 false Neg). Increasingly large sets of genes were used. The number of misclassifications varied between 2 and 7, with the minimum achieved when using the first 28 probe sets (FIG. 1). These first 28 probe sets displayed only one false Pos and one false Neg. Interestingly, perfect classification of Normal biopsies (0 false Pos) was achieved with the first 32 probe sets (which also detected 2 false Neg), while perfect classification of Tumor biopsies (0 false Neg) was never seen.

Optimal Classification Set and Correlation Threshold Values

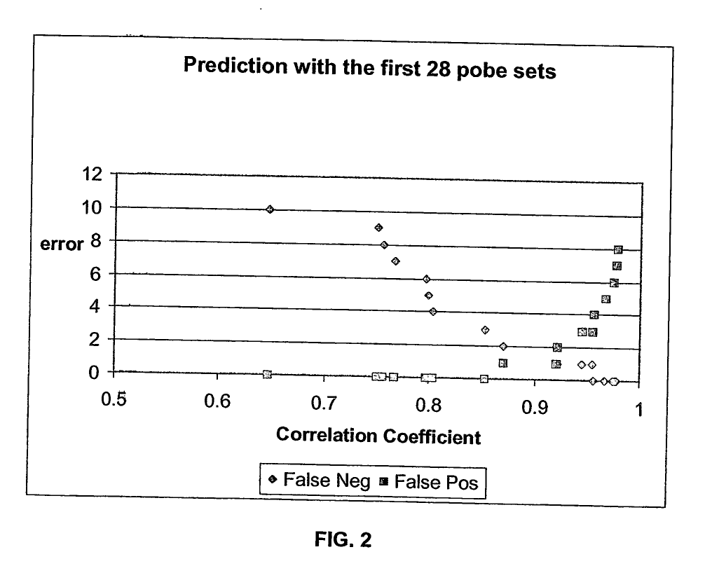

We determined the mean Normal (No Tumor) biopsy profile for the classification probe sets, to be used as a reference for analysis of biopsies of unknown or questionable status; we expected that tumor heterogeneity may not allow the determination of a reference Tumor profile. We examined the classification value of the first 28 probe sets, by comparing their expression for each of the 18 samples to the mean Normal profile calculated using all 8 Normal biopsy profiles. Samples were then ranked by correlation values from highest to lowest and error rates were determined as a function of where the threshold correlation was drawn. The results are displayed in FIG. 2. The minimum number of incorrectly assigned samples was 2 [1 false Pos (p6177) and 1 false Neg (p6168)]. The corresponding CC value was between 0.920 and 0.921. The OR and Fisher's exact test were performed based on the number of samples correctly and incorrectly predicted to be Normal or Tumor. The difference between the observed and expected biopsy status was significant: OR=63; 95% CI: 3.3-1194.7, p=0.0029. The OR indicates that an ovarian biopsy of the test set is nearly 63 times more likely to be from an ovarian tumor if its expression profile of the 28 predictor probe sets correlates with the mean Normal profile with a CC≦0.920 (see Table 4). In our test set, 100% of profiles with a CC>0.955 correspond to Normal biopsies and 100% of profiles with a CC<0.870 correspond to Tumor biopsies (see FIG. 3).

Validation of The Mean Normal Profile

The 28 probe sets selected by the leave-one-out method allowed us to distinguish Normal from Tumor ovarian biopsies in our series of 18 ovarian samples. We then tested if independent ovarian biopsies could be correctly classified by comparing their expression profile to the same mean Normal profile of the 28 classification probe sets.

FIG. 3 summarizes the classification of all ovarian biopsies based on the correlation of 28 probe sets. Remarkably, the profiles of the Normal and Tumor samples of the validation set were clearly separated from each other (see FIG. 3). As in the test set, 100% of profiles with a CC<0.870 correspond to Tumor biopsies. Interestingly, Normal profiles had lower CCs than in the test set, and the threshold correlation value that best separate Normal and Tumor biopsies in the validation set lies between 0.762 and 0.876.

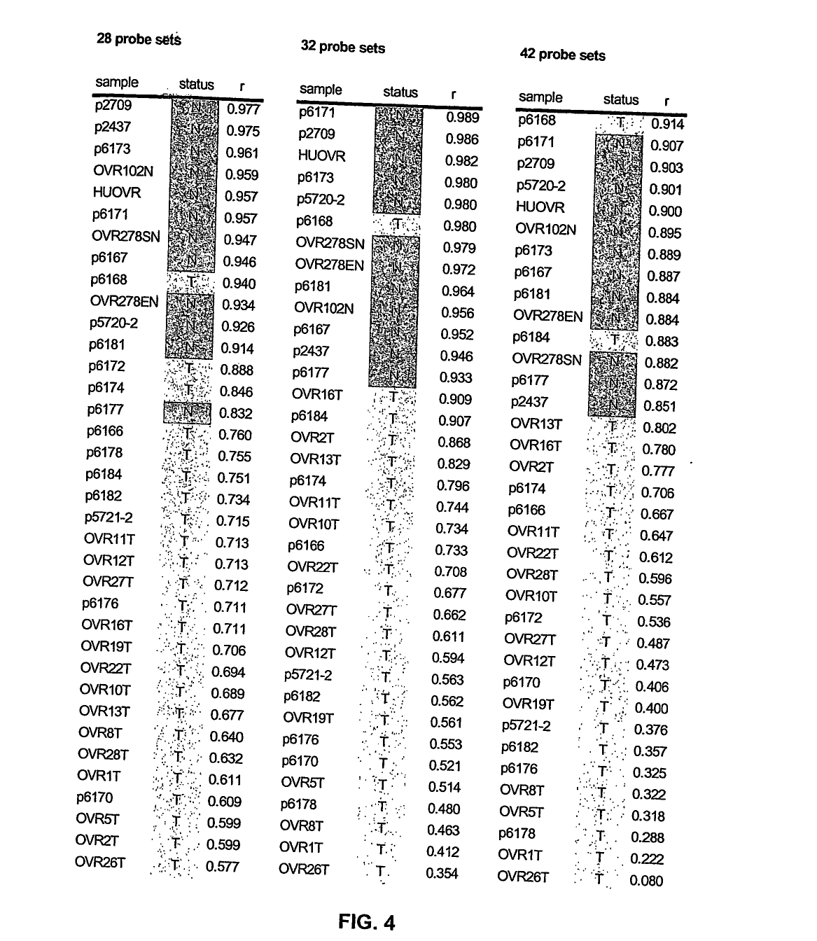

We performed a non-parametric t-test between the average Normal profile of the test set and the average Normal profile of the validation set. Similarly, we compared the average Tumor profiles of both sets. Since no statistical difference was observed (p=0.373 and p=0.110, respectively), we combined both sets to increase the classification value of the 28 probe sets. We compared the expression for all the samples to the mean Normal profile calculated using all 12 Normal biopsy profiles (8 from the test set and 4 from the validation test). Results confirm that correlation values provide highly-significant separation of the Normal biopsy from the Tumor biopsy profiles (see FIG. 4 and Table 5). As seen previously, the profile of Tumor sample (p6168) had a high correlation with the average Normal profile.

Correlation Between Individual Gene Expression and Biopsy Status

The selection of probe sets for the classification of ovarian biopsies was originally done based on the profile of the 18 test samples. The good separation of all 36 Normal and Tumor samples (see FIG. 4) with the same probe sets suggested that the genes selected by our method are differentially-expressed in many other ovarian tumors. However, because of tumor heterogeneity, the difference in individual gene expression is likely to vary with the samples analyzed. We evaluated to what extent the 900 probe sets differentially-expressed in the 18 test samples, were also differentially-expressed when all 36 biopsies were analyzed.

Probe sets were ranked from highest absolute PCC to lowest, first using the 18 samples from the test set, and then with all 36 samples from both the test set and the validation set. From the 900 probe sets selected, 694 and 473 had an absolute CC higher than 0.5 with the 18 and 36 samples, respectively; 412 probe sets had a coefficient higher than 0.5 in both cases. Interestingly, from the 28 probe sets originally selected for the biopsy classification, 19 ranked in the top 100; the other 9 probe sets had correlation values ranging from 0.359-0.703.

Genes Differentially-Expressed Between Normal and Tumor Ovarian Biopsies Genes Up-Regulated in Ovarian Tumors

Among the genes differentially expressed between Normal and Tumor ovarian biopsies, we detected a few genes already known to be up-regulated in ovarian tumors, such as the genes coding for Claudin 4, topoisomerase II alpha, Kallikrein 8, osteopontin, as well as potential new markers of ovarian cancers (see Table 6).

Claudin 4, a component of tight junctions, has been shown to be up-regulated in ovarian tumors together with another member of this family of transmembrane receptors, Claudin 3. See Hough et al., Cancer Res., Vol. 60, No. 22, pp. 6281-6287 (2000). Costa and colleagues have reported that levels of topoisomerase II alpha correlate with poor prognosis of ovarian surface epithelial neoplasms. Kallikrein 8 has been detected by immunohistochemistry in carcinoma but not Normal ovarian tissue and was suggested as a prognostic marker of ovarian cancer. See Underwood et al., Cancer Res., Vol. 59, No. 17, pp. 4435-4439 (1999); and Magkiara et al., Clin. Cancer Res., Vol. 7, No. 4, pp. 806-811 (2001). Osteopontin has also been previously proposed as a diagnostic biomarker for ovarian cancer. See Kim et al., JAMA, Vol. 287, No. 13, pp. 1671-1679 (2002). Another gene, C20ORF1, has been shown to be expressed in lung carcinoma cell lines but not in normal lung tissues. See Manda et al., Genomics, Vol. 61, No. 1, pp. 5-14 (1999). Other genes that may have be over-expressed in only some of the biopsies due to the tumor type, the disease stage or other tumor specificity, were not detected by our analytical method.

Genes Down-Regulated in Ovarian Tumors

We further examined a large number of genes down-regulated in the ovarian tumor biopsies profiled. For analysis purpose, we classified the 28 probe sets and the top 100 down-regulated genes in 8 categories based on the known or suspected function of their product (see Tables 7 and 8). Interestingly, the function of nearly 30% of these 100 genes is still unknown. Most of the other genes play a role in, or are already suspected to be involved in transcription regulation (16 genes), in cell cycle regulation, growth differentiation, cell death or tumor suppression (12 genes) and signal transduction (6 genes). This list includes several potential tumor suppressors: the gene coding for the transforming growth factor beta receptor III (TGFβR3), a platelet-derived growth factor receptor-like gene (PDGFRL), the suppression of tumorigenicity (ST13) gene, a gene coding for a reversion-inducing-cysteine-rich protein with kazal motif (RECK) and the paternally expressed 3 (PEG3) gene.

This observation suggests that the genes with still unidentified function are likely to be involved in cell cycle regulation, growth differentiation, signal transduction or transcription regulation. Some of them may act as tumor suppressors. Down-regulation in ovarian tumor or cell lines had been reported for just one of these genes, IGFBP5 which in our study was detected with 2 separate probe sets (see Table 8). See Welsh et al. (2001), supra.

Only 6 genes coding for proteins of the extracellular matrix were noticed including laminin alpha 2 (LAMα2). Yang and colleagues have reported that transient loss of LAMα2 in the basement membrane of the pre-malignant epithelium and subsequent inactivation of Dab2 are common early event associated with tumorigenicity of the ovarian surface epithelium. See Yang et al., Cancer, Vol. 94, No. 9, pp. 2380-2392 (2002). Interestingly, down-regulation of Dab2 (probe set 479_at) was also observed in our study with a CC value of 0.49.

Taken together, these results indicated that most of the genes with a statistically significant decreased expression in the ovarian biopsies, are indeed involved in the development or progression of the tumors rather than detected because of a change in cell population or tissue organization, e.g., loss of connective tissue and fat cells.

Discussion

The filtering and analytical methods that we used here, provided a list of genes differentially expressed between Normal and Tumor ovarian samples. We showed that a small subset (28-42 probe sets) is sufficient to accurately classify ovarian biopsies as Normal or Tumor based on their expression profiles. Validation of this expression signature was done on different biopsies profiled in an independent laboratory, and confirms that the difference in expression observed between the Normal or Tumor samples reflects a biological process rather than of a laboratory or analytical error.

Several factors not examined here that may affect the detection of differentially expressed genes include the number of samples in the test set, and the heterogeneity of the samples studied. Indeed, it is expected that biopsies and, in particular, Tumor biopsies, have a substantial level of heterogeneity: tumor type, grade, percentage of tumor cells, presence of connective and fat tissues, etc. We studied different types of ovarian tumors of various grades (see Table 1) to search for genes involved in common pathways of tumor development and progression, rather than genes involved more specifically in certain types of tumors as previously reported. See Ono et al. (2000), supra; and Welsh et al. (2001), supra.

Our clustering analysis of the biopsy expression profiles, revealed that the vast majority of genes that differentiate Nonnal and Tumor samples were down-regulated in the tumors. Indeed, when the 900 most differentially-expressed probes were ranked based on the CC between their expression in all 36 biopsies and the Normal and Tumor status, the top 220 probes (R from 0.865-0.644) were down-regulated in the tumors. We examined more closely the function of the top 100 genes (R from 0.865-0.72), and the top 10 genes over-expressed in the tumors (R from 0.643-0.443). Among the most differentially-expressed genes, we detected several genes already known to be up-regulated in ovarian tumors, as well as potential new markers of ovarian cancers. However, most of the genes were down-regulated, most likely because we studied various types of late stage tumors of different origins, different grades and different tumor cell content. The involvement of many of these genes in transcription regulation, in cell cycle regulation, growth differentiation, signal transduction, cell death or tumor suppression underscores the need to further evaluate their role in ovarian cancer. The list of other genes of still unknown function points to novel potential players in tumor development and progression.

Methods of Modifying RNA Abundances or Activities

Methods of modifying RNA abundances and activities currently fall within three classes: ribozymes, antisense species and RNA aptamers. See Good et al., Gene Ther., Vol. 4, No.1, pp. 45-54 (1997). Controllable application or exposure of a cell to these entities permits controllable perturbation of RNA abundances.

Ribozymes

Ribozymes are RNAs which are capable of catalyzing RNA cleavage reactions. See Cech, Science, Vol. 236, pp. 1532-1539 (1987); PCT International Publication WO 90/11364 (1990); Sarver et al., Science, Vol. 247, pp. 1222-1225 (1990). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. See Haseloff et al., Nature, Vol. 334, pp. 585-591 (1988); Koizumi et al., FEBS Lett., Vol. 228, pp. 228-230 (1988); and Koizumi et al., FEBS Lett., Vol. 239, pp. 285-288 (1988). Ribozyme methods involve exposing a cell to, inducing expression in a cell, etc. of such small RNA ribozyme molecules. See Grassi and Marini, Annals of Med., Vol. 28, No. 6, pp. 499-510 (1996); and Gibson, Cancer Meta. Rev., Vol. 15, pp. 287-299 (1996).

Ribozymes can be routinely expressed in vivo in sufficient number to be catalytically effective in cleaving mRNA, and thereby modifying mRNA abundances in a cell. See Cotton et al., EMBO J., Vol. 8, pp. 3861-3866 (1989). In particular, a ribozyme coding DNA sequence, designed according to the previous rules and synthesized, e.g., by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter, e.g., a glucocorticoid or a tetracycline esponse element, is also introduced into this construct so that ribozyme expression can be selectively controlled. For saturating use, a highly and constituently active promoter can be used. tDNA genes, i.e., genes encoding tRNAs, are useful in this application because of their small size, high rate of transcription and ubiquitous expression in different kinds of tissues. Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly, the abundance of virtually any RNA species in a cell can be modified or perturbed.

Antisense Molecules

In another embodiment, activity of a target RNA (preferably mRNA) species, specifically its rate of translation, can be controllably inhibited by the controllable application of antisense nucleic acids. Application at high levels results in a saturating inhibition. An “antisense” nucleic acid as used herein refers to a nucleic acid capable of hybridizing to a sequence-specific, e.g., non-poly A, portion of the target RNA, e.g., its translation initiation region, by virtue of some sequence complementary to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides, ranging from 6 oligonucleotides to about 200 oligonucleotides. In specific aspects, the oligonucleotide is at least 10 nucleotides, at least 15 nucleotides, at least 100 nucleotides or at least 200 nucleotides. The oligonucleotides can be DNA or RNA or chimeric mixtures or derivatives or modified versions thereof, single-stranded or double-stranded. The oligonucleotide can be modified at the base moiety, sugar moiety or phosphate backbone. The oligonucleotide may include other appending groups, such as peptides, or agents facilitating transport across the cell membrane [see, e.g., Letsinger et al., Proc. Natl. Acad. Sci. USA, Vol. 86, pp. 6553-6556 (1989); Lemaitre et al., Proc. Natl. Acad. Sci. USA, Vol. 84, pp. 648-652 (1987); and PCT Publication No. WO 88/09810 (1988)], hybridization-triggered cleavage agents [see, e.g., Krol et al., Bio Techniques, Vol. 6, pp. 958-976 (1988)] or intercalating agents [see, e.g., Zon, Pharm. Res., Vol. 5, No. 9, pp. 539-549 (1988)].

In a preferred aspect of the invention, an antisense oligonucleotide is provided, preferably as single-stranded DNA. The oligonucleotide may be modified at any position on its structure with constituents generally known in the art.

The antisense oligonucleotides may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w and 2,6-diaminopurine.

In another embodiment, the oligonucleotide comprises at least one modified sugar moiety selected from the group including, but not limited to, arabinose, 2-fluoroarabinose, xylulose and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester and a formacetal or analog thereof.

In yet another embodiment, the oligonucleotide is a 2-a-anomeric oligonucleotide. An a-anomeric oligonucleotide forms specific double-stranded hybrids with complementary RNA in which, contrary to the usual B-units, the strands run parallel to each other. See Gautier et al., Nucl. Acids Res., Vol. 15, pp. 6625-6641 (1987).

The oligonucleotide may be conjugated to another molecule, e.g., a peptide, hybridization triggered cross-linking agent, transport agent, hybridization-triggered cleavage agent, etc.

The antisense nucleic acids of the invention comprise a sequence complementary to at least a portion of a target RNA species. However, absolute complementary, although preferred, is not required. A sequence “complementary to at least a portion of an RNA”, as referred to herein, means a sequence having sufficient complementary to be able to hybridize with the RNA, forming a stable duplex; in the case of double-stranded antisense nucleic acids, a single strand of the duplex DNA may thus be tested or triplex formation may be assayed. The ability to hybridize will depend on both the degree of complementary and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

Oligonucleotides of the invention may be synthesized by standard methods known in the art, e.g., by use of an automated DNA synthesizer, such as are commercially-available from Biosearch, Applied Biosystems, etc. As examples, phosphorothioate oligonucleotides may be synthesized by the method of Stein et al., Nucl. Acids Res., Vol. 16, p. 3209 (1988), methylphosphonate oligonucleotides can be prepared by use of controlled pore glass polymer supports, etc. See Sarin et al., Proc. Natl. Acad. Sci. USA, Vol. 85, pp. 7448-7451 (1988). In another embodiment, the oligonucleotide is a 2′-0-methylribonucleotide [see Inoue et al., Nucl. Acids Res., Vol. 15, pp. 6131-6148 (1987)] or a chimeric RNA-DNA analog [see Inoue et al., FEBS Lett., Vol. 215, pp. 327-330 (1987)].

The synthesized antisense oligonucleotides can then be administered to a cell in a controlled or saturating manner. For example, the antisense oligonucleotides can be placed in the growth environment of the cell at controlled levels where they may be taken up by the cell. The uptake of the antisense oligonucleotides can be assisted by use of methods well-known in the art.

Antisense Molecules Expressed Intracellularly

In an alternative embodiment, the antisense nucleic acids of the invention are controllably expressed intracellularly by transcription from an exogenous sequence. If the expression is controlled to be at a high level, a saturating perturbation or modification results. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to, the SV40 early promoter region [see Bernoist and Chambon, Nature, Vol. 290, pp. 304-310 (1981)], the promoter contained in the 3′ long terminal repeat of Rous sarcoma virus [see Yamamoto et al., Cell, Vol. 22, pp. 787-797 (1980)], the herpes thymidine kinase promoter [see Wagner et al., Proc. Natl. Acad. Sci. USA, Vol. 78, pp. 1441-1445 (1981)], the regulatory sequences of the metallothionein gene, etc. [see Brinster et al., Nature, Vol. 296, pp. 39-42 (1982)].

Therefore, antisense nucleic acids can be routinely designed to target virtually any mRNA sequence, and a cell can be routinely transformed with or exposed to nucleic acids coding for such antisense sequences such that an effective and controllable or saturating amount of the antisense nucleic acid is expressed. Accordingly the translation of virtually any RNA species in a cell can be modified or perturbed.

RNA Aptamers

Finally, in a further embodiment, RNA aptamers can be introduced into or expressed in a cell. RNA aptamers are specific RNA ligands for proteins, such as for Tat and Rev RNA [see Good et al. (1997), supra] that can specifically inhibit their translation.

Methods of Modifying Protein Abundances

Methods of modifying protein abundances include, inter alia, those altering protein degradation rates and those using antibodies, which bind to proteins affecting abundances of activities of native target protein species. Increasing (or decreasing) the degradation rates of a protein species decreases (or increases) the abundance of that species. Methods for increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature, e.g., 37° C., and which is hidden to prevent rapid degradation at a lower temperature, e.g., 23° C. See Dohmen et al., Science, Vol. 263, pp. 1273-1276 (1994). Such an exemplary degron is Arg-DHFR^{ts}, a variant of murine dihydrofolate reductase in which the N-terminal Val is replaced by Arg and the Pro at position 66 is replaced with Leu. According to this method, e.g., a gene for a target protein, P, is replaced by standard gene targeting methods known in the art [see Lodish et al., Molecular Biology of the Cell, W. H. Freeman and Co., NY, especially Chapter 8 (1995)] with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow and active target protein levels are high. At higher temperatures (in the absence of methotrexate), lysines internal to Arg-DHFR^{ts} are exposed, ubiquitination of the fusion protein occurs, degradation is rapid and active target protein levels are low. This technique also permits controllable modification of degradation rates since heat activation of degradation is controllably blocked by exposure methotrexate. This method is adaptable to other N-terminal degrons which are responsive to other inducing factors, such as drugs and temperature changes.

Modifying Protein Activity With Antibodies

Target protein activities can also be decreased by (neutralizing) antibodies. By providing for controlled or saturating exposure to such antibodies, protein abundances/activities can be modified or perturbed in a controlled or saturating manner. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form. Alternately, antibodies may directly decrease protein activity by, e.g., interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below) and assaying the concentration of the wild-type amount or activities of the target protein by standard means, such as immunoassays, known in the art. The net activity of the wild-type form can be assayed by assay means appropriate to the known activity of the target protein.

Antibodies can be introduced into cells in numerous fashions, including, e.g., microinjection of antibodies into a cell [see Morgan et al., Immunol. Today, Vol. 9, pp. 84-86 (1988)] or transforming hybridoma mRNA encoding a desired antibody into a cell [see Burke et al., Cell, Vol. 36, pp. 847-858 (1984)]. In a further technique, recombinant antibodies can be engineering and ectopically expressed in a wide variety of non-lymphoid cell types to bind to target proteins, as well as to block target protein activities. See Biocca et al., Trends Cell Biol., Vol. 5, pp. 248-252 (1995). Expression of the antibody is preferably under control of a controllable promoter, such as the Tet promoter, or a constitutively active promoter (for production of saturating perturbations). A first step is the selection of a particular monoclonal antibody with appropriate specificity to the target protein (see below). Then sequences encoding the variable regions of the selected antibody can be cloned into various engineered antibody formats, including, e.g., whole antibody, Fab fragments, Fv fragments, single-chain Fv (ScFv) fragments (V_{H} and V_{L} regions united by a peptide linker), diabodies (two associated ScFv fragments with different specificities) and so forth. See Hayden et al., Curr. Opin. Immunol., Vol. 9, pp. 210-212 (1997). lntracellularly-expressed antibodies of the various formats can be targeted into cellular compartments, e.g., the cytoplasm, the nucleus, the mitochondria, etc., by expressing them as fusions with the various known intracellular leader sequences. See Bradbury et al., Antibody Engineering, Borrebaeck, Editor, Vol. 2, pp. 295-361, IRL Press (1995). In particular, the ScFv format appears to be particularly suitable for cytoplasmic targeting.

Antibody types include, but are not limited to, polyclonal, monoclonal, chimeric, single-chain, Fab fragments and an Fab expression library. Various procedures known in the art may be used for the production of polyclonal antibodies to a target protein. For production of the antibody, various host animals can be immunized by injection with the target protein, such host animals include, but are not limited to, rabbits, mice, rats, etc. Various adjuvants can be used to increase the immunological response, depending on the host species and include, but are not limited to, Freund's (complete and incomplete); mineral gels, such as aluminum hydroxide; surface active substances, such as lysolecithin, pluronic polyols, polyanions, peptides, oil emulsions, dinitrophenol; and potentially useful human adjuvants, such as Bacillus Calmette-Guerin (BCG) and corynebacterium parvum.

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein, Nature, Vol. 256, pp. 495-497 (1975), the trioma technique, the human B-cell hybridoma technique [see Kozbor et al., Immunol. Today, Vol. 4, p. 72 (1983)] and the EBV hybridoma technique to produce human monoclonal antibodies [see Cole et al., Monoclonal Antibodies and Cancer Therapy, Alan R. Liss, Inc., pp. 77-96 (1985)]. In an additional embodiment of the invention, monoclonal antibodies can be produced in germ-free animals utilizing recent technology. See PCT/US90/02545. According to the invention, human antibodies may be used and can be obtained by using human hybridomas [see Cote et al., Proc. Natl. Acad. Sci. USA, Vol. 80, pp. 2026-2030 (1983)], or by transforming human B cells with EBV virus in vitro [see Cole et al. (1985), supra]. In fact, according to the invention, techniques developed for the production of “chimeric antibodies” [see Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452454 (1985)] by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display. See Marks et al., J. Biol. Chem., Vol. 267, pp. 16007-16010 (1992). Using this technique, libraries of up to 10¹² different antibodies have been expressed on the surface of fd filamentous phage, creating a “single pot” in vitro immune system of antibodies available for the selection of monoclonal antibodies. See Griffiths et al., EMBO J., Vol. 13, pp. 3245-3260 (1994). Selection of antibodies from such libraries can be done by techniques known in the art, including contacting the phage to immobilized target protein, selecting and cloning phage bound to the target and subcloning the sequences encoding the antibody variable regions into an appropriate vector expressing a desired antibody format.

According to the invention, techniques described for the production of single-chain antibodies (see U.S. Pat. No. 4,946,778) can be adapted to produce single-chain antibodies specific to the target protein. An additional embodiment of the invention utilizes the techniques described for the construction of Fab expression libraries [see Huse et al., Science, Vol. 246, pp. 1275-1281 (1989)] to allow rapid and easy identification of monoclonal Fab fragments with the desired specificity for the target protein.

Antibody fragments that contain the idiotypes of the target protein can be generated by techniques known in the art. For example, such fragments include, but are not limited to, the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent and Fv fragments.

In the production of antibodies, screening for the desired antibody can be accomplished by techniques known in the art, e.g., ELISA. To select antibodies specific to a target protein, one may assay generated hybridomas or a phage display antibody library for an antibody that binds to the target protein.

Methods of Modifying Protein Activities

Methods of directly modifying protein activities include, inter alia, dominant negative mutations, specific drugs or chemical moieties and also the use of antibodies, as previously discussed.

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target. See Hershkowitz, Nature, Vol. 329, pp. 219-222 (1987). In the case of active monomeric forms, over expression of an inactive form can cause competition for natural substrates or ligands sufficient to significantly reduce net activity of the target protein. Such over expression can be achieved by, e.g., associating a promoter, preferably a controllable or inducible promoter, or also a constitutively expressed promoter, of increased activity with the mutant gene. Alternatively, changes to active site residues can be made so that a virtually irreversible association occurs with the target ligand. Such can be achieved with certain tyrosine kinases by careful replacement of active site serine residues. See Perimutter et al., Curr. Opin. Immunol., Vol. 8, pp. 285-290 (1996).

In the case of active multimeric forms, several strategies can guide selection of a dominant negative mutant. Multimeric activity can be decreased in a controlled or saturating manner by expression of genes coding exogenous protein fragments that bind to multimeric association domains and prevent multimer formation. Alternatively, controllable or saturating over-expression of an inactive protein unit of a particular type can tie up wild-type active units in inactive multimers, and thereby decrease multimeric activity. See Nocka et al., EMBO J., Vol. 9, pp. 1805-1813 (1990). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture and so forth, are typically composed of associations of many subunits of a few types. These structures are often highly sensitive to disruption by inclusion of a few monomeric units with structural defects. Such mutant monomers disrupt the relevant protein activities and can be expressed in a cell in a controlled or saturating manner.

In addition to dominant negative mutations, mutant target proteins that are sensitive to temperature (or other exogenous factors) can be found by mutagenesis and screening procedures that are well-known in the art.

Also, one of skill in the art will appreciate that expression of antibodies binding and inhibiting a target protein can be employed as another dominant negative strategy.

Modifying Proteins with Small Molecule Drugs

Finally, activities of certain target proteins can be modified or perturbed in a controlled or a saturating manner by exposure to exogenous drugs or ligands. Since the methods of this invention are often applied to testing or confirming the usefulness of various drugs to treat cancer, drug exposure is an important method of modifying/perturbing cellular constituents, both mRNAs and expressed proteins. In a preferred embodiment, input cellular constituents are perturbed either by drug exposure or genetic manipulation, such as gene deletion or knockout; and system responses are measured by gene expression technologies, such as hybridization to gene transcript arrays (described in the following).

In a preferable case, a drug is known that interacts with only one target protein in the cell and alters the activity of only that one target protein, either increasing or decreasing the activity. Graded exposure of a cell to varying amounts of that drug thereby causes graded perturbations of network models having that target protein as an input. Saturating exposure causes saturating modification/perturbation. For example, Cyclosporin A is a very specific regulator of the calcineurin protein, acting via a complex with cyclophilin. A titration series of Cyclosporin A therefore can be used to generate any desired amount of inhibition of the calcineurin protein. Alternately, saturating exposure to Cyclosporin A will maximally inhibit the calcineurin protein.

Measurement Methods

The experimental methods of this invention depend on measurements of cellular constituents. The cellular constituents measured can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, e.g., in which the activities of one or more proteins are measured along with the RNA abundances (gene expressions) of other cellular constituents. This section describes exemplary methods for measuring the cellular constituents in drug or pathway responses. This invention is adaptable to other methods of such measurement.

Preferably, in this invention the transcriptional state of the other cellular constituents are measured. The transcriptional state can be measured by techniques of hybridization to arrays of nucleic acid or nucleic acid mimic probes, described in the next subsection, or by other gene expression technologies, described in the subsequent subsection. However measured, the result is data including values representing mRNA abundance and/or ratios, which usually reflect DNA expression ratios (in the absence of differences in RNA degradation rates).

In various alternative embodiments of the present invention, aspects of the biological state other than the transcriptional state, such as the translational state, the activity state or mixed aspects can be measured.

In all embodiments, measurements of the cellular constituents should be made in a manner that is relatively independent of when the measurement are made.

Transcriptional State Measurement

Preferably, measurement of the transcriptional state is made by hybridization to transcript arrays, which are described in this subsection. Certain other methods of transcriptional state measurement are described later in this subsection.

Transcript Arrays Generally

In a preferred embodiment the present invention makes use of “transcript arrays”, also called herein “microarrays”. Transcript arrays can be employed for analyzing the transcriptional state in a cell, and especially for measuring the transcriptional states of cancer cells.

In one embodiment, transcript arrays are produced by hybridizing detectably-labeled polynucleotides representing the mRNA transcripts present in a cell, e.g., fluorescently-labeled cDNA synthesized from total cell mRNA, to a microarray. A microarray is a surface with an ordered array of binding, e.g., hybridization, sites for products of many of the genes in the genome of a cell or organism, preferably most or almost all of the genes. Microarrays can be made in a number of ways, of which several are described below. However produced, microarrays share certain characteristics. The arrays are reproducible, allowing multiple copies of a given array to be produced and easily compared with each other. Preferably the microarrays are small, usually smaller than 5 cm² and they are made from materials that are stable under binding, e.g. nucleic acid hybridization, conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. In a specific embodiment, positionally-addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled, e.g., with a fluorophore, cDNA complementary to the total cellular mRNA is hybridized to a microarray, the site on the array corresponding to a gene, i.e., capable of specifically binding the product of the gene, that is not transcribed in the cell will have little or no signal, e.g., fluorescent signal, and a gene for which the encoded mRNA is prevalent will have a relatively strong signal.

Preparation of Microarrays

Microarrays are known in the art and consist of a surface to which probes that correspond in sequence to gene products, e.g., cDNAs, mRNAs, cRNAs, polypeptides and fragments thereof, can be specifically hybridized or bound at a known position. In one embodiment, the microarray is an array, i.e., a matrix, in which each position represents a discrete binding site for a product encoded by a gene, e.g., a protein or RNA, and in which binding sites are present for products of most or almost all of the genes in the organism's genome. In a preferred embodiment, the “binding site”, hereinafter “site”, is a nucleic acid or nucleic acid analogue to which a particular cognate cDNA can specifically hybridize. The nucleic acid or analogue of the binding site can be, e.g., a synthetic oligomer, a full-length cDNA, a less than full-length cDNA or a gene fragment.

Although in a preferred embodiment the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to testing and confirming a biological network model of interest. A “gene” is identified as an open reading frame (ORF) of preferably at least 50, 75 or 99 amino acids from which a mRNA is transcribed in the organism, e.g., if a single cell, or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6,275 ORFs longer than 99 amino acids. Analysis of these ORFs indicates that there are 5,885 ORFs that are likely to specify protein products. See Goffeau et al., Science, Vol. 274, pp. 546-567 (1996), which is incorporated by reference in its entirety for all purposes. In contrast, the human genome is estimated to contain approximately 10⁵ genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., PCR amplification of gene segments from genomic DNA, cDNA, e.g., by RT-PCR, or cloned sequences. PCR primers are chosen, based on the known sequence of the genes or cDNA, that result in amplification of unique fragments, i.e., fragments that do not share more than 10 bases of contiguous identical sequence with any other fragment on the microarray. Computer programs are useful in the design of primers with the required specificity and optimal amplification properties. See, e.g., Oligo pI version 5.0, National Biosciences. In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well-known and are described, e.g., in Innis et al., eds., PCR Protocols: A Guide to Methods and Applications, Academic Press Inc., San Diego, Calif. (1990), which is incorporated by reference in its entirety for all purposes. It will be apparent that computer-controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries. See Froehler et al., Nucleic Acid Res., Vol. 14, pp. 5399-5407 (1986); and McBride et al., Tetrahedron Lett., Vol. 24, pp. 245-248 (1983). Synthetic sequences are between about 15 bases and about 500 bases in length, more typically between about 20 bases and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid. See, e.g., Egholm et al., Nature, Vol. 365, pp. 566-568 (1993); and also U.S. Pat. No. 5,539,083.

In an alternative embodiment, the binding (hybridization) sites are made from plasmid or phage clones of genes, cDNAs, e.g., expressed sequence tags, or inserts therefrom. See Nguyen et al., Genomics, Vol. 29, pp. 207-209 (1995). In yet another embodiment, the polynucleotide of the binding sites is RNA.

Attaching Nucleic Acids to the Solid Surface

The nucleic acid or analogue are attached to a solid support, which may be made from glass, plastic, e.g., polypropylene and nylon, polyacrylamide, nitrocellulose or other materials. A preferred method for attaching the nucleic acids to a surface is by printing on glass plates, as is described generally by Schena et al., Science, Vol. 270, pp. 467-470 (1995). This method is especially useful for preparing microarrays of cDNA. See, also, DeRisi et al., Nat. Genet.,Vol. 14, pp. 457-460 (1996); Shalon et al., Genome Res, Vol. 6, pp. 639-645 (1996); and Schena et al., Proc. Natl. Acad. Sci. USA, Vol. 93, pp. 10539-11286 (1995). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ [see Fodor et al., Science, Vol. 251, pp. 767-773 (1991); Pease et al., Proc. Natl. Acad. Sci. USA, Vol. 91, No. 11, pp. 5022-5026 (1994); Lockhart et al. Nat. Biotechnol., Vol. 14, p. 1675 (1996); and U.S. Pat. Nos. 5,578,832; 5,556,752; and 5,510,270, each of which is incorporated by reference in its entirety for all purposes] or other methods for rapid synthesis and deposition of defined oligonucleotides [see Blanchard et al., Biosens. Bioelectron., Vol. 11, pp. 687-690 (1996)]. When these methods are used, oligonucleotides, e.g., 20 mers, of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking, may also be used. See Maskos and Southern, Nucleic Acids Res., Vol. 20, pp. 1679-1684 (1992). In principal, any type of array, e.g., dot blots on a nylon hybridization membrane [see Sambrook et al., Molecular Cloning—A Laboratory Manual, 2^{nd} Edition, Vols. 1-3, Cold Spring Harbor Laboratory, Cold Spring Harbor, N.Y. (1989), which is incorporated in its entirety for all purposes], could be used, although, as will be recognized by those of skill in the art, very small arrays will be preferred because hybridization volumes will be smaller.

Generating Labeled Probes

Methods for preparing total and poly(A)⁺ RNA are well-known and are described generally in Sambrook et al. (1989), supra. In one embodiment, RNA is extracted from cells of the various types of interest in this invention using guanidinium thiocyanate lysis followed by CsCl centrifugation. See Chirgwin et al., Biochemistry, Vol. 18, pp. 5294-5299 (1979). Poly(A)⁺ RNA is selected by selection with oligo-dT cellulose. See Sambrook et al. (1989), supra. Cells of interest include wild-type cells, drug-exposed wild-type cells, cells with modified/perturbed cellular constituent(s), and drug-exposed cells with modified/perturbed cellular constituent(s).

Labeled cDNA is prepared from mRNA by oligo dT-primed or random-primed reverse transcription, both of which are well-known in the art. See, e.g., Klug and Berger, Methods Enzymol., Vol. 152, pp. 316-325 (1987). Reverse transcription may be carried out in the presence of a dNTP conjugated to a detectable label, most preferably a fluorescently-labeled dNTP. Alternatively, isolated mRNA can be converted to labeled antisense RNA synthesized by in vitro transcription of double-stranded cDNA in the presence of labeled dNTPs. See Lockhart et al. (1996), supra, which is incorporated by reference in its entirety for all purposes. In alternative embodiments, the cDNA or RNA probe can be synthesized in the absence of detectable label and may be labeled subsequently, e.g., by incorporating biotinylated dNTPs or rNTP, or some similar means, e.g., photo-cross-linking a psoralen derivative of biotin to RNAs, followed by addition of labeled streptavidin, e.g., phycoerythrin-conjugated streptavidin or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others. See, e.g., Kricka, Nonisotopic DNA Probe Techniques, Academic Press, San Diego, Calif. (1992). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used. See Zhao et al., Gene, Vol. 156, p. 207 (1995); and Pietu et al., Genome Res., Vol. 6, p. 492 (1996). However, because of scattering of radioactive particles, and the consequent requirement for widely-spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides, e.g., 0.1 mM Rhodamine 110 UTP (Perken Elmer Cetus) or 0.1 mM Cy3 dUTP (Amersham), with reverse transcriptase, e.g., SuperScript.TM.II, LTI Inc., at 42° C. for 60 minutes.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is ≦25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls. See, e.g., Shalon et al. (1996), supra; and Chee et al., supra.

Optimal hybridization conditions will depend on the length, e.g., oligomer vs. polynucleotide >200 bases; and type, e.g., RNA, DNA and PNA, of labeled probe and immobilized polynucleotide or oligonucleotide. General parameters for specific, i.e., stringent, hybridization conditions for nucleic acids are described in Sambrook et al. (1996), supra; and Ausubel et al., Current Protocols in Molecular Biology, Greene Publishing and Wiley-Interscience, NY (1987), which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low-stringency wash buffer (1×SSC plus 0.2% SDS) followed by 10 minutes at 25° C. in high-stringency wash buffer (0.1×SSC plus 0.2% SDS). See Shena et al., Proc. Natl. Acad. Sci. USA, Vol. 93, p. 10614 (1996). Useful hybridization conditions are also provided. See, e.g., Tijessen, Hybridization With Nucleic Acid Probes, Elsevier Science Publishers B. V. (1993); and Kricka (1992), supra.

Signal Detection and Data Analysis

When fluorescently-labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously. See Shalon et al. (1996), supra, which is incorporated by reference in its entirety for all purposes. In a preferred embodiment, the arrays are scanned with a laser fluorescent scanner with a computer-controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al. (1996), supra and in other references cited herein. Alternatively, the fiber-optic bundle described by Ferguson et al., Nat Biotechnol., Vol. 14, pp. 1681-1684 (1996), may be used to monitor mRNA abundance levels at a large number of sites simultaneously.

Signals are recorded and, in a preferred embodiment, analyzed by computer, e.g., using a 12-bit analog to digital board. In one embodiment the scanned image is de-speckled using a graphics program, e.g., Hijaak Graphics Suite, and then analyzed using an image gridding program that creates a spreadsheet of the average hybridization at each wavelength at each site. If necessary, an experimentally determined correction for “cross talk” (or overlap) between the channels for the two fluorophores may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion or any other tested event.

Preferably, in addition to identifying a perturbation as positive or negative, it is advantageous to determine the magnitude of the perturbation. This can be carried out by methods that will be readily apparent to those of skill in the art.

Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers [see, e.g., EP 0 534858 A1 (1992), Zabeau et al.], or methods selecting restriction fragments with sites closest to a defined mRNA end [see, e.g., Prashar et al., Proc. Natl. Acad. Sci. USA, Vol. 93, pp. 659-663 (1996)]. Other methods statistically sample cDNA pools, such as by sequencing sufficient bases, e.g., 20-50 bases, in each of multiple cDNAs to identify each cDNA, or by sequencing short tags, e.g., 9-10 bases, which are generated at known positions relative to a defined mRNA end pathway pattern. See, e.g., Velculescu, Science, Vol. 270, pp. 484-487 (1995).

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Measurement of the translational state may be performed according to several methods. For example, whole genome monitoring of protein, i.e., the “proteome” [see Goffeau et al. (1996), supra], can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well-known. See, e.g., Harlow and Lane, Antibodies: A Laboratory Manual, Cold Spring Harbor, NY (1988), which is incorporated in its entirety for all purposes. In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., Gel Electrophoresis of Proteins: A Practical Approach, IRL Press, NY (1990); Shevchenko et al., Proc. Natl. Acad. Sci. USA, Vol. 93, pp. 1440-1445 (1996); Sagliocco et al., Yeast, Vol. 12, pp. 1519-1533 (1996); Lander, Science, Vol. 274, pp. 536-539 (1996). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells, e.g., in yeast; exposed to a drug or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical or physical means appropriate to the particular activity being characterized. Where the activity involves a chemical transformation, the cellular protein can be contacted with the natural substrates and the rate of transformation measured. Where the activity involves association in multimeric units, e.g., association of an activated DNA-binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

Computer Implementations

In a preferred embodiment, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with a main memory. For example computer system can be an Intel Pentium based processor of 200 Mhz or greater clock rate and with 32 MB or more of main memory.

The external components include mass data storage. This mass storage can be one or more hard disks, which are typically packaged together with the processor and memory. Typically, such hard disks provide for at least 1 GB of storage. Other external components include user interface device, which can be a monitor and keyboards, together with pointing device, which can be a “mouse” or other graphic input devices. Typically, the computer system is also linked to other local computer systems, remote computer systems, or wide area communication networks, such as the internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. Alternatively, the software components may be stored on removable media such as floppy disks or CD-ROM (not illustrated). The software component represents the operating system, which is responsible for managing the computer system and its network interconnections. This operating system can be, e.g., of the Microsoft Windows family, such as Windows 95, Windows 98 or Windows NT; or a Unix operating system, such as Sun Solaris. Software include common languages and functions conveniently present on this system to assist programs implementing the methods specific to this invention. Languages that can be used to program the analytic methods of this invention include C, C++ or, less preferably, JAVA. Most preferably, the methods of this invention are programmed in mathematical software packages which allow symbolic entry of equations and high-level specification of processing, including algorithms to be used, thereby freeing a user of the need to procedurally program individual equations or algorithms. Such packages include, e.g., Matlab from Mathworks (Natick, Mass.), Mathematica from Wolfram Research (Champaign, Ill.) and MathCAD from Mathsoft (Cambridge, Mass.).

In preferred embodiments, the analytic software component actually comprises separate software components which interact with each other. Analytic software represents a database containing all data necessary for the operation of the system. Such data will generally include, but is not necessarily limited to, results of prior experiments, genome data, experimental procedures and cost and other information which will be apparent to those skilled in the art. Analytic software includes a data reduction and computation component comprising one or more programs which execute the analytic methods of the invention.

Analytic software also includes a user interface which provides a user of the computer system with control and input of test network models, and, optionally, experimental data. The user interface may comprise a drag-and-drop interface for specifying hypotheses to the system. The user interface may also comprise means for loading experimental data from the mass storage component, e.g., the hard drive; from removable media, e.g., floppy disks or CD-ROM; or from a different computer system communicating with the instant system over a network, e.g., a local area network, or a wide area communication network, such as the internet.

Alternative systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

TABLE 2
Quantification of Purified Total RNA and cRNA

| | Weight | RNA Yield | cRNA Yield | cRNA Used |
|---|---|---|---|---|
| Sample code | (mg) | (μg) | (μg) | (μg) |
| p2437 | — | — | 30.0 | 15.0 |
| p2709 | — | — | 36.3 | 15.0 |
| p5720 | — | — | 29.0 | 15.0 |
| p5721 | — | — | 21.5 | 15.0 |
| p6166 | 56.3 | 45.0 | 58.8 | 15.0 |
| p6167 | 80.0 | 9.4 | 47.1 | 15.0 |
| p6168 | 30.0 | 7.8 | 42.4 | 15.0 |
| p6169 | 65.6 | 7.4 | 48.4 | 15.0 |
| p6170 | 51.9 | 40.9 | 13.2 | 13.2 |
| p6171 | 90.0 | 16.1 | 52.9 | 15.0 |
| p6172 | 47.0 | 9.1 | 29.6 | 15.0 |
| p6173 | 88.0 | 15.7 | 31.5 | 15.0 |
| p6174 | 86.1 | 26.6 | 13.9 | 13.9 |
| p6175 | 38.0 | 0.8 | — | — |
| p6176 | 80.2 | 57.1 | 14.1 | 14.1 |
| p6177 | 71.0 | 33.2 | 19.6 | 15.0 |
| p6178 | 77.0 | 16.4 | 35.4 | 15.0 |
| p6179 | 47.4 | 1.1 | — | — |
| p6180 | 60.5 | 89.9 | 36.4 | 15.0 |
| p6181 | 24.2 | 8.2 | 19.6 | 15.0 |
| p6182 | 70.0 | 63.7 | 15.0 | 15.0 |
| p6183 | 58.6 | 7.1 | 9.8 | — |
| p6184 | 84.5 | 38.1 | 12.8 | 12.8 |
| p6185 | 90.0 | 6.7 | 25.2 | 15.0 |

TABLE 3
Summary of Experiment QC

| | | | | % of | | | Laboratory |
|---|---|---|---|---|---|---|---|
| | Chip | | Scaling | Genes | GAPDH | B-actin | (Where Profiles |
| Sample Code | Designation | Background | Factor | Present | 3′/5′ | 3′/5′ | Were Generated) |
| p2437 | p2437e | 893 | 0.18 | 52.46 | 2.04 | 4.51 | PG |
| p2709 | p2709e | 776 | 0.35 | 46.26 | 2.09 | 1.08 | PG |
| p5720 | p5720-2ee | 74 | 3.05 | 48.09 | 1.63 | 1.14 | PG |
| p5721 | p5721-2ee | 55 | 3.24 | 47.90 | 5.09 | 6.91 | PG |
| p6166 | p6166ee | 60 | 1.72 | 50.27 | 1.55 | 1.38 | PG |
| p6167 | p6167ee | 48 | 2.71 | 53.12 | 2.35 | 3.03 | PG |
| p6168 | p6168ee | 46 | 2.71 | 53.81 | 2.76 | 2.54 | PG |
| p6169 | p6169ee | 59 | 14.62 | 22.55 | 5.36 | 10.38 | PG |
| p6170 | p6170ee | 55 | 2.14 | 38.24 | 1.60 | 2.06 | PG |
| p6171 | p6171ee | 47 | 2.27 | 55.19 | 2.47 | 2.99 | PG |
| p6172 | p6172ee | 55 | 3.73 | 46.84 | 4.43 | 4.53 | PG |
| p6173 | p6173ee | 61 | 2.43 | 51.01 | 7.62 | 5.99 | PG |
| p6174 | p6174ee | 82 | 1.19 | 53.04 | 1.38 | 1.80 | PG |
| p6176 | p6176ee | 60 | 2.28 | 48.59 | 1.49 | 2.44 | PG |
| p6177 | p6177ee | 97 | 2.31 | 46.64 | 1.73 | 2.06 | PG |
| p6178 | p6178ee | 73 | 2.65 | 41.76 | 1.65 | 1.81 | PG |
| p6180 | p6180ee | 43 | 10.59 | 27.84 | 4.46 | 13.90 | PG |
| p6181 | p6181ee | 65 | 2.60 | 46.08 | 1.93 | 1.94 | PG |
| p6182 | p6182ee | 43 | 5.39 | 37.16 | 4.48 | 10.86 | PG |
| p6184 | p6184ee | 59 | 3.46 | 49.31 | 2.44 | 2.36 | PG |
| p6185 | p6185ee | 49 | 8.18 | 40.63 | 3.67 | 4.51 | PG |
| OVR1T | h9lms01031301 | 122 | 1.55 | 43.62 | 1.62 | 1.61 | GNF |
| OVR2T | h9lms01030602 | 119 | 1.70 | 43.16 | 1.69 | 1.91 | GNF |
| OVR5T | h9lms01030603 | 86 | 0.92 | 52.46 | 1.30 | 2.33 | GNF |
| OVR8T | h9lms01030604 | 76 | 1.27 | 50.49 | 1.65 | 1.93 | GNF |
| OVR10T | h9lms01030714 | 80 | 1.12 | 49.86 | 1.15 | 2.41 | GNF |
| OVR11T | h9lms01030605 | 71 | 1.40 | 47.29 | 1.90 | 3.34 | GNF |
| OVR12T | h9lms01030606 | 78 | 1.69 | 43.68 | 1.57 | 1.68 | GNF |
| OVR13T | h9lms01030607 | 75 | 0.75 | 51.90 | 1.26 | 1.80 | GNF |
| OVR16T | h9lms01030501 | 168 | 0.86 | 39.03 | 1.68 | 2.95 | GNF |
| OVR19T | h9lms01030502 | 80 | 1.22 | 43.54 | 1.49 | 2.17 | GNF |
| OVR22T | h9lms01030608 | 83 | 1.22 | 43.54 | 1.61 | 2.82 | GNF |
| OVR26T | h9lms01030609 | 85 | 1.01 | 46.21 | 1.38 | 2.66 | GNF |
| OVR27T | h9lms01030503 | 104 | 0.72 | 49.99 | 1.40 | 2.02 | GNF |
| OVR28T | h9lms01030504 | 140 | 1.40 | 35.17 | 2.80 | 6.37 | GNF |
| OVR102N | h9lms00102618 | 130 | 2.02 | 33.01 | 3.49 | 5.60 | GNF |
| OVR278EN | h9lms11030505 | 59 | 3.70 | 32.96 | 2.20 | 3.60 | GNF |
| OVR278SN | h9lms01030715 | 57 | 3.12 | 36.10 | 1.87 | 2.53 | GNF |
| HUOVR | h9lms00102622 | 68 | 1.16 | 51.60 | 1.43 | 2.72 | GNF |

TABLE 4
Comparison of Predicted Vs. Observed Status of 18 Ovarian Test Samples
With a 28 Probe Sets Expression Profile

| Biopsy status | | | |
|---|---|---|---|
| Obs. (Exp.) | r ≦ 0.920 | r > 0.920 | Total |
| Normal | 1 (4.44) | 7 (3.56) | 8 |
| Tumor | 9 (5.56) | 1 (4.44) | 10 |
| Total | 10 | 8 | 18 |

Note:
The number of observations is shown for each group of samples, with the value expected under random association in parentheses.
“r” = the PCC value of the 28 probe set profile of a biopsy sample with the mean Normal profile.
OR = 63 (95% CI: 3.3-1194.7),
p = 0.0029

TABLE 5
Comparison of Predicted Vs. Observed Status of 36 Ovarian
Samples With a 28, 32 or 42 Probe Sets Expression Profile

| Number of Probe Sets | 28 | 32 | 42 |
|---|---|---|---|
| Correlation threshold used | 0.914 | 0.933 | 0.851 |
| OR | 253 | 392 | 216 |
| 95% CI | 14.4-4432.9 | 14.8-10339.6 | 9.6-4882.5 |
| p-value (Fischer's | 2.3 × 10⁻⁷ | 1.0 × 10⁻⁸ | 1.3 × 10⁻⁷ |
| exact test) | | | |

TABLE 6
List of Genes Up-Regulated in Ovarian Tumors
Absolute CC values are shown for expression levels analyzed in the 18 test
samples only (R1) or in all 36 samples (R2).

| Probe Set | Gene | | Cytogenetic | | |
|---|---|---|---|---|---|
| Name | Symbol | Description | Location | R1 | R2 |
| 40145_at | TOP2A | Topoisomerase (DNA) II alpha | 17q21-q22 | 0.643 | 0.636 |
| | | (170 kD) | | | |
| 39109_at | C20ORF1 | Chromosome 20 ORF 1 | 20q11.2 | 0.623 | 0.564 |
| 39829_at | ARL7 | ADP-ribosylation factor-like 7 | 2q37.2 | 0.618 | 0.656 |
| 37985_at | LMNB1 | Lamin B1 | 5q23.3-q31.1 | 0.565 | 0.608 |
| 2092_s_at | SPP1 | Secreted phosphoprotein 1 | 4q21-q25 | 0.560 | 0.679 |
| | | (osteopontin) | | | |
| 38116_at | KIAA0101 | KIAA0101 gene product | 15q22.1 | 0.556 | 0.595 |
| 34259_at | KIAA0664 | KIAA0664 protein | 17p13.3 | 0.544 | 0.541 |
| 35276_at | CLDN4 | Claudin 4 | 7q11.23 | 0.471 | 0.575 |
| 149_at | DDXL | Nuclear RNA helicase | 19p13.13 | 0.457 | 0.544 |
| 37131_at | KLK8 | Kaliikrein 8 (neuropsin/ovasin) | 19q13.3-q13.4 | 0.443 | 0.585 |

TABLE 7
Functional Categories of the Most Differentially-Expressed Genes in
Ovarian Cancer

| Categories | Top 28 Probe Sets | Top 100 Probe Sets* |
|---|---|---|
| Cell cycle regulation | 8 | 13* |
| Growth differentiation | | |
| and cell death | | |
| Tumor suppression | | |
| Transcription regulation | 1 | 17* |
| Signal transduction | 3 | 9* |
| Metabolic enzymes | 5 | 9 |
| Cytoskeletal proteins | 1 | 5 |
| Extracellular matrix | 1 | 6 |
| Others | 1 | 14 |
| Unknown | 8 | 27 |

*For 5 genes (GPRK5, IGFBP5, IRS1, ITPR1 and RBPMS) similar results were obtained with 2 different probe sets.

TABLE 8
List of Genes Down-Regulated in Ovarian Tumors Functional Category

| | | | | | | |
|---|---|---|---|---|---|---|
| Probe Set | Gene | | Cytogenetic | | | |
| Name | Symbol | Description | Location | R1 | R2 | Rank |

Cell cycle regulation, growth differentiation, cell death, tumor suppression

| | | | | | | |
|---|---|---|---|---|---|---|
| 38120_at | PKD2 | Polycystic kidney | 4q21-q23 | 0.852 | 0.768 | 25 |
| | | disease 2 | | | | |
| 34257_at | AIP1 | Atrophin-1 interacting | 7q21 | 0.846 | 0.786 | 13 |
| | | protein 1 | | | | |
| 38650_at | IGFBP5 | Insulin-like growth factor | 2q33-34 | 0.840 | 0.748 | |
| | | binding protein 5 | | | | |
| 1396_at | IGFBP5 | Insulin-like growth factor | 2q33-q36 | 0.821 | 0.756 | |
| | | binding protein 5 | | | | |
| 1897_at | TGFβR3 | Transforming growth | 1p33-p32 | 0.809 | 0.770 | 23 |
| | | factor, beta receptor III | | | | |
| 36073_at | NDN | Necdin homolog | 15q11.2-q12 | 0.804 | 0.774 | 21 |
| 36160_s_at | PTPRN2 | Protein tyrosine | 7q36 | 0.788 | 0.846 | 3 |
| | | phosphatase, receptor | | | | |
| | | type, N polypeptide 2 | | | | |
| 37643_at | TNFRSF6 | Tumor necrosis factor | 10q24.1 | 0.779 | 0.716 | |
| | | receptor superfamily, | | | | |
| | | member 6 | | | | |
| 1640_at | ST13 | Suppression of | 22q13.2 | 0.769 | 0.734 | |
| | | tumorigenicity 13 | | | | |
| 35234_at | RECK | Reversion-inducing- | 9p13-p12 | 0.741 | 0.634 | |
| | | cysteine-rich protein with | | | | |
| | | kazal motifs | | | | |
| 1731_at | PDGFRα | Platelet-derived growth | 4q11-q13 | 0.739 | 0.619 | |
| | | factor receptor, alpha | | | | |
| | | polypeptide | | | | |
| 1761_at | PDGFRL | Platelet-derived growth | 8p22-p21.3 | 0.736 | 0.789 | 10 |
| | | factor receptor-like | | | | |
| 1327_s_at | MAP3K5 | Mitogen-activated protein | 6q22.33 | 0.729 | 0.626 | |
| | | kinase kinase kinase 5 | | | | |
| 39701_at | PEG3 | Paternally expressed 3 | 19q13.4 | 0.724 | 0.611 | |
| 36948_at | CRI1 | CREBBP/EP300 inhibitory | 15q21.1-q21.2 | 0.703 | 0.766 | 27* |
| | | protein 1 | | | | |
| 32668_at | SSBP2 | Single-stranded DNA | 5q14.1 | 0.503 | 0.809 | 6* |
| | | binding protein 2 | | | | |

Transcriptional regulation

| | | | | | | |
|---|---|---|---|---|---|---|
| 1577_at | AR | Androgen receptor | Xq11.2-q12 | 0.736 | 0.767 | 26 |
| 32664_at | RNASE4 | Ribonuclease, RNase A | 14q11.1 | 0.848 | 0.749 | |
| | | family, 4 | | | | |
| 38439_at | NFE2L1 | Nuclear factor (erythroid- | 17q21.3 | 0.829 | 0.736 | |
| | | derived 2)-like 1 | | | | |
| 38047_at | RBPMS | RNA-binding protein gene | 8p12-p11 | 0.822 | 0.756 | |
| | | with multiple splicing | | | | |
| 40775_at | ITM2A | Integral membrane | Xq13.3-Xq21.2 | 0.818 | 0.666 | |
| | | protein 2A | | | | |
| 34163_g_at | RBPMS | RNA-binding protein gene | 8p12-p11 | 0.776 | 0.709 | |
| | | with multiple splicing | | | | |
| 40570_at | FOXO1A | Forkhead box O1A | 13q14.1 | 0.775 | 0.668 | |
| | | (rhabdomyosarcoma) | | | | |
| 35681_r_at | ZFHX1B | Zinc finger homeobox 1b | 2q22 | 0.769 | 0.644 | |
| 40202_at | BTEB1 | Basic transcription | 9q13 | 0.754 | 0.606 | |
| | | element binding protein 1 | | | | |
| 41505_r_at | MAF | v-maf musculoaponeurotic | 16q22-q23 | 0.738 | 0.638 | |
| | | fibrosarcoma oncogene | | | | |
| | | homolog | | | | |
| 34355_at | MECP2 | Methyl CpG binding | Xq28 | 0.728 | 0.662 | |
| | | protein 2 (Rett syndrome) | | | | |
| 32259_at | EZH1 | Enhancer of zeste | 17q21.1-q21.3 | 0.728 | 0.611 | |
| | | homolog 1 | | | | |
| 41000_at | CHES1 | Checkpoint suppressor 1 | 14q24.3-q31 | 0.727 | 0.677 | |
| 34740_at | FOXO3A | Forkhead box O3A | 6q21 | 0.723 | 0.520 | |
| 39243_s_at | PSIP2 | PC4 and SFRS1 | 9p22.1 | 0.721 | 0.702 | |
| | | interacting protein 2 | | | | |

Signal transduction

| | | | | | | |
|---|---|---|---|---|---|---|
| 755_at | ITPR1 | Inositol 1,4,5-triphosphate | 3p26-p25 | 0.809 | 0.822 | 4 |
| | | receptor, type 1 | | | | |
| 38176_at | GNβ5 | Guanine nucleotide | 15q15.3 | 0.805 | 0.729 | |
| | | binding protein (G protein), | | | | |
| | | beta 5 | | | | |
| 39397_at | NR2F2 | Nuclear receptor subfamily | 15q26 | 0.768 | 0.700 | |
| | | 2, group F, member 2 | | | | |
| 872_i_at | IRS1 | Insulin receptor | 2q36 | 0.756 | 0.770 | 24 |
| | | substrate 1 | | | | |
| 40994_at | GPRK5 | G protein-coupled receptor | 10q24-qter | 0.749 | 0.627 | |
| | | kinase 5 | | | | |
| 37908_at | GNG11 | Guanine nucleotide | 7q31-q32 | 0.748 | 0.557 | |
| | | binding protein 11 | | | | |
| 41049_at | IRS1 | Insulin receptor | 2q36 | 0.744 | 0.694 | |
| | | substrate 1 | | | | |
| 32778_at | ITPR1 | Inositol 1,4,5-triphosphate | 3p26-p25 | 0.743 | 0.689 | |
| | | receptor, type 1 | | | | |
| 1135_at | GPRK5 | G protein-coupled receptor | 10q24-qter | 0.732 | 0.652 | |
| | | kinase 5 | | | | |
| 34877_at | JAK1 | Janus kinase 1 | 1p32.3-p31.3 | 0.720 | 0.671 | |
| 41796_at | PLCL2 | Phospholipase C-like 2 | 3p24.3 | 0.689 | 0.779 | 17* |

Metabolic enzymes

| | | | | | | |
|---|---|---|---|---|---|---|
| 32764_at | PHYH | Phytanoyl-CoA | 10pter-p11.2 | 0.803 | 0.691 | |
| | | hydroxylase (Refsum | | | | |
| | | disease) | | | | |
| 37628_at | MAOB | Monoamine oxidase B | Xp11.4-p11.3 | 0.865 | 0.808 | 7 |
| 41859_at | UST | Uronyl-2-sulfotransferase | 6q24.3-q25.1 | 0.865 | 0.877 | 1 |
| 38220_at | DPYD | Dihydropyrimidine | 1p22 | 0.844 | 0.820 | 5 |
| | | dehydrogenase | | | | |
| 37015_at | ALDH1A1 | Aldehyde dehydrogenase | 9q21 | 0.820 | 0.751 | |
| | | 1 family, member A1 | | | | |
| 1290_g_at | GSTM5 | Glutathione S-transferase | 1p13.3 | 0.814 | 0.776 | 19 |
| | | M5 | | | | |
| 37599_at | AOX1 | Aldehyde oxidase 1 | 2q33 | 0.743 | 0.652 | |
| 32805_at | AKR1C1 | Aldo-keto reductase | 10p15-p14 | 0.736 | 0.586 | |
| | | family 1, member C1 | | | | |
| 34169_s_at | OCRL | Oculocerebrorenal | Xq25-q26.1 | 0.722 | 0.661 | |
| | | syndrome of Lowe | | | | |
| 32618_at | BLVRA | Biliverdin reductase A | 7p14-cen | 0.430 | 0.786 | 11* |

Cytoskeleton

| | | | | | | |
|---|---|---|---|---|---|---|
| 32145_at | ADD1 | Adducin 1 (alpha) | 4p16.3 | 0.817 | 0.786 | 12 |
| 40488_at | DMD | Dystrophin (muscular | Xp21.2 | 0.778 | 0.698 | |
| | | dystrophy, Duchenne and | | | | |
| | | Becker types) | | | | |
| 38669_at | SLK | Ste20-related serine/ | 10q25.1 | 0.770 | 0.712 | |
| | | threonine kinase | | | | |
| 41738_at | CALD1 | Caldesmon 1 | 7q33 | 0.755 | 0.707 | |
| 34772_at | CORO2B | Coronin, actin binding | 15q22.2-q22.31 | 0.729 | 0.748 | |
| | | protein, 2B | | | | |

Extracellular matrix

| | | | | | | |
|---|---|---|---|---|---|---|
| 39673_i_at | ECM3 | Extracellular matrix | 9q22.3 | 0.827 | 0.698 | |
| | | protein 3 | | | | |
| 39674_r_at | ECM2 | Extracellular matrix | 9q22.3 | 0.811 | 0.699 | |
| | | protein 2 | | | | |
| 36917_at | LAMα2 | Laminin, alpha 2 | 6q22-q23 | 0.810 | 0.752 | |
| 41449_at | SGCE | Sarcoglycan, epsilon | 7q21-q22 | 0.778 | 0.762 | 28 |
| 36627_at | SPARCL1 | SPARC-like 1 (mast9, | 4q21.3 | 0.770 | 0.675 | |
| | | hevin) | | | | |
| 32535_at | FBN1 | Fibrillin 1(Marfan | 15q21.1 | 0.729 | 0.582 | |
| | | syndrome) | | | | |

Others

| | | | | | | |
|---|---|---|---|---|---|---|
| 35717_at | ABCA8 | ATP-binding cassette, | 17q24 | 0.847 | 0.783 | 16 |
| | | subfamily A (ABC1), | | | | |
| | | member 8 | | | | |
| 37394_at | C7 | Complement component 7 | 5p13 | 0.791 | 0.656 | |
| 40767_at | TFPI | Tissue factor pathway | 2q31-q32.1 | 0.779 | 0.640 | |
| | | inhibitor | | | | |
| 41137_at | PPP1R12B | Protein phosphatase 1, | 1q32.1 | 0.768 | 0.631 | |
| | | regulatory (inhibitor) | | | | |
| | | subunit 12B | | | | |
| 38122_at | SLC23A1 | Solute carrier family 23 | 20p13 | 0.761 | 0.707 | |
| | | (nucleobase transporters), | | | | |
| | | member 1 | | | | |
| 32526_at | JAM3 | Junctional adhesion | 11q25 | 0.749 | 0.631 | |
| | | molecule 3 | | | | |
| 38119_at | GYPC | Glycophorin C (Gerbich | 2q14-q21 | 0.747 | 0.669 | |
| | | blood group) | | | | |
| 38634_at | RBP1 | Retinol binding protein 1, | 3q23 | 0.745 | 0.677 | |
| | | cellular | | | | |
| 32109_at | FXYD1 | FXYD domain containing | 19q13.1 | 0.744 | 0.670 | |
| | | ion transport regulator 1 | | | | |
| 40496_at | C1S | Complement component | 12p13 | 0.734 | 0.617 | |
| | | 1, s subcomponent | | | | |
| 41138_at | MIC2 | Antigen identified by | Xp22.32; | 0.731 | 0.565 | |
| | | monoclonal antibodies | Yp11.3 | | | |
| | | 12E7, F21 and O13 | | | | |
| 40786_at | PPP2R5C | Protein phosphatase 2, | 3p21 | 0.725 | 0.705 | |
| | | regulatory subunit B (B56), | | | | |
| | | gamma isoform | | | | |
| 35354_at | RPL3 | Ribosomal protein L3 | 22q13 | 0.723 | 0.667 | |
| 36873_at | VLDLR | Very low density | 9p24 | 0.722 | 0.633 | |
| | | lipoprotein receptor | | | | |

Unknown

| | | | | | | |
|---|---|---|---|---|---|---|
| 40423_at | KIAA0903 | KIAA0903 protein | 2p13.3 | 0.844 | 0.785 | 14 |
| 35742_at | LKAP | Limkain b1 | 16p13.2 | 0.815 | 0.708 | |
| 39750_at | Unknown | — | — | 0.796 | 0.718 | |
| 35645_at | Unknown | — | — | 0.795 | 0.714 | |
| 38717_at | DKFZP586A0522 | DKFZP586A0522 protein | 12q11 | 0.793 | 0.789 | 9 |
| 36867_at | Unknown | LOC92710 | 1q31.1 | 0.785 | 0.739 | |
| 39852_at | TAHCCP1 | Transactivated by hepatitis | 13q13.1 | 0.783 | 0.738 | |
| | | C virus core protein 1 | | | | |
| 40063_at | NDP52 | Nuclear domain 10 protein | 17q23.2 | 0.777 | 0.729 | |
| 41685_at | KIAA0752 | KIAA0752 protein | 5q35.3 | 0.777 | 0.871 | 2 |
| 37446_at | KIAA0443 | | Xq22.1 | 0.771 | 0.613 | |
| 36894_at | Unknown | — | 22q12.3-13.1 | 0.768 | 0.634 | |
| 41273_at | Unknown | — | — | 0.766 | 0.758 | |
| 40861_at | MRGX | MORF-related gene X | Xq22 | 0.760 | 0.689 | |
| 35164_at | WFS1 | Wolfram syndrome 1 | 4p16 | 0.758 | 0.621 | |
| | | (wolframin) | | | | |
| 39400_at | KIAA1055 | | 15q24.1 | 0.758 | 0.634 | |
| 38113_at | SYNE-1 | Synaptic nuclei expressed | 6q25 | 0.754 | 0.681 | |
| | | gene 1 | | | | |
| 34760_at | KIAA0022 | KIAA0022 gene product | 2q24.2 | 0.749 | 0.699 | |
| 33690_at | Unknown | — | — | 0.747 | 0.565 | |
| 41478_at | KIAA1043 | | 22q12.1 | 0.745 | 0.653 | |
| 32076_at | DSCR1L1 | Down syndrome critical | 6p12.3 | 0.741 | 0.713 | |
| | | region gene 1-like 1 | | | | |
| 40853_at | ATP10D | ATPase, Class V, type 10D | 4p12 | 0.740 | 0.647 | |
| 39714_at | SH3BGRL | SH3 domain binding | Xq13.3 | 0.738 | 0.743 | |
| | | glutamic acid-rich protein | | | | |
| | | like | | | | |
| 36577_at | MIG2 | Mitogen inducible 2 | 14q22.1 | 0.736 | 0.549 | |
| 38643_at | Unknown | — | — | 0.735 | 0.714 | |
| 38968_at | SH3BP5 | SH3-domain binding | 3p24.3 | 0.729 | 0.683 | |
| | | protein 5 (BTK-associated) | | | | |
| 37743_at | FEZ1 | Fasciculation and | 11q24.2 | 0.729 | 0.617 | |
| | | elongation protein zeta 1 | | | | |
| | | (zygin I) | | | | |
| 32251_at | FLJ21174 | Hypothetical protein | Xq22.1 | 0.728 | 0.615 | |
| | | FLJ21174 | | | | |
| 39743_at | FLJ20580 | | 1p33 | 0.702 | 0.775 | 20* |
| 36396_at | Unknown | — | — | 0.688 | 0.785 | 15* |
| 35173_at | DXS1283E | | Xp22.3 | 0.617 | 0.774 | 22* |
| 38394_at | KIAA0089 | | 3p22.2 | 0.500 | 0.790 | 8* |
| 40916_at | FLJ10097 | | Xq22.1-q22.3 | 0.359 | 0.777 | 18* |

Note:
Gene symbols in bold indicated genes detected with 2 separate probe sets. Absolute CC values are shown for expression levels analyzed in all 36 samples (R1) and in the 18 test samples only (R2). In each functional category, probe sets are listed by descending R1 values.
*Indicates genes from the 28 classification set not ranked within the 100 highest R1 values.

TABLE 9
Full Set of 900 Genes Differentially Affected in Ovarian Cancer

| | | | | Cytogenetic |
|---|---|---|---|---|
| | Probe Sets | CC | Gene Symbol | Location |
| 1 | 41859_at | 0.877 | UST | 6q24.3-q25.1 |
| 2 | 41685_at | 0.871 | KIAA0752 | 5q35.3 |
| 3 | 36160_s_at | 0.846 | PTPRN2 | 7q36 |
| 4 | 755_at | 0.822 | ITPR1 | 3p26-p25 |
| 5 | 38220_at | 0.82 | DPYD | 1p22 |
| 6 | 32668_at | 0.809 | SSBP2 | 5q14.1 |
| 7 | 37628_at | 0.808 | MAOB | Xp11.4-p11.3 |
| 8 | 38394_at | 0.79 | KIAA0089 | 3p22.2 |
| 9 | 38717_at | 0.789 | DKFZP586A0522 | 12q11 |
| 10 | 1761_at | 0.789 | PDGFRL | 8p22-p21.3 |
| 11 | 32618_at | 0.786 | BLVRA | 7p14-cen |
| 12 | 32145_at | 0.786 | ADD1 | 7p16.3 |
| 13 | 34257_at | 0.786 | AIP1 | 7q21 |
| 14 | 40423_at | 0.785 | KIAA0903 | 2p13.3 |
| 15 | 36396_at | 0.785 | cDNA DKFZp586N | 4p16.3 |
| 16 | 35717_at | 0.783 | ABCA8 | 17q24 |
| 17 | 41796_at | 0.779 | PLCL2 | 3p24.3 |
| 18 | 40916_at | 0.777 | Gene for hypothetical | |
| | | | protein FLJ10097 | |
| 19 | 1290_g_at | 0.776 | GSTM5 | 1p13.3 |
| 20 | 39743_at | 0.775 | FLJ20580 | 1p33 |
| 21 | 36073_at | 0.774 | NDN | 15q11.2-q12 |
| 22 | 35173_at | 0.774 | DXS1283E | Xp22.3 |
| 23 | 1897_at | 0.77 | TGFBR3 | 1p33-p32 |
| 24 | 872_i_at | 0.77 | IRS1 | 2q36 |
| 25 | 38120_at | 0.768 | PKD2 | 4q21-q23 |
| 26 | 1577_at | 0.767 | AR | Xq11.2-q12 |
| 27 | 36948_at | 0.766 | CRI1 | 15q21.1-q21.2 |
| 28 | 41449_at | 0.762 | SGCE | 7q21-q22 |
| 29 | 40480_s_at | 0.761 | FYN | 6q21 |
| 30 | 34842_at | 0.759 | SNRPN | 15q12 |
| 31 | 41273_at | 0.758 | EST | |
| 32 | 1396_at | 0.756 | IGFBP5 | 2q33-q36 |
| 33 | 38047_at | 0.756 | RBPMS | 8p12-p11 |
| 34 | 35738_at | 0.755 | HMGN4 | 6p21.3 |
| 35 | 40876_at | 0.754 | GYG | 3q24-q25.1 |
| 36 | 35783_at | 0.753 | VAMP3 | 1p36.23 |
| 37 | 37242_at | 0.753 | MGC5149 | 16q12.2 |
| 38 | 36917_at | 0.752 | LAMA2 | 6q22-q23 |
| 39 | 37015_at | 0.751 | ALDH1A1 | 9q21.13 |
| 40 | 32664_at | 0.749 | RNASE4 | 14q11.1 |
| 41 | 34772_at | 0.748 | CORO2B | 15q22.2-q22.31 |
| 42 | 38650_at | 0.748 | IGFBP5 | 2q33-q36 |
| 43 | 39025_at | 0.746 | TOM7 | 7p21.3 |
| 44 | 40961_at | 0.745 | SMARCA2 | 9p22.3 |
| 45 | 32777_at | 0.743 | WRB | 21q22.3 |
| 46 | 39714_at | 0.743 | SH3BGRL | Xq13.3 |
| 47 | 35316_at | 0.743 | RAGA | 9p21.2 |
| 48 | 38318_at | 0.741 | FAM8A1 | 6p22-p23 |
| 49 | 38802_at | 0.739 | PGRMC1 | Xq22-q24 |
| 50 | 36867_at | 0.739 | cDNA FLJ34019 fis | |
| 51 | 39852_at | 0.738 | TAHCCP1 | 13q13.1 |
| 52 | 35435_s_at | 0.736 | HADHSC | 4q22-q26 |
| 53 | 38439_at | 0.736 | NFE2L1 | 17q21.3 |
| 54 | 1909_at | 0.736 | BCL2 | 18q21.3 |
| 55 | 33942_s_at | 0.735 | STXBP1 | 9q34.1 |
| 56 | 1640_at | 0.734 | ST13 | 22q13.2 |
| 57 | 227_g_at | 0.733 | PRKAR1A | 17q23-q24 |
| 58 | 2010_at | 0.732 | SKP1A | 5q31 |
| 59 | 40063_at | 0.729 | NDP52 | 17q23.2 |
| 60 | 38176_at | 0.729 | GNB5 | 15q15.3 |
| 61 | 39350_at | 0.727 | GPC3 | Xq26.1 |
| 62 | 39037_at | 0.726 | MLLT2 | 4q21 |
| 63 | 851_s_at | 0.725 | IRS1 | 2q36 |
| 64 | 39556_at | 0.725 | SPTBN1 | 2p21 |
| 65 | 2039_s_at | 0.723 | FYN | 6q21 |
| 66 | 41744_at | 0.723 | OPTN | 10p12.33 |
| 67 | 32695_at | 0.722 | HTATSF1 | Xq26.1-q27.2 |
| 68 | 36915_at | 0.72 | CTSO | 4q31-q32 |
| 69 | 38982_at | 0.719 | TERF2IP | 16q22.3 |
| 70 | 1348_s_at | 0.718 | PCCA | 13q32 |
| 71 | 39750_at | 0.718 | EST | |
| 72 | 37643_at | 0.716 | TNFRSF6 | 10q24.1 |
| 73 | 39376_at | 0.715 | Nbak2 | 1p11.2 |
| 74 | 38695_at | 0.715 | NDUFS4 | 5q11.1 |
| 75 | 35645_at | 0.714 | cDNA DKFZp586G1520 | |
| 76 | 38643_at | 0.714 | LOC92689 | 4p15.1 |
| 77 | 38375_at | 0.713 | ESD | 13q14.1-q14.2 |
| 78 | 32076_at | 0.713 | DSCR1L1 | 6p12.3 |
| 79 | 38669_at | 0.712 | SLK | 10q25.1 |
| 80 | 37373_at | 0.71 | UGP2 | 2p14-p13 |
| 81 | 37532_at | 0.71 | ACADM | 1p31 |
| 82 | 39165_at | 0.71 | NIFU | 12q24.1 |
| 83 | 34163_g_at | 0.709 | RBPMS | 8p12-p11 |
| 84 | 35742_at | 0.708 | LKAP | 16p13.13 |
| 85 | 40607_at | 0.708 | DPYSL2 | 8p22-p21 |
| 86 | 41738_at | 0.707 | CALD1 | 7q33 |
| 87 | 38122_at | 0.707 | SLC23A1 | 20p13 |
| 88 | 32747_at | 0.706 | ALDH2 | 12q24.2 |
| 89 | 40786_at | 0.705 | PPP2R5C | 3p21 |
| 90 | 33198_at | 0.705 | BART1 | 16q13 |
| 91 | 38745_at | 0.702 | LIPA | 10q23.2-q23.3 |
| 92 | 39243_s_at | 0.702 | PSIP2 | 9p22.1 |
| 93 | 33936_at | 0.702 | GALC | 14q31 |
| 94 | 39397_at | 0.7 | NR2F2 | 15q26 |
| 95 | 41147_at | 0.699 | MGC4276 | 9q22.1 |
| 96 | 34760_at | 0.699 | KIAA0022 | 2q24.2 |
| 97 | 39674_r_at | 0.699 | ECM2 | 9q22.3 |
| 98 | 39401_at | 0.699 | IMAGE clone 3460701 | |
| 99 | 40488_at | 0.698 | DMD | Xp21.2 |
| 100 | 39673_i_at | 0.698 | ECM2 | 9q22.3 |
| 101 | 39864_at | 0.698 | CIRBP | 19p13.3 |
| 102 | 1127_at | 0.697 | RPS6KA1 | 3 |
| 103 | 40674_s_at | 0.697 | HOXC6 | 12q13.3 |
| 104 | 36975_at | 0.696 | MGC8721 | 8p12 |
| 105 | 41049_at | 0.694 | IRS1 | 2q36 |
| 106 | 32593_at | 0.692 | KIAA0084 | 3p24.3 |
| 107 | 1629_s_at | 0.692 | PTPN13 | 4q21.3 |
| 108 | 36620_at | 0.692 | SOD1 | 21q22.11 |
| 109 | 32764_at | 0.691 | PGCP | 8q22.2 |
| 110 | 35785_at | 0.691 | GABARAPL1 | 12p13.1 |
| 111 | 39681_at | 0.69 | ZNF145 | 11q23.1 |
| 112 | 39438_at | 0.69 | CREBL2 | 12p13 |
| 113 | 32778_at | 0.689 | ITPR1 | 3p26-p25 |
| 114 | 40861_at | 0.689 | MRGX | Xq22 |
| 115 | 34990_at | 0.688 | SETBP1 | 18q21.1 |
| 116 | 1736_at | 0.688 | IGFBP6 | 12q13 |
| 117 | 41771_g_at | 0.687 | MAOA | Xp11.4-p11.3 |
| 118 | 31852_at | 0.687 | DKFZP564O043 | 7p21 |
| 119 | 36542_at | 0.686 | SLC9A6 | Xq26.3 |
| 120 | 37379_at | 0.684 | SF3A3 | 1p35.2 |
| 121 | 38968_at | 0.683 | SH3BP5 | 3p24.3 |
| 122 | 39691_at | 0.683 | SH3GLB1 | 1p22 |
| 123 | 38211_at | 0.681 | ZNF288 | 3q13.2 |
| 124 | 38113_at | 0.681 | SYNE-1 | 6q25 |
| 125 | 40601_at | 0.68 | BBP | 1p32.1 |
| 126 | 2092_s_at | 0.679 | SPP1 | 4q21-q25 |
| 127 | 33830_at | 0.679 | HSOBRGRP | 1 |
| 128 | 34637_f_at | 0.678 | ADH1A | 4q21-q23 |
| 129 | 38634_at | 0.677 | RBP1 | 3q23 |
| 130 | 35643_at | 0.677 | NUCB2 | 11p15.1-p14 |
| 131 | 41000_at | 0.677 | CHES1 | 14q24.3-q31 |
| 132 | 37828_at | 0.677 | FLJ11220 | 1p11.2 |
| 133 | 35163_at | 0.676 | KIAA1041 | 1pter-q31.3 |
| 134 | 36627_at | 0.675 | SPARCL1 | 4q21.3 |
| 135 | 853_at | 0.674 | NFE2L2 | 2q31 |
| 136 | 34356_at | 0.673 | SURB7 | 12p12.3 |
| 137 | 38013_at | 0.672 | ATIP1 | 8p22 |
| 138 | 38664_at | 0.672 | CFDP1 | 16q22.2-q22.3 |
| 139 | 32087_at | 0.672 | HSF2 | 6q22.33 |
| 140 | 38768_at | 0.671 | HADHSC | 4q22-q26 |
| 141 | 34877_at | 0.671 | JAK1 | 1p32.3-p31.3 |
| 142 | 1090_f_at | 0.671 | ? | |
| 143 | 32109_at | 0.67 | FXYD1 | 19q13.1 |
| 144 | 34859_at | 0.669 | MAGED2 | Xp11.4-p11.1 |
| 145 | 38119_at | 0.669 | GYPC | 2q14-q21 |
| 146 | 31510_s_at | 0.668 | H3F3B | 17q25 |
| 147 | 1058_at | 0.668 | WASF3 | 13q12 |
| 148 | 40570_at | 0.668 | FOXO1A | 13q14.1 |
| 149 | 39091_at | 0.668 | JWA | 3p14 |
| 150 | 32057_at | 0.667 | P37NB | 7q11.22 |
| 151 | 31993_f_at | 0.667 | EST | |
| 152 | 35354_at | 0.667 | SYNGR1 | 22q13.1 |
| 153 | 40775_at | 0.666 | ITM2A | Xq13.3-Xq21.2 |
| 154 | 40140_at | 0.666 | ZFP103 | 2p11.2 |
| 155 | 37406_at | 0.665 | MAPRE2 | 18q12.1 |
| 156 | 38685_at | 0.665 | STX12 | 1p35-34.1 |
| 157 | 34363_at | 0.665 | SEPP1 | 5q31 |
| 158 | 33351_at | 0.664 | GC20 | 3p21.33 |
| 159 | 41655_at | 0.664 | MID2 | Xq22 |
| 160 | 39072_at | 0.662 | MXI1 | 10q24-q25 |
| 161 | 36544_at | 0.662 | clone | IMAGE: 3610040 |
| 162 | 32542_at | 0.662 | FHL1 | Xq26 |
| 163 | 35767_at | 0.662 | GABARAPL2 | 16q22.3-q24.1 |
| 164 | 34355_at | 0.662 | MECP2 | Xq28 |
| 165 | 1578_g_at | 0.661 | AR | Xq11.2-q12 |
| 166 | 41656_at | 0.661 | NMT2 | 10p12.33-p12.32 |
| 167 | 34169_s_at | 0.661 | OCRL | Xq25-q26.1 |
| 168 | 41662_at | 0.661 | DKFZP566B183 | 12p13.32 |
| 169 | 40203_at | 0.66 | SUI1 | 17 |
| 170 | 39315_at | 0.659 | ANGPT1 | 8q22.3-q23 |
| 171 | 226_at | 0.657 | PRKAR1A | 17q23-q24 |
| 172 | 33878_at | 0.657 | FLJ13612 | 2q36.1 |
| 173 | 38408_at | 0.656 | TM4SF2 | Xq11.4 |
| 174 | 35704_at | 0.656 | HRASLS3 | 11q13.1 |
| 175 | 37394_at | 0.656 | C7 | 5p13 |
| 176 | 39829_at | 0.656 | ARL7 | 2q37.2 |
| 177 | 40770_f_at | 0.655 | HNRPDL | 4q13-q21 |
| 178 | 35846_at | 0.655 | THRA | 17q11.2 |
| 179 | 176_at | 0.654 | PPP2R5C | 3p21 |
| 180 | 36690_at | 0.654 | NR3C1 | 5q31 |
| 181 | 39351_at | 0.653 | CD59 | 11p13 |
| 182 | 41478_at | 0.653 | KIAA1043 | 22q12.1 |
| 183 | 950_at | 0.653 | TLOC1 | 3q26.2-q27 |
| 184 | 35359_at | 0.653 | PUM2 | 2p22-p21 |
| 185 | 1135_at | 0.652 | GPRK5 | 10q24-qter |
| 186 | 36091_at | 0.652 | SCAP2 | 7p21-p15 |
| 187 | 2003_s_at | 0.652 | MSH6 | 2p16 |
| 188 | 35246_at | 0.652 | TYRO3 | 15q15.1-q21.1 |
| 189 | 40576_f_at | 0.652 | HNRPDL | 4q13-q21 |
| 190 | 37599_at | 0.652 | AOX1 | 2q33 |
| 191 | 35209_at | 0.652 | KIAA0766 | 3p22.1 |
| 192 | 38916_at | 0.651 | CXorf6 | Xq28 |
| 193 | 33126_at | 0.651 | AD-017 | 3p21.31 |
| 194 | 37706_at | 0.65 | GLG1 | 16q22-q23 |
| 195 | 40077_at | 0.65 | ACO1 | 9p22-p13 |
| 196 | 37294_at | 0.649 | BTG1 | 12q22 |
| 197 | 32597_at | 0.648 | RBL2 | 16q12.2 |
| 198 | 32768_at | 0.648 | FLJ21007 | 13q21.1 |
| 199 | 36543_at | 0.648 | F3 | 1p22-p21 |
| 200 | 37736_at | 0.647 | PCMT1 | 6q24-q25 |
| 201 | 40853_at | 0.647 | ATP10D | 4p12 |
| 202 | 41830_at | 0.647 | KIAA0494 | 1pter-p22.1 |
| 203 | 37197_s_at | 0.647 | DKFZP564A033 | 2 |
| 204 | 37205_at | 0.645 | FBXL7 | 5p15.1 |
| 205 | 40617_at | 0.645 | SAH | 16p13.11 |
| 206 | 35681_r_at | 0.644 | ZFHX1B | 2q22 |
| 207 | 41594_at | 0.644 | JAK1 | 1p32.3-p31.3 |
| 208 | 192_at | 0.643 | TAF7 | 5q31 |
| 209 | 654_at | 0.643 | MXI1 | 10q24-q25 |
| 210 | 41742_s_at | 0.642 | OPTN | 10p12.33 |
| 211 | 32676_at | 0.642 | ALDH6A1 | 14q24.3 |
| 212 | 35752_s_at | 0.642 | PROS1 | 3p11-q11.2 |
| 213 | 34774_at | 0.642 | PPT1 | 1p32 |
| 214 | 38892_at | 0.642 | KIAA0240 | 6p21.1 |
| 215 | 659_g_at | 0.641 | THBS2 | 6q27 |
| 216 | 41288_at | 0.641 | CALM1 | 14q24-q31 |
| 217 | 35955_at | 0.64 | ? | |
| 218 | 924_s_at | 0.64 | PPP2CB | 8p12-p11.2 |
| 219 | 39360_at | 0.64 | SNX3 | 6q22.1 |
| 220 | 40767_at | 0.64 | TFPI | 2q31-q32.1 |
| 221 | 237_s_at | 0.639 | PPP2CA | 5q23-q31 |
| 222 | 35782_at | 0.638 | KIAA0657 | 2q36.3 |
| 223 | 1678_g_at | 0.638 | IGFBP5 | 2q33-q36 |
| 224 | 41505_r_at | 0.638 | MAF | 16q22-q23 |
| 225 | 39715_at | 0.638 | cDNA FLJ31079 fis | |
| 226 | 34198_at | 0.638 | PTPN13 | 4q21.3 |
| 227 | 34821_at | 0.638 | DKFZP586D0623 | 6q23.1-q24.1 |
| 228 | 33868_at | 0.637 | dJ222E13.2 | 22q13.2 |
| 229 | 2086_s_at | 0.637 | TYRO3 | 15q15.1-q21.1 |
| 230 | 1147_at | 0.637 | NR2F1 | 5q14 |
| 231 | 40145_at | 0.636 | TOP2A | 17q21-q22 |
| 232 | 40211_at | 0.636 | HNRPA1 | 12q13.1 |
| 233 | 37617_at | 0.636 | KIAA1128 | 10q23.31 |
| 234 | 36489_at | 0.636 | PRPS1 | Xq21-q27 |
| 235 | 36488_at | 0.636 | EGFL5 | 9q32-q33.3 |
| 236 | 33443_at | 0.635 | TDE1L | 6q22.32 |
| 237 | 39369_at | 0.635 | KIAA0935 | 4p16.1 |
| 238 | 36894_at | 0.634 | CBX7 | 22q13.1 |
| 239 | 35234_at | 0.634 | RECK | 9p13-p12 |
| 240 | 39400_at | 0.634 | KIAA1055 | 15q24.1 |
| 241 | 38074_at | 0.634 | AP3S1 | 5q22 |
| 242 | 34803_at | 0.633 | USP12 | 5q33-q34 |
| 243 | 39441_at | 0.633 | LANCL1 | 2q33-q35 |
| 244 | 36873_at | 0.633 | VLDLR | 9p24 |
| 245 | 38985_at | 0.633 | LEPROTL1 | 8p21.2-p21.1 |
| 246 | 33911_at | 0.633 | cDNA DKFZp564P116 | |
| 247 | 41638_at | 0.633 | KIAA0073 | 5q12.3 |
| 248 | 41277_at | 0.632 | SAP18 | 13q11 |
| 249 | 38342_at | 0.632 | KIAA0239 | 5q31.1 |
| 250 | 35754_at | 0.632 | TMP21 | 14q24.3 |
| 251 | 32526_at | 0.631 | JAM3 | 11q25 |
| 252 | 41137_at | 0.631 | PPP1R12B | 1q32.1 |
| 253 | 33857_at | 0.63 | p47 | 20p13 |
| 254 | 871_s_at | 0.63 | HLF | 17q22 |
| 255 | 40399_r_at | 0.63 | MEOX2 | 7p22.1-p21.3 |
| 256 | 39428_at | 0.63 | LNK | 12q24 |
| 257 | 31872_at | 0.63 | SS18 | 18q11.2 |
| 258 | 41634_at | 0.629 | KIAA0256 | 15q15.1 |
| 259 | 39731_at | 0.629 | RBMX | Xq26 |
| 260 | 33136_at | 0.628 | ? | |
| 261 | 38353_at | 0.628 | TUBGCP3 | 13q34 |
| 262 | 32708_g_at | 0.627 | KATNA1 | 6q25.1 |
| 263 | 40994_at | 0.627 | GPRK5 | 10q24-qter |
| 264 | 33222_at | 0.626 | FZD7 | 2q33 |
| 265 | 1327_s_at | 0.626 | MAP3K5 | 6q22.33 |
| 266 | 40039_g_at | 0.625 | ST7 | 7q31.1-q31.3 |
| 267 | 35228_at | 0.624 | CPT1B | 22q13.33 |
| 268 | 38693_at | 0.623 | ATP5L | 3q27 |
| 269 | 39431_at | 0.622 | NPEPPS | 17q21 |
| 270 | 35784_at | 0.622 | VAMP3 | 1p36.23 |
| 271 | 538_at | 0.622 | CD34 | 1q32 |
| 272 | 36119_at | 0.622 | CAV1 | 7q31.1 |
| 273 | 218_at | 0.621 | IK | 5q31.3 |
| 274 | 35164_at | 0.621 | WFS1 | 4p16 |
| 275 | 39856_at | 0.62 | RPL36AL | 14q21 |
| 276 | 41529_g_at | 0.62 | cDNA DKFZp434M162 | |
| 277 | 37715_at | 0.619 | SNW1 | 14q24.3 |
| 278 | 33302_at | 0.619 | SSPN | 12p11.2 |
| 279 | 1731_at | 0.619 | PDGFRA | 4q11-q13 |
| 280 | 35741_at | 0.619 | PIP5K2B | 17q12 |
| 281 | 35356_at | 0.619 | MGC9651 | 4p16.1 |
| 282 | 39582_at | 0.618 | cDNA DKFZp586D1122 | |
| 283 | 911_s_at | 0.618 | CALM2 | 2p21 |
| 284 | 41620_at | 0.618 | KIAA0716 | 7q21.13 |
| 285 | 33249_at | 0.618 | NR3C2 | 4q31.1 |
| 286 | 40841_at | 0.618 | TACC1 | 8p11 |
| 287 | 37595_at | 0.618 | cDNA DKFZp547E184 | |
| 288 | 37958_at | 0.617 | BCMP1 | Xp11.4 |
| 289 | 37748_at | 0.617 | KIAA0232 | 4p16.1 |
| 290 | 40496_at | 0.617 | C1S | 12p13 |
| 291 | 37743_at | 0.617 | FEZ1 | 11q24.2 |
| 292 | 35335_at | 0.616 | ROCK2 | 2p24 |
| 293 | 33862_at | 0.615 | PPAP2B | 1pter-p22.1 |
| 294 | 32251_at | 0.615 | FLJ21174 | Xq22.1 |
| 295 | 37486_f_at | 0.615 | MEIS3 | 17p11.2 |
| 296 | 38101_at | 0.615 | BDG-29 | 16q24.2 |
| 297 | 40213_at | 0.615 | SMARCA1 | Xq25 |
| 298 | 32851_at | 0.614 | CUGBP2 | 10p13 |
| 299 | 1211_s_at | 0.614 | CRADD | 12q21.33-q23.1 |
| 300 | 34819_at | 0.614 | CD164 | 6q21 |
| 301 | 34808_at | 0.614 | KIAA0999 | 11q23.3 |
| 302 | 37446_at | 0.613 | KIAA0443 | Xq22.1 |
| 303 | 32792_at | 0.612 | P29 | 1p36.13-p35.1 |
| 304 | 36650_at | 0.612 | CCND2 | 12p13 |
| 305 | 38438_at | 0.612 | NFKB1 | 4q24 |
| 306 | 39701_at | 0.611 | PEG3 | 19q13.4 |
| 307 | 34215_at | 0.611 | DXYS155E | Xp22.32 |
| 308 | 32259_at | 0.611 | EZH1 | 17q21.1-q21.3 |
| 309 | 40839_at | 0.611 | UBL3 | 13q12-q13 |
| 310 | 39055_at | 0.61 | SRI | 7q21.1 |
| 311 | 40508_at | 0.61 | GSTA4 | 6p12.1 |
| 312 | 37985_at | 0.608 | LMNB1 | 5q23.3-q31.1 |
| 313 | 33799_at | 0.607 | SIAH2 | 3q25 |
| 314 | 37638_at | 0.607 | DOCK1 | 10q26.13-q26.3 |
| 315 | 33140_at | 0.607 | B3GNT6 | 11q12.1 |
| 316 | 40202_at | 0.606 | BTEB1 | 9q13 |
| 317 | 39033_at | 0.606 | C1orf8 | 1p36-p31 |
| 318 | 34789_at | 0.606 | SERPINB6 | 6p25 |
| 319 | 33817_at | 0.606 | HNRPA3 | 10q11.1 |
| 320 | 1719_at | 0.606 | MSH3 | 5q11-q12 |
| 321 | 38923_at | 0.605 | FRG1 | 4q35 |
| 322 | 41338_at | 0.605 | AES | 19p13.3 |
| 323 | 35751_at | 0.605 | SDHB | 1p36.1-p35 |
| 324 | 1377_at | 0.605 | NFKB1 | 4q24 |
| 325 | 33123_at | 0.605 | HRIHFB2206 | 16q22.1 |
| 326 | 933_f_at | 0.604 | ZNF91 | 19p13.1-p12 |
| 327 | 32696_at | 0.604 | PBX3 | 9q33-q34 |
| 328 | 1323_at | 0.604 | UBB | 17p12-p11.2 |
| 329 | 34349_at | 0.603 | SEC63L | 6q21 |
| 330 | 37731_at | 0.602 | EPS15 | 1p32 |
| 331 | 37315_f_at | 0.602 | C14orf11 | 14q12 |
| 332 | 36695_at | 0.602 | cDNA FLJ40364 fis | |
| 333 | 31867_at | 0.602 | ? | 3q13.12 |
| 334 | 31944_at | 0.601 | TULP3 | 12p13.3 |
| 335 | 1070_at | 0.601 | GTF2B | 1p22-p21 |
| 336 | 38254_at | 0.601 | KIAA0882 | 4q31.1 |
| 337 | 37710_at | 0.6 | MEF2C | 5q14 |
| 338 | 33343_at | 0.6 | RNF14 | 5q23.3-q31.1 |
| 339 | 32779_s_at | 0.599 | ITPR1 | 3p26-p25 |
| 340 | 33865_at | 0.599 | BS69 | 10p14 |
| 341 | 31508_at | 0.599 | TXNIP | 1q12 |
| 342 | 40419_at | 0.599 | STOM | 9q34.1 |
| 343 | 33915_at | 0.599 | FLJ23027 | 14q32.32 |
| 344 | 38364_at | 0.599 | BCE-1 | 9q21.32 |
| 345 | 38050_at | 0.599 | BTF | 6q22-q23 |
| 346 | 38046_at | 0.599 | IK | 5q31.3 |
| 347 | 38820_at | 0.598 | 15-Sep | 1p31 |
| 348 | 32713_at | 0.598 | GOLGA1 | 9q34.11 |
| 349 | 32107_at | 0.598 | C21orf25 | 21q22.3 |
| 350 | 38727_at | 0.597 | SDNSF | 2p21 |
| 351 | 38839_at | 0.597 | PFN2 | 3q25.1-q25.2 |
| 352 | 38033_at | 0.597 | DKFZP564M1416 | 8q11.22 |
| 353 | 729_i_at | 0.597 | MUC3A | 7q22 |
| 354 | 1507_s_at | 0.596 | EDNRA | 4q31.21 |
| 355 | 33103_s_at | 0.596 | ADD3 | 10q24.2-q24.3 |
| 356 | 39436_at | 0.596 | BNIP3L | 8p21 |
| 357 | 39097_at | 0.595 | SON | 21q22.11 |
| 358 | 39294_at | 0.595 | NR2F1 | 5q14 |
| 359 | 41333_at | 0.595 | CENTB2 | 3q29 |
| 360 | 38116_at | 0.595 | KIAA0101 | 15q22.1 |
| 361 | 32780_at | 0.594 | BPAG1 | 6p12-p11 |
| 362 | 41385_at | 0.594 | EPB41L3 | 18p11.32 |
| 363 | 38400_at | 0.594 | DKFZP434D1335 | 19q13.12 |
| 364 | 32841_at | 0.594 | ZNF9 | 3q21 |
| 365 | 41420_at | 0.593 | IGFBP5 | 2q33-q36 |
| 366 | 34860_g_at | 0.593 | MAGED2 | Xp11.4-p11.1 |
| 367 | 38415_at | 0.593 | PTP4A2 | 1p35 |
| 368 | 38317_at | 0.593 | TCEAL1 | Xq22.1 |
| 369 | 39939_at | 0.593 | COL4A6 | Xq22 |
| 370 | 41405_at | 0.592 | SFRP4 | 7p14.1 |
| 371 | 36980_at | 0.592 | PROL2 | 6q16.1 |
| 372 | 39147_g_at | 0.592 | ATRX | Xq13.1-q21.1 |
| 373 | 32700_at | 0.592 | GBP2 | 1p22.1 |
| 374 | 39986_at | 0.591 | DKFZP586D0919 | 12q13.2 |
| 375 | 38690_at | 0.591 | C3orf4 | 3p11-q11 |
| 376 | 1848_at | 0.591 | RAP1A | 1p13.3 |
| 377 | 36636_at | 0.59 | OAT | 10q26 |
| 378 | 37230_at | 0.59 | KIAA0469 | 1p36.23 |
| 379 | 37107_at | 0.59 | PPM1D | 17q23.1 |
| 380 | 33870_at | 0.589 | C5orf7 | 5q31 |
| 381 | 33229_at | 0.589 | RPS6KA3 | Xp22.2-p22.1 |
| 382 | 36159_s_at | 0.589 | PRNP | 2opter-p12 |
| 383 | 32337_at | 0.589 | RPL21 | 10q26.13 |
| 384 | 508_at | 0.589 | SUPT4H1 | 17q21-q23 |
| 385 | 41424_at | 0.588 | PON3 | 7q21.3 |
| 386 | 32667_at | 0.588 | COL4A5 | Xq22 |
| 387 | 39979_at | 0.588 | F10 | 13q34 |
| 388 | 34753_at | 0.587 | SYBL1 | Xq28 |
| 389 | 39082_at | 0.587 | ANXA6 | 5q32-q34 |
| 390 | 39989_at | 0.587 | RAGB | Xp11.21 |
| 391 | 33235_at | 0.587 | NAV3 | |
| 392 | 36825_at | 0.587 | TRIM22 | 11p15 |
| 393 | 41747_s_at | 0.587 | MEF2A | 15q26 |
| 394 | 38279_at | 0.587 | GNAZ | 22q11.22 |
| 395 | 32511_at | 0.586 | cDNA FLJ37094 fis | |
| 396 | 32805_at | 0.586 | AKR1C | 10p15-p14 |
| 397 | 1529_at | 0.586 | 13CDNA73 | 13q12.3 |
| 398 | 34570_at | 0.586 | RPS27A | 2p16 |
| 399 | 31932_f_at | 0.586 | BTF3 | 5q13.1 |
| 400 | 35055_at | 0.586 | BTF3 | 5q13.3 |
| 401 | 32822_at | 0.585 | SLC25A4 | 4q35 |
| 402 | 37131_at | 0.585 | KLK8 | 19q13.3-q13.4 |
| 403 | 35318_at | 0.585 | KIAA0475 | 1p36.13-q41 |
| 404 | 36526_at | 0.584 | EXTL2 | 1p21 |
| 405 | 38837_at | 0.584 | DJ971N18.2 | 20p12 |
| 406 | 36492_at | 0.583 | PSMD9 | 12q24.31-q24.32 |
| 407 | 36515_at | 0.583 | GNE | 9p11.2 |
| 408 | 35737_at | 0.583 | HMGN4 | 6p21.3 |
| 409 | 32535_at | 0.582 | FBN1 | 15q21.1 |
| 410 | 39838_at | 0.582 | CLASP1 | 2q21.3 |
| 411 | 1307_at | 0.582 | XPA | 9q22.3 |
| 412 | 40971_at | 0.581 | KIAA0229 | 6p21.2 |
| 413 | 1319_at | 0.58 | DDR2 | 1q12-q23 |
| 414 | 33892_at | 0.579 | PKP2 | 12p11 |
| 415 | 33800_at | 0.579 | ADCY9 | 16p13.3 |
| 416 | 39790_at | 0.579 | ATP2A2 | 12q23-q24.1 |
| 417 | 37725_at | 0.578 | PPP1CC | 12q24.1-q24.2 |
| 418 | 38711_at | 0.578 | CLASP2 | 3p22.2-p22.1 |
| 419 | 32662_at | 0.578 | KIAA0170 | 6pter-p21.31 |
| 420 | 32582_at | 0.578 | MYH11 | 16p13.13-p13.12 |
| 421 | 41013_at | 0.578 | cDNA DKFZp586M2022 | |
| 422 | 32743_at | 0.577 | KIAA0453 | 1p36.31-p36.11 |
| 423 | 35325_at | 0.577 | RAB14 | 9q32-q34.11 |
| 424 | 36626_at | 0.577 | HSD17B4 | 5q21 |
| 425 | 39038_at | 0.576 | FBLN5 | 14q32.1 |
| 426 | 32160_at | 0.576 | SIAH1 | 16q12 |
| 427 | 1501_at | 0.576 | IGF1 | 12q22-q23 |
| 428 | 32244_at | 0.576 | KIAA0737 | 14q11.1 |
| 429 | 450_g_at | 0.576 | CGR19 | 14q22.1 |
| 430 | 39083_at | 0.575 | UBE2D3 | 4q22.2 |
| 431 | 36596_r_at | 0.575 | GATM | 15q14 |
| 432 | 35276_at | 0.575 | CLDN4 | 7q11.23 |
| 433 | 36578_at | 0.575 | BIRC2 | 11q22 |
| 434 | 32153_s_at | 0.575 | ? | |
| 435 | 33847_s_at | 0.574 | CDKN1B | 12p13.1-p12 |
| 436 | 40432_at | 0.573 | GNS | 12q14 |
| 437 | 39346_at | 0.573 | KHDRBS1 | 1p32 |
| 438 | 38581_at | 0.573 | GNAQ | 9q21 |
| 439 | 37604_at | 0.573 | HNMT | 2q21.3 |
| 440 | 41691_at | 0.572 | KIAA0794 | 3q29 |
| 441 | 201_s_at | 0.571 | B2M | 15q21-q22.2 |
| 442 | 41600_at | 0.571 | PA2G4 | 12q13 |
| 443 | 39762_at | 0.571 | ZNF262 | 1p32-p34 |
| 444 | 32099_at | 0.57 | KIAA0138 | 19p13.3 |
| 445 | 41701_at | 0.57 | C6 | 5p13 |
| 446 | 39150_at | 0.569 | RNF11 | 1pter-p22.1 |
| 447 | 36474_at | 0.569 | KIAA0776 | 6q16.3 |
| 448 | 39685_at | 0.568 | E46L | 22q13.31 |
| 449 | 37391_at | 0.567 | CTSL | 9q21-q22 |
| 450 | 35843_at | 0.567 | NEK9 | 14q24.2 |
| 451 | 41136_s_at | 0.567 | APP | 21q21.3 |
| 452 | 35203_at | 0.566 | MORF | 10q22.2 |
| 453 | 34162_at | 0.566 | RBPMS | 8p12-p11 |
| 454 | 35811_at | 0.566 | RNF13 | 3q25.1 |
| 455 | 39110_at | 0.566 | EIF4B | 12q13.13 |
| 456 | 35331_at | 0.565 | CTNNAL1 | 9q31.2 |
| 457 | 39663_at | 0.565 | MAN2A1 | 5q21-q22 |
| 458 | 41138_at | 0.565 | MIC2 | Xp22.32 |
| 459 | 31936_s_at | 0.565 | LKAP | 16p13.2 |
| 460 | 38470_i_at | 0.565 | APPBP2 | 17q21-q23 |
| 461 | 33690_at | 0.565 | cDNA DKFZp434A202 | |
| 462 | 39846_at | 0.564 | CTSF | 11q13 |
| 463 | 39109_at | 0.564 | C20orf1 | 20q11.2 |
| 464 | 34372_at | 0.564 | UREB1 | Xp11.2 |
| 465 | 32521_at | 0.563 | SFRP1 | 8p12-p11.1 |
| 466 | 35936_g_at | 0.563 | CPT1B | 22q13.33 |
| 467 | 40698_at | 0.563 | CLECSF2 | 12p13-p12 |
| 468 | 324_f_at | 0.562 | BTF3 | 5q13.3 |
| 469 | 333_s_at | 0.562 | RBMS1 | 2q24.2 |
| 470 | 41606_at | 0.562 | DRG1 | 22q12.2 |
| 471 | 40281_at | 0.562 | NEDD5 | 2q37 |
| 472 | 32240_at | 0.562 | PSMD5 | 9q33.3 |
| 473 | 33441_at | 0.561 | TCTA | 3p21 |
| 474 | 39170_at | 0.561 | cDNA DKFZp564J0323 | |
| 475 | 35366_at | 0.56 | NID | 1q43 |
| 476 | 41271_at | 0.56 | SLC7A8 | 14q11.2 |
| 477 | 1530_g_at | 0.56 | 13CDNA73 | 13q12.3 |
| 478 | 33278_at | 0.56 | SAH | 16p13.11 |
| 479 | 38754_at | 0.56 | P8 | 16p11.2 |
| 480 | 37891_at | 0.559 | cDNA DKFZp586F1822 | |
| 481 | 36727_at | 0.558 | ? | |
| 482 | 32506_at | 0.558 | TBC1D1 | 4p14 |
| 483 | 37908_at | 0.557 | GNG11 | 7q31-q32 |
| 484 | 39117_at | 0.557 | PHF2 | 9q22.31 |
| 485 | 34320_at | 0.557 | PTRF | 17q21.2 |
| 486 | 36791_g_at | 0.556 | TPM1 | 15q22.1 |
| 487 | 539_at | 0.556 | RYK | 3q22 |
| 488 | 40825_at | 0.556 | MAPRE3 | 2p23.3-p23.1 |
| 489 | 32169_at | 0.556 | FBXO21 | 12q24.21 |
| 490 | 38782_at | 0.555 | GTF2H1 | 11p15.1-p14 |
| 491 | 1677_at | 0.555 | IGFBP5 | 2q33-q36 |
| 492 | 33899_at | 0.555 | ALDH9A1 | 1q23.1 |
| 493 | 40843_at | 0.555 | ICAP-1A | 2p25.2 |
| 494 | 32172_at | 0.554 | SHARP | 1p36.33-p36.11 |
| 495 | 35303_at | 0.554 | INSIG1 | 7q36 |
| 496 | 34235_at | 0.554 | GPR116 | 6p12.3 |
| 497 | 818_s_at | 0.554 | ATRX | Xq13.1-q21.1 |
| 498 | 33113_at | 0.554 | CITED2 | 6q23.3 |
| 499 | 34287_at | 0.553 | C21orf80 | 21q22.3 |
| 500 | 33418_at | 0.553 | cDNA DKFZp434A012 | |
| 501 | 36790_at | 0.552 | TPM1 | 15q22.1 |
| 502 | 40811_at | 0.552 | COASTER | 6p11.1 |
| 503 | 41739_s_at | 0.552 | CALD1 | 7q33 |
| 504 | 509_at | 0.551 | MADH4 | 18q21.1 |
| 505 | 37598_at | 0.551 | RASSF2 | 20pter-p12.1 |
| 506 | 36629_at | 0.55 | DSIPI | Xq22.3 |
| 507 | 41462_at | 0.55 | SNX2 | 5q23 |
| 508 | 36032_at | 0.55 | ? | |
| 509 | 39045_at | 0.549 | FLJ21432 | 12p13.31 |
| 510 | 36577_at | 0.549 | MIG2 | 14q22.1 |
| 511 | 39557_at | 0.549 | cDNA FLJ31246 fis | |
| 512 | 33819_at | 0.549 | LDHB | 12p12.2-p12.1 |
| 513 | 38610_s_at | 0.549 | KRT10 | 17q21-q23 |
| 514 | 890_at | 0.548 | UBE2A | Xq24-q25 |
| 515 | 32730_at | 0.548 | KIAA1750 | 8q22.1 |
| 516 | 1252_at | 0.548 | DP1 | 5q22-q23 |
| 517 | 32239_at | 0.548 | MATN2 | 8q22 |
| 518 | 33405_at | 0.548 | CAP2 | 6p22.2 |
| 519 | 37266_at | 0.548 | ZNF32 | 10q22-q25 |
| 520 | 39686_g_at | 0.548 | E46L | 22q13.31 |
| 521 | 40155_at | 0.547 | ABLIM1 | 10q25 |
| 522 | 35988_i_at | 0.547 | MYST1 | 16p11.1 |
| 523 | 34314_at | 0.547 | RRM1 | 11p15.5 |
| 524 | 35213_at | 0.546 | WBP4 | 13q13.3 |
| 525 | 37676_at | 0.546 | PDE8A | 15q25.1 |
| 526 | 39545_at | 0.546 | CDKN1C | 11p15.5 |
| 527 | 37708_r_at | 0.545 | ADH5 | 4q21-q25 |
| 528 | 41686_s_at | 0.545 | KIAA0752 | 5q35.3 |
| 529 | 202_at | 0.545 | HSF2 | 6q22.33 |
| 530 | 33399_at | 0.544 | ? | |
| 531 | 39380_at | 0.544 | GTAR | 4q13.3 |
| 532 | 35166_at | 0.544 | DSCR3 | 21q22.2 |
| 533 | 39693_at | 0.544 | MGC5508 | 11q13.1 |
| 534 | 149_at | 0.544 | DDX39 | 19p13.13 |
| 535 | 40522_at | 0.544 | GLUL | 1q31 |
| 536 | 40831_at | 0.544 | DKFZP586B0923 | 10q22.2 |
| 537 | 32253_at | 0.544 | RERE | 1p36.1-p36.2 |
| 538 | 1836_at | 0.543 | CCNI | 4q13.3 |
| 539 | 36991_at | 0.543 | SFRS4 | 1p35.2 |
| 540 | 171_at | 0.543 | VBP1 | Xq28 |
| 541 | 38508_s_at | 0.542 | CREBL1 | 6p21.3 |
| 542 | 33856_at | 0.542 | CXX1 | Xq26 |
| 543 | 36118_at | 0.542 | NCOA1 | 2p23 |
| 544 | 32038_s_at | 0.542 | SRP46 | 11q22 |
| 545 | 36964_at | 0.542 | MBTPS1 | 16q24 |
| 546 | 37005_at | 0.541 | NBL1 | 1p36.3-p36.2 |
| 547 | 34259_at | 0.541 | KIAA0664 | 17p13.3 |
| 548 | 1725_s_at | 0.541 | Oncogene E6-Ap, | |
| | | | Papillomavirus | |
| 549 | 34344_at | 0.541 | IKBKAP | 9q34 |
| 550 | 33303_at | 0.54 | SSPN | 12p11.2 |
| 551 | 32215_i_at | 0.54 | RHOBTB3 | 5q21.2 |
| 552 | 34675_at | 0.54 | cDNA FLJ13555 fis | |
| 553 | 718_at | 0.54 | PRSS11 | 10q26.3 |
| 554 | 35168_f_at | 0.54 | COL16A1 | 1p35-p34 |
| 555 | 33875_at | 0.539 | ATP6V0E | 5q35.2 |
| 556 | 32548_at | 0.539 | TEBP | 12 |
| 557 | 38980_at | 0.539 | MAP3K7IP2 | 6q25.1-q25.3 |
| 558 | 40998_at | 0.539 | TNRC11 | Xq13 |
| 559 | 31907_at | 0.539 | RPL14 | 3p22-p21.2 |
| 560 | 41770_at | 0.539 | MAOA | Xp11.4-p11.3 |
| 561 | 31605_at | 0.539 | LOC171220 | 12p13 |
| 562 | 34684_at | 0.538 | RECQL | 12p12 |
| 563 | 41872_at | 0.538 | DFNA5 | 7p15 |
| 564 | 34853_at | 0.538 | FLRT2 | 14q24-q32 |
| 565 | 40467_at | 0.537 | SDHD | 11q23 |
| 566 | 39405_at | 0.537 | KIAA0266 | 13q12.2-q13.3 |
| 567 | 36925_at | 0.537 | HSPA2 | 14q24.1 |
| 568 | 32564_at | 0.537 | SEC61B | 9q22.32-q31.3 |
| 569 | 33431_at | 0.536 | FMOD | 1q32 |
| 570 | 37248_at | 0.536 | CPZ | 4p16.1 |
| 571 | 39931_at | 0.535 | DYRK3 | 1q32 |
| 572 | 35753_at | 0.535 | PRPF8 | 17p13.3 |
| 573 | 41713_at | 0.535 | ZNF36 | 7q21.3-q22.1 |
| 574 | 32171_at | 0.535 | EIF5 | 14q32.33 |
| 575 | 1675_at | 0.535 | RASA1 | 5q13.3 |
| 576 | 35644_at | 0.535 | HEPH | Xq11-q12 |
| 577 | 32569_at | 0.534 | PAFAH1B1 | 17p13.3 |
| 578 | 34370_at | 0.533 | ARCN1 | 11q23.3 |
| 579 | 38011_at | 0.533 | C19orf2 | 19q12 |
| 580 | 41194_at | 0.533 | SRP14 | 15q22 |
| 581 | 39509_at | 0.533 | PDCD4 | 10q24 |
| 582 | 32143_at | 0.532 | OSR2 | 8q22.1 |
| 583 | 40634_at | 0.532 | NAP1L1 | 12q14.1 |
| 584 | 34255_at | 0.531 | DGAT1 | 8qter |
| 585 | 1101_at | 0.531 | APBB1 | 11p15 |
| 586 | 35999_r_at | 0.531 | KIAA0781 | 11q23.2 |
| 587 | 40083_at | 0.531 | KIAA0625 | 9q34.3 |
| 588 | 663_at | 0.531 | EIF1A | X |
| 589 | 39884_g_at | 0.531 | HSA9761 | 5q11-q14 |
| 590 | 1467_at | 0.531 | EPS8 | 12q23-q24 |
| 591 | 31866_at | 0.529 | PD2 | 19q13.1 |
| 592 | 1512_at | 0.529 | DYRK1A | 21q22.13 |
| 593 | 39897_at | 0.529 | KIAA1966 | 4q13.1 |
| 594 | 38385_at | 0.529 | DSTN | 20p11.23 |
| 595 | 32170_g_at | 0.529 | FBXO21 | 12q24.21 |
| 596 | 1850_at | 0.529 | MLH1 | 3p21.3 |
| 597 | 39366_at | 0.529 | PPP1R3C | 10q23-q24 |
| 598 | 2062_at | 0.528 | IGFBP7 | 4q12 |
| 599 | 32765_f_at | 0.528 | PGCP | 8q22.2 |
| 600 | 38035_at | 0.528 | MTMR6 | 13q12 |
| 601 | 37352_at | 0.528 | SP100 | 2q36.1 |
| 602 | 36169_at | 0.528 | NDUFA1 | Xq24 |
| 603 | 37707_i_at | 0.528 | ADH5 | 4q21-q25 |
| 604 | 41743_i_at | 0.528 | OPTN | 10p12.33 |
| 605 | 34890_at | 0.527 | ATP6V1A1 | 3q13.31 |
| 606 | 38351_at | 0.527 | cDNA DKFZp586L0120 | |
| 607 | 38990_at | 0.526 | ICK | 6p12.3-p11.2 |
| 608 | 37389_at | 0.526 | SMAP | 11p15.1 |
| 609 | 34445_at | 0.526 | KIAA0471 | 1q24-q25 |
| 610 | 40859_at | 0.526 | FLJ11806 | 14q31.3 |
| 611 | 37029_at | 0.526 | ATP5O | 21q22.11 |
| 612 | 41490_at | 0.525 | PRPS2 | Xp22.3-p22.2 |
| 613 | 39687_at | 0.525 | E46L | 22q13.31 |
| 614 | 35247_at | 0.525 | SNAPC5 | |
| 615 | 34417_at | 0.525 | cDNA DKFZp586E1120 | |
| 616 | 40105_at | 0.524 | MUT | 6p21 |
| 617 | 41379_at | 0.524 | SMC5 | 9q21.12 |
| 618 | 38059_g_at | 0.524 | DPT | 1q12-q23 |
| 619 | 39517_at | 0.524 | HTGN29 | 5q31.1 |
| 620 | 38743_f_at | 0.524 | RAF1 | 3p25 |
| 621 | 41746_at | 0.523 | NHP2L1 | 22q13.2-q13.31 |
| 622 | 507_s_at | 0.523 | ELF2 | 4q28 |
| 623 | 36423_at | 0.523 | P8 | 16p11.2 |
| 624 | 40988_at | 0.522 | YME1L1 | 10p14 |
| 625 | 34680_s_at | 0.521 | KIAA0107 | 3p14.3 |
| 626 | 40962_s_at | 0.521 | SMARCA2 | 9p22.3 |
| 627 | 36792_at | 0.521 | TPM1 | 15q22.1 |
| 628 | 40191_s_at | 0.52 | KIAA0582 | 2p14 |
| 629 | 37367_at | 0.52 | ATP6V1E1 | 22q11.1 |
| 630 | 35221_at | 0.52 | PURA | 5q31 |
| 631 | 38649_at | 0.52 | KIAA0970 | 13q14.11 |
| 632 | 34740_at | 0.52 | FOXO3A | 6q21 |
| 633 | 41300_s_at | 0.519 | ITM2B | 13q14.3 |
| 634 | 40239_g_at | 0.519 | MGC35048 | 16p13.13 |
| 635 | 36829_at | 0.518 | PER1 | 17p13.1-17p12 |
| 636 | 869_at | 0.518 | GTF2A2 | 15q21.2 |
| 637 | 32611_at | 0.518 | PBP | 12q24.22 |
| 638 | 37672_at | 0.518 | USP7 | 16p13.3 |
| 639 | 36533_at | 0.517 | PTGIS | 20q13.11-q13.13 |
| 640 | 40438_at | 0.517 | PPP1R12A | 12q15-q21 |
| 641 | 39118_at | 0.517 | DNAJA1 | 9p13-p12 |
| 642 | 39555_at | 0.517 | ING1L | 4q35.1 |
| 643 | 38518_at | 0.517 | SCML2 | Xp22 |
| 644 | 37027_at | 0.516 | AHNAK | 11q12-q13 |
| 645 | 40260_g_at | 0.516 | RBM9 | 22q13.1 |
| 646 | 147_at | 0.516 | TSG101 | 11p15 |
| 647 | 37616_at | 0.516 | AUH | 9q22.33 |
| 648 | 39809_at | 0.516 | HBP1 | 7q31.1 |
| 649 | 32119_at | 0.515 | cDNA DKFZp586B211 | |
| 650 | 35730_at | 0.515 | ADH1B | 4q21-q23 |
| 651 | 36095_at | 0.513 | CLIPR-59 | 19q13.13 |
| 652 | 38654_at | 0.513 | HNRPU | 1q43 |
| 653 | 41768_at | 0.513 | PRKAR1A | 17q23-q24 |
| 654 | 38627_at | 0.512 | HLF | 17q22 |
| 655 | 487_g_at | 0.512 | CASP9 | 1p36.3-p36.1 |
| 656 | 33244_at | 0.512 | CHN2 | 7p15.3 |
| 657 | 38724_at | 0.512 | KIAA0515 | 9q34.2 |
| 658 | 39740_g_at | 0.512 | NACA | 12q23-q24.1 |
| 659 | 33850_at | 0.512 | MAP4 | 3p21 |
| 660 | 35304_at | 0.511 | RAB6A | 11q13.3 |
| 661 | 36330_at | 0.511 | CCBL1 | 9q34.13 |
| 662 | 33240_at | 0.511 | SEMACAP3 | 3p13 |
| 663 | 32745_at | 0.511 | MRPL40 | 22q11.21 |
| 664 | 274_at | 0.51 | ZNF148 | 3q21 |
| 665 | 34792_at | 0.51 | AHCYL1 | 1p12 |
| 666 | 34781_at | 0.509 | DCTN6 | 8p12-p11 |
| 667 | 38626_at | 0.509 | KIAA0399 | 17p13.3 |
| 668 | 38812_at | 0.509 | LAMB2 | 3p21 |
| 669 | 35736_at | 0.508 | GRINL1A | 15q22.1 |
| 670 | 37359_at | 0.507 | KIAA0102 | 11q13.3 |
| 671 | 1873_at | 0.507 | XPC | 3p25 |
| 672 | 35258_f_at | 0.507 | SFRS2IP | 12p11.21 |
| 673 | 40854_at | 0.506 | UQCRC2 | 16p12 |
| 674 | 40064_at | 0.506 | ALS2CR3 | 2q33 |
| 675 | 37407_s_at | 0.506 | MYH11 | 16p13.13-p13.12 |
| 676 | 33444_at | 0.505 | M17S2 | 17q21.1 |
| 677 | 38826_at | 0.505 | 6-Sep | Xq24 |
| 678 | 40832_s_at | 0.505 | LAP1B | 1q24.2 |
| 679 | 1195_s_at | 0.505 | ICAP-1A | 2p25.2 |
| 680 | 35142_at | 0.504 | DKFZP564D172 | 5q14.3 |
| 681 | 38479_at | 0.504 | ANP32B | 9q22.32 |
| 682 | 37025_at | 0.504 | PIG7 | 16p13.3-p12 |
| 683 | 969_s_at | 0.503 | USP9X | Xp11.4 |
| 684 | 39739_at | 0.502 | NACA | 12q23-q24.1 |
| 685 | 41195_at | 0.502 | LPP | 3q27-q28 |
| 686 | 32576_at | 0.502 | EIF3S5 | 11 |
| 687 | 41242_at | 0.502 | UAP1 | 1q23.1 |
| 688 | 35301_at | 0.502 | cDNA DKFZp564E2222 | |
| 689 | 33380_at | 0.501 | POLS | 5p15 |
| 690 | 37221_at | 0.501 | PRKAR2B | 7q22-q31.1 |
| 691 | 33916_at | 0.501 | NISCH | 3p21.1 |
| 692 | 37895_at | 0.5 | SLC35A1 | 6q16.1 |
| 693 | 1120_at | 0.5 | GSTM3 | 1p13.3 |
| 694 | 31463_s_at | 0.5 | ? | |
| 695 | 36899_at | 0.499 | SATB1 | 3p23 |
| 696 | 897_at | 0.497 | PKD1 | 16p13.3 |
| 697 | 41174_at | 0.497 | RANBP2L1 | 2q12.3 |
| 698 | 38106_at | 0.497 | YR-29 | 5q13.3-q14.1 |
| 699 | 38673_s_at | 0.497 | CDKN1C | 11p15.5 |
| 700 | 41400_at | 0.497 | TK1 | 17q23.2-q25.3 |
| 701 | 41283_at | 0.496 | HNRPH3 | 10q22 |
| 702 | 33835_at | 0.496 | KIAA0721 | 6q22.31 |
| 703 | 34359_at | 0.496 | CGI-130 | 6q13-q24.3 |
| 704 | 38875_r_at | 0.495 | GREB1 | 2p25.1 |
| 705 | 40096_at | 0.495 | ATP5A1 | 18q12-q21 |
| 706 | 37529_at | 0.494 | CACNA1H | 16p13.3 |
| 707 | 39418_at | 0.493 | DKFZP564M182 | 16p13.3 |
| 708 | 31897_at | 0.493 | DOC1 | 3q12.3 |
| 709 | 37381_g_at | 0.493 | GTF2B | 1p22-p21 |
| 710 | 40280_at | 0.492 | B7 | 12p13 |
| 711 | 40136_at | 0.492 | KIAA0676 | 5q35.3 |
| 712 | 506_s_at | 0.492 | STAT5A | 17q11.2 |
| 713 | 35812_at | 0.492 | TRN-SR | 7q31.1 |
| 714 | 41853_at | 0.491 | PRPSAP2 | 17p11.2-p12 |
| 715 | 37022_at | 0.491 | PRELP | 1q32 |
| 716 | 38079_at | 0.49 | GNG12 | 1p31.2 |
| 717 | 36149_at | 0.489 | DPYSL3 | 5q32 |
| 718 | 31880_at | 0.489 | D8S2298E | 8p12-p11.2 |
| 719 | 37199_at | 0.488 | CGI-60 | 2p25.1-p24.1 |
| 720 | 37671_at | 0.488 | LAMA4 | 6q21 |
| 721 | 31573_at | 0.488 | RPS25 | 11q23.3 |
| 722 | 39696_at | 0.488 | PEG10 | 7q21 |
| 723 | 39723_at | 0.487 | CUL1 | 7q34-q35 |
| 724 | 1074_at | 0.486 | RAB1A | 2p14 |
| 725 | 32175_at | 0.486 | CDC10 | 7p14.3-p14.1 |
| 726 | 35364_at | 0.486 | APPBP1 | 16q22 |
| 727 | 39019_at | 0.486 | LAPTM4A | 2p24.3 |
| 728 | 41772_at | 0.485 | MAOA | Xp11.4-p11.3 |
| 729 | 31670_s_at | 0.485 | CAMK2G | 10q22 |
| 730 | 33426_at | 0.484 | CHGB | 20pter-p12 |
| 731 | 34393_r_at | 0.483 | RAB1A | 2p14 |
| 732 | 40903_at | 0.483 | ATP6IP2 | Xq21 |
| 733 | 41251_at | 0.483 | TRIP3 | 17q21.1 |
| 734 | 39054_at | 0.482 | GSTM1 | 1p13.3 |
| 735 | 33912_at | 0.482 | ZMPSTE24 | 1p34 |
| 736 | 40709_at | 0.482 | ZNF271 | 18q12 |
| 737 | 38662_at | 0.482 | BCRP1 | 14q24.1 |
| 738 | 32755_at | 0.481 | ACTA2 | 10q23.3 |
| 739 | 39741_at | 0.481 | HADHB | 2p23 |
| 740 | 35169_at | 0.481 | COL16A1 | 1p35-p34 |
| 741 | 40555_at | 0.48 | TC10 | 2p21 |
| 742 | 315_at | 0.48 | PRDM2 | 1p36 |
| 743 | 39180_at | 0.48 | FUS | 16p11.2 |
| 744 | 35253_at | 0.479 | GAB2 | 11q13.4 |
| 745 | 31536_at | 0.479 | RTN4 | 2p14-p13 |
| 746 | 767_at | 0.478 | MYH11 | 16p13.13-p13.12 |
| 747 | 39797_at | 0.477 | KIAA0349 | 6p21.1 |
| 748 | 32854_at | 0.477 | FBXW1B | 5q35.1 |
| 749 | 41191_at | 0.477 | KIAA0992 | 4q32.3 |
| 750 | 1151_at | 0.476 | RPL22 | 1p36.3-p36.2 |
| 751 | 35294_at | 0.476 | SSA2 | 1q31 |
| 752 | 1708_at | 0.475 | MAPK10 | 4q22.1-q23 |
| 753 | 39031_at | 0.475 | COX7A1 | 19q13.1 |
| 754 | 37747_at | 0.475 | ANXA5 | 4q28-q32 |
| 755 | 38433_at | 0.475 | AXL | 19q13.1 |
| 756 | 38049_g_at | 0.473 | RBPMS | 8p12-p11 |
| 757 | 36595_s_at | 0.473 | GATM | 15q14 |
| 758 | 39124_r_at | 0.473 | TRPC1 | 3q22-q24 |
| 759 | 706_at | 0.473 | NR3C1 | 5q31 |
| 760 | 37399_at | 0.47 | AKR1C3 | 10p15-p14 |
| 761 | 38265_at | 0.47 | RBBP6 | 16p12-p11.2 |
| 762 | 32563_at | 0.469 | ATP1B3 | 3q22-q23 |
| 763 | 37734_at | 0.469 | DIP2 | 21q22.3 |
| 764 | 39245_at | 0.469 | ? | |
| 765 | 40618_at | 0.468 | H41 | 3q22.2 |
| 766 | 31886_at | 0.467 | NT5E | 6q14-q21 |
| 767 | 41807_at | 0.467 | cDNA FLJ31959 fis | |
| 768 | 1447_at | 0.467 | PSMB1 | 6q27 |
| 769 | 41227_at | 0.466 | OCRL | Xq25-q26.1 |
| 770 | 774_g_at | 0.465 | MYH11 | 16p13.13-p13.12 |
| 771 | 35794_at | 0.465 | EFA6R | 8pter-p23.3 |
| 772 | 32254_at | 0.464 | FSTL3 | 19p13 |
| 773 | 38694_at | 0.464 | KIAA0738 | 7q33 |
| 774 | 34391_at | 0.464 | IGBP1 | Xq13.1-q13.3 |
| 775 | 39183_at | 0.463 | PCTK1 | Xp11.3-p11.23 |
| 776 | 31894_at | 0.463 | CENPC1 | 4q12-q13.3 |
| 777 | 39260_at | 0.462 | SLC16A4 | 1p13.1 |
| 778 | 232_at | 0.462 | LAMC1 | 1q31 |
| 779 | 1380_at | 0.461 | FGF7 | 15q15-q21.1 |
| 780 | 41737_at | 0.46 | SRRM1 | 1p36.11 |
| 781 | 36851_g_at | 0.46 | N33 | 8p22 |
| 782 | 41488_at | 0.459 | LOC57149 | 16p11.2 |
| 783 | 39623_at | 0.457 | NDP | Xp11.4 |
| 784 | 36970_at | 0.456 | KIAA0182 | 16q24.1 |
| 785 | 40086_at | 0.456 | KIAA0261 | 10q23.31-q23.32 |
| 786 | 33421_s_at | 0.455 | SC5DL | 11q23.3 |
| 787 | 40047_at | 0.454 | SBB103 | 12q12 |
| 788 | 39420_at | 0.452 | DDIT3 | 12q13.1-q13.2 |
| 789 | 37951_at | 0.451 | DLC1 | 8p22-p21.3 |
| 790 | 855_at | 0.45 | PDCD2 | 6q27 |
| 791 | 35739_at | 0.45 | MTMR3 | 22q12.2 |
| 792 | 33451_s_at | 0.449 | RPL22 | 1p36.3-p36.2 |
| 793 | 1278_at | 0.448 | AXL | 19q13.1 |
| 794 | 38542_at | 0.445 | ? | |
| 795 | 31896_at | 0.44 | NAG | 2p24 |
| 796 | 33341_at | 0.439 | GNB1 | 1p36.33 |
| 797 | 1846_at | 0.439 | LGALS8 | 1q42-q43 |
| 798 | 288_s_at | 0.438 | LBR | 1q42.1 |
| 799 | 31672_g_at | 0.438 | RBMS1P | |
| 800 | 36120_at | 0.437 | FVT1 | 18q21.3 |
| 801 | 38371_at | 0.437 | PSMA1 | 11p15.1 |
| 802 | 31812_at | 0.436 | GMPR | 6p23 |
| 803 | 35311_at | 0.436 | CREG | 1q24 |
| 804 | 41837_at | 0.436 | DKFZp761F2014 | 14q32.2 |
| 805 | 39775_at | 0.436 | SERPING1 | 11q12-q13.1 |
| 806 | 37765_at | 0.433 | LMOD1 | 1q32 |
| 807 | 39733_at | 0.431 | HERPUD1 | 16q12.2-q13 |
| 808 | 38075_at | 0.43 | SYPL | 7q11.23 |
| 809 | 41289_at | 0.43 | NCAM1 | 11q23.1 |
| 810 | 38459_g_at | 0.429 | CYB5 | 18q23 |
| 811 | 40461_at | 0.427 | TIX1 | 20q12 |
| 812 | 33220_at | 0.427 | ZNF187 | 6p21.31 |
| 813 | 32769_at | 0.426 | ALFY | 4q21.23 |
| 814 | 1394_at | 0.425 | ARHA | 3p21.3 |
| 815 | 35720_at | 0.422 | KIAA0893 | 1p13.2 |
| 816 | 34366_g_at | 0.42 | PPIE | 1p32 |
| 817 | 38737_at | 0.418 | IGF1 | 12q22-q23 |
| 818 | 38326_at | 0.417 | G0S2 | 1q32.2-q41 |
| 819 | 34378_at | 0.417 | ADFP | 9p21.2 |
| 820 | 38458_at | 0.417 | CYB5 | 18q23 |
| 821 | 35286_r_at | 0.415 | RY1 | 2p12.1 |
| 822 | 37309_at | 0.413 | ARHA | 3p21.3 |
| 823 | 36634_at | 0.412 | BTG2 | 1q32 |
| 824 | 753_at | 0.412 | NID2 | 14q21-q22 |
| 825 | 37195_at | 0.411 | CYP11A | 15q23-q24 |
| 826 | 37536_at | 0.409 | CD83 | 6p23 |
| 827 | 32066_g_at | 0.407 | CREM | 10p12.1-p11.1 |
| 828 | 41759_at | 0.403 | SKP1A | 5q31 |
| 829 | 36968_s_at | 0.403 | OIP2 | 13q13.1 |
| 830 | 40471_at | 0.402 | PXF | 1q22 |
| 831 | 35740_at | 0.397 | EMILIN | 2p23.3-p23.2 |
| 832 | 32242_at | 0.394 | DKFZp566K192 | |
| 833 | 1596_g_at | 0.393 | TEK | 9p21 |
| 834 | 34785_at | 0.392 | KIAA1025 | 12q24.21 |
| 835 | 37718_at | 0.391 | SNRK | 3p21.31 |
| 836 | 37701_at | 0.391 | RGS2 | 1q31 |
| 837 | 33756_at | 0.39 | AOC3 | 17q21 |
| 838 | 40621_at | 0.389 | PAWR | 12q21 |
| 839 | 583_s_at | 0.387 | VCAM1 | 1p32-p31 |
| 840 | 34793_s_at | 0.387 | PLS3 | Xq24 |
| 841 | 39163_at | 0.385 | KIDINS220 | 2q24 |
| 842 | 36681_at | 0.384 | APOD | 3q26.2-qter |
| 843 | 37623_at | 0.384 | NR4A2 | 2q22-q23 |
| 844 | 38054_at | 0.382 | HBXIP | 1p13.1 |
| 845 | 33848_r_at | 0.38 | CDKN1B | 12p13.1-p12 |
| 846 | 280_g_at | 0.377 | NR4A1 | 12q13 |
| 847 | 1787_at | 0.374 | CDKN1C | 11p15.5 |
| 848 | 37694_at | 0.373 | PHF3 | |
| 849 | 36458_at | 0.372 | KIAA1018 | 15q12 |
| 850 | 32849_at | 0.371 | SMC1L1 | Xp11.22-p11.21 |
| 851 | 39046_at | 0.371 | H2AV | 7p13 |
| 852 | 36974_at | 0.37 | PSMF1 | 20p12.2-p13 |
| 853 | 547_s_at | 0.37 | NR4A2 | 2q22-q23 |
| 854 | 479_at | 0.37 | DAB2 | 5p13 |
| 855 | 1737_s_at | 0.366 | IGFBP4 | 17q12-q21.1 |
| 856 | 32847_at | 0.363 | MYLK | 3q21 |
| 857 | 37732_at | 0.363 | RYBP | 3p13 |
| 858 | 32184_at | 0.358 | LMO2 | 11p13 |
| 859 | 41046_s_at | 0.357 | ZNF261 | Xq13.1 |
| 860 | 40487_at | 0.356 | MC7 | 11p11.2 |
| 861 | 32067_at | 0.353 | CREM | 10p12.1-p11.1 |
| 862 | 39561_at | 0.352 | DNAL4 | 22q13.1 |
| 863 | 36569_at | 0.347 | TNA | 3p22-p21.3 |
| 864 | 39373_at | 0.345 | FADS1 | 11q12.2-q13.1 |
| 865 | 38466_at | 0.344 | CTSK | 1q21 |
| 866 | 34784_at | 0.336 | DJ37E16.5 | 22cen-q12.3 |
| 867 | 37842_at | 0.336 | HIC | 7q21.11 |
| 868 | 33255_at | 0.334 | NASP | 8q11.23 |
| 869 | 1005_at | 0.329 | DUSP1 | 5q34 |
| 870 | 41864_at | 0.329 | ? | |
| 871 | 1241_at | 0.327 | PTP4A2 | 1p35 |
| 872 | 38228_g_at | 0.323 | MITF | 3p14.1-p12.3 |
| 873 | 32340_s_at | 0.322 | NSEP1 | 1p34 |
| 874 | 38312_at | 0.316 | cDNA DKFZp564O222 | |
| 875 | 32313_at | 0.305 | TPM2 | 9p13.2-p13.1 |
| 876 | 773_at | 0.302 | MYH11 | 16p13.13-p13.12 |
| 877 | 36065_at | 0.301 | LDB2 | 4p16 |
| 878 | 39066_at | 0.29 | MFAP4 | 17p11.2 |
| 879 | 34826_at | 0.288 | SDHA | 5p15 |
| 880 | 38430_at | 0.286 | FABP4 | 8q21 |
| 881 | 31855_at | 0.274 | SRPX | Xp21.1 |
| 882 | 33440_at | 0.273 | TCF8 | 10p11.2 |
| 883 | 40856_at | 0.258 | SERPINF1 | 17p13.1 |
| 884 | 40282_s_at | 0.251 | DF | 19p13.3 |
| 885 | 36165_at | 0.234 | COX6C | 8q22-q23 |
| 886 | 36201_at | 0.234 | GLO1 | 6p21.3-p21.1 |
| 887 | 36521_at | 0.233 | DZIP1 | 13q32.1 |
| 888 | 36931_at | 0.225 | TAGLN | 11q23.2 |
| 889 | 32314_g_at | 0.222 | TPM2 | 9p13.2-p13.1 |
| 890 | 40824_at | 0.206 | RANBP16 | 8p21 |
| 891 | 33790_at | 0.203 | CCL14 | 17q11.2 |
| 892 | 38734_at | 0.198 | PLN | 6q22.1 |
| 893 | 39690_at | 0.182 | ALP | 4q35 |
| 894 | 31830_s_at | 0.176 | SMTN | 22q12.2 |
| 895 | 31831_at | 0.157 | SMTN | 22q12.2 |
| 896 | 34203_at | 0.15 | CNN1 | 19p13.2-p13.1 |
| 897 | 1197_at | 0.134 | ACTG2 | 2p13.1 |
| 898 | 38995_at | 0.114 | CLDN5 | 22q11.21 |
| 899 | 38994_at | 0.114 | SOCS2 | 12q |
| 900 | 36892_at | 0.042 | ITGA7 | 12q13 |

TABLE 10
Ranking of the Top 100 Probe Sets Based on PCC Values

| Probe Set | | | Probe set | | | Probe set | | |
|---|---|---|---|---|---|---|---|---|
| Name | R1 | R2 | name | R1 | R2 | name | R1 | R2 |
| 37628_at | 0.865 | 0.808 | 37529_at | 0.669 | 0.494 | 201_s_at | 0.577 | 0.571 |
| 41859_at | 0.865 | 0.877 | 32175_at | 0.669 | 0.486 | 774_g_at | 0.576 | 0.465 |
| 38120_at | 0.852 | 0.768 | 35753_at | 0.667 | 0.535 | 40998_at | 0.576 | 0.539 |
| 32664_at | 0.848 | 0.749 | 38875_r_at | 0.667 | 0.495 | 41772_at | 0.573 | 0.485 |
| 35717_at | 0.847 | 0.783 | 32779_s_at | 0.665 | 0.599 | 40522_at | 0.572 | 0.544 |
| 34257_at | 0.846 | 0.786 | 41385_at | 0.665 | 0.594 | 41768_at | 0.571 | 0.513 |
| 38220_at | 0.844 | 0.820 | 1319_at | 0.664 | 0.580 | 37828_at | 0.569 | 0.677 |
| 40423_at | 0.844 | 0.785 | 32593_at | 0.664 | 0.692 | 280_g_at | 0.569 | 0.377 |
| 38650_at | 0.840 | 0.748 | 38101_at | 0.663 | 0.615 | 33431_at | 0.568 | 0.536 |
| 38439_at | 0.829 | 0.736 | 39864_at | 0.663 | 0.698 | 1278_at | 0.566 | 0.448 |
| 39673_i_at | 0.827 | 0.698 | 39037_at | 0.663 | 0.726 | 35736_at | 0.566 | 0.508 |
| 38047_at | 0.822 | 0.756 | 32057_at | 0.663 | 0.667 | 37985_at | 0.565 | 0.608 |
| 1396_at | 0.821 | 0.756 | 38518_at | 0.663 | 0.517 | 38326_at | 0.565 | 0.417 |
| 37015_at | 0.820 | 0.751 | 39556_at | 0.662 | 0.725 | 37197_s_at | 0.562 | 0.647 |
| 40775_at | 0.818 | 0.666 | 40841_at | 0.661 | 0.618 | 41529_g_at | 0.560 | 0.620 |
| 32145_at | 0.817 | 0.786 | 39117_at | 0.660 | 0.557 | 39838_at | 0.560 | 0.582 |
| 35742_at | 0.815 | 0.708 | 36695_at | 0.659 | 0.602 | 2092_s_at | 0.560 | 0.679 |
| 1290_g_at | 0.814 | 0.776 | 39681_at | 0.659 | 0.690 | 31672_g_at | 0.559 | 0.438 |
| 39674_r_at | 0.811 | 0.699 | 1850_at | 0.659 | 0.529 | 37701_at | 0.559 | 0.391 |
| 36917_at | 0.810 | 0.752 | 38013_at | 0.657 | 0.672 | 40260_g_at | 0.558 | 0.516 |
| 1897_at | 0.809 | 0.770 | 1377_at | 0.656 | 0.605 | 33249_at | 0.558 | 0.618 |
| 755_at | 0.809 | 0.822 | 41739_s_at | 0.655 | 0.552 | 33198_at | 0.557 | 0.705 |
| 38176_at | 0.805 | 0.729 | 36095_at | 0.654 | 0.513 | 1708_at | 0.557 | 0.475 |
| 36073_at | 0.804 | 0.774 | 35169_at | 0.653 | 0.481 | 38116_at | 0.556 | 0.595 |
| 32764_at | 0.803 | 0.691 | 36533_at | 0.653 | 0.517 | 40832_s_at | 0.555 | 0.505 |
| 39750_at | 0.796 | 0.718 | 40698_at | 0.651 | 0.563 | 37958_at | 0.555 | 0.617 |
| 35645_at | 0.795 | 0.714 | 33936_at | 0.651 | 0.702 | 1787_at | 0.555 | 0.374 |
| 38717_at | 0.793 | 0.789 | 1507_s_at | 0.648 | 0.596 | 333_s_at | 0.554 | 0.562 |
| 37394_at | 0.791 | 0.656 | 38211_at | 0.648 | 0.681 | 36690_at | 0.554 | 0.654 |
| 36160_s_at | 0.788 | 0.846 | 38351_at | 0.648 | 0.527 | 40839_at | 0.553 | 0.611 |
| 36867_at | 0.785 | 0.739 | 32239_at | 0.647 | 0.548 | 32569_at | 0.553 | 0.534 |
| 39852_at | 0.783 | 0.738 | 32582_at | 0.646 | 0.578 | 33302_at | 0.553 | 0.619 |
| 37643_at | 0.779 | 0.716 | 31880_at | 0.646 | 0.489 | 1058_at | 0.552 | 0.668 |
| 40767_at | 0.779 | 0.640 | 38342_at | 0.646 | 0.632 | 38990_at | 0.552 | 0.526 |
| 41449_at | 0.778 | 0.762 | 33240_at | 0.645 | 0.511 | 506_s_at | 0.551 | 0.492 |
| 40488_at | 0.778 | 0.698 | 33136_at | 0.645 | 0.628 | 871_s_at | 0.550 | 0.630 |
| 40063_at | 0.777 | 0.729 | 39438_at | 0.645 | 0.690 | 33113_at | 0.549 | 0.554 |
| 41685_at | 0.777 | 0.871 | 38035_at | 0.644 | 0.528 | 507_s_at | 0.548 | 0.523 |
| 34163_g_at | 0.776 | 0.709 | 33278_at | 0.644 | 0.560 | 40876_at | 0.548 | 0.754 |
| 40570_at | 0.775 | 0.668 | 33140_at | 0.644 | 0.607 | 35754_at | 0.548 | 0.632 |
| 37446_at | 0.771 | 0.613 | 40145_at | 0.643 | 0.636 | 34287_at | 0.546 | 0.553 |
| 38669_at | 0.770 | 0.712 | 34215_at | 0.642 | 0.611 | 39775_at | 0.545 | 0.436 |
| 36627_at | 0.770 | 0.675 | 538_at | 0.642 | 0.622 | 41174_at | 0.545 | 0.497 |
| 1640_at | 0.769 | 0.734 | 35846_at | 0.641 | 0.655 | 34417_at | 0.545 | 0.525 |
| 35681_r_at | 0.769 | 0.644 | 39545_at | 0.641 | 0.546 | 34259_at | 0.544 | 0.541 |
| 36894_at | 0.768 | 0.634 | 32521_at | 0.641 | 0.563 | 38724_at | 0.544 | 0.512 |
| 41137_at | 0.768 | 0.631 | 34320_at | 0.640 | 0.557 | 32769_at | 0.543 | 0.426 |
| 39397_at | 0.768 | 0.700 | 39939_at | 0.640 | 0.593 | 33916_at | 0.541 | 0.501 |
| 41273_at | 0.766 | 0.758 | 40419_at | 0.639 | 0.599 | 38470_i_at | 0.541 | 0.565 |
| 38122_at | 0.761 | 0.707 | 36791_g_at | 0.638 | 0.556 | 38438_at | 0.540 | 0.612 |
| 40861_at | 0.760 | 0.689 | 40674_s_at | 0.638 | 0.697 | 31573_at | 0.540 | 0.488 |
| 35164_at | 0.758 | 0.621 | 32143_at | 0.638 | 0.532 | 31536_at | 0.539 | 0.479 |
| 39400_at | 0.758 | 0.634 | 41770_at | 0.636 | 0.539 | 39376_at | 0.539 | 0.715 |
| 872_i_at | 0.756 | 0.770 | 1909_at | 0.636 | 0.736 | 34859_at | 0.538 | 0.669 |
| 41738_at | 0.755 | 0.707 | 38581_at | 0.636 | 0.573 | 39733_at | 0.538 | 0.431 |
| 38113_at | 0.754 | 0.681 | 41227_at | 0.636 | 0.466 | 1211_s_at | 0.538 | 0.614 |
| 40202_at | 0.754 | 0.606 | 39790_at | 0.635 | 0.579 | 38059_g_at | 0.537 | 0.524 |
| 34760_at | 0.749 | 0.699 | 36091_at | 0.635 | 0.652 | 31510_s_at | 0.537 | 0.668 |
| 32526_at | 0.749 | 0.631 | 41191_at | 0.633 | 0.477 | 1596_g_at | 0.537 | 0.393 |
| 40994_at | 0.749 | 0.627 | 33817_at | 0.631 | 0.606 | 38711_at | 0.537 | 0.578 |
| 37908_at | 0.748 | 0.557 | 36790_at | 0.631 | 0.552 | 32676_at | 0.536 | 0.642 |
| 38119_at | 0.747 | 0.669 | 34162_at | 0.631 | 0.566 | 38768_at | 0.536 | 0.671 |
| 33690_at | 0.747 | 0.565 | 39691_at | 0.630 | 0.683 | 34819_at | 0.535 | 0.614 |
| 41478_at | 0.745 | 0.653 | 38049_g_at | 0.630 | 0.473 | 36629_at | 0.535 | 0.550 |
| 38634_at | 0.745 | 0.677 | 38754_at | 0.628 | 0.560 | 36578_at | 0.535 | 0.575 |
| 32109_at | 0.744 | 0.670 | 31605_at | 0.628 | 0.539 | 35335_at | 0.534 | 0.616 |
| 41049_at | 0.744 | 0.694 | 37230_at | 0.628 | 0.590 | 39687_at | 0.534 | 0.525 |
| 32778_at | 0.743 | 0.689 | 41300_s_at | 0.628 | 0.519 | 37532_at | 0.534 | 0.710 |
| 37599_at | 0.743 | 0.652 | 31897_at | 0.627 | 0.493 | 41634_at | 0.534 | 0.629 |
| 32076_at | 0.741 | 0.713 | 34789_at | 0.627 | 0.606 | 34821_at | 0.534 | 0.638 |
| 35234_at | 0.741 | 0.634 | 40508_at | 0.626 | 0.610 | 38627_at | 0.533 | 0.512 |
| 40853_at | 0.740 | 0.647 | 33303_at | 0.626 | 0.540 | 34445_at | 0.533 | 0.526 |
| 1731_at | 0.739 | 0.619 | 35221_at | 0.626 | 0.520 | 32755_at | 0.533 | 0.481 |
| 39714_at | 0.738 | 0.743 | 37638_at | 0.626 | 0.607 | 1578_g_at | 0.532 | 0.661 |
| 41505_r_at | 0.738 | 0.638 | 41744_at | 0.625 | 0.723 | 34349_at | 0.530 | 0.603 |
| 1761_at | 0.736 | 0.789 | 41405_at | 0.625 | 0.592 | 33426_at | 0.529 | 0.484 |
| 36577_at | 0.736 | 0.549 | 41594_at | 0.625 | 0.644 | 39441_at | 0.529 | 0.633 |
| 32805_at | 0.736 | 0.586 | 35782_at | 0.625 | 0.638 | 39066_at | 0.528 | 0.290 |
| 1577_at | 0.736 | 0.767 | 37221_at | 0.625 | 0.501 | 38318_at | 0.527 | 0.741 |
| 38643_at | 0.735 | 0.714 | 39147_g_at | 0.624 | 0.592 | 40438_at | 0.527 | 0.517 |
| 40496_at | 0.734 | 0.617 | 38727_at | 0.624 | 0.597 | 1090_f_at | 0.525 | 0.671 |
| 1135_at | 0.732 | 0.652 | 39109_at | 0.623 | 0.564 | 40077_at | 0.524 | 0.650 |
| 41138_at | 0.731 | 0.565 | 40399_r_at | 0.623 | 0.630 | 38079_at | 0.523 | 0.490 |
| 38968_at | 0.729 | 0.683 | 39436_at | 0.623 | 0.596 | 35811_at | 0.522 | 0.566 |
| 1327_s_at | 0.729 | 0.626 | 37710_at | 0.622 | 0.600 | 1530_g_at | 0.522 | 0.560 |
| 34772_at | 0.729 | 0.748 | 36727_at | 0.621 | 0.558 | 38690_at | 0.520 | 0.591 |
| 32535_at | 0.729 | 0.582 | 33911_at | 0.621 | 0.633 | 41147_at | 0.520 | 0.699 |
| 37743_at | 0.729 | 0.617 | 36634_at | 0.621 | 0.412 | 39693_at | 0.517 | 0.544 |
| 34355_at | 0.728 | 0.662 | 36118_at | 0.620 | 0.542 | 1147_at | 0.517 | 0.637 |
| 32259_at | 0.728 | 0.611 | 39019_at | 0.620 | 0.486 | 34808_at | 0.517 | 0.614 |
| 32251_at | 0.728 | 0.615 | 33244_at | 0.620 | 0.512 | 39055_at | 0.517 | 0.610 |
| 41000_at | 0.727 | 0.677 | 39557_at | 0.619 | 0.549 | 39986_at | 0.516 | 0.591 |
| 40786_at | 0.725 | 0.705 | 40962_s_at | 0.619 | 0.521 | 33380_at | 0.515 | 0.501 |
| 39701_at | 0.724 | 0.611 | 39829_at | 0.618 | 0.656 | 34793_s_at | 0.512 | 0.387 |
| 34740_at | 0.723 | 0.520 | 31852_at | 0.617 | 0.687 | 37842_at | 0.512 | 0.336 |
| 35354_at | 0.723 | 0.667 | 818_s_at | 0.617 | 0.554 | 39685_at | 0.511 | 0.568 |
| 34169_s_at | 0.722 | 0.661 | 37407_s_at | 0.617 | 0.506 | 38279_at | 0.511 | 0.587 |
| 36873_at | 0.722 | 0.633 | 36829_at | 0.617 | 0.518 | 41420_at | 0.510 | 0.593 |
| 39243_s_at | 0.721 | 0.702 | 35173_at | 0.617 | 0.774 | 547_s_at | 0.506 | 0.370 |
| 34877_at | 0.720 | 0.671 | 39846_at | 0.617 | 0.564 | 1252_at | 0.506 | 0.548 |
| 36119_at | 0.720 | 0.622 | 897_at | 0.616 | 0.497 | 32597_at | 0.506 | 0.648 |
| 38364_at | 0.719 | 0.599 | 40607_at | 0.616 | 0.708 | 32847_at | 0.505 | 0.363 |
| 39025_at | 0.718 | 0.746 | 32087_at | 0.616 | 0.672 | 1678_g_at | 0.504 | 0.638 |
| 32747_at | 0.717 | 0.706 | 36488_at | 0.615 | 0.636 | 35999_r_at | 0.504 | 0.531 |
| 36975_at | 0.717 | 0.696 | 487_g_at | 0.615 | 0.512 | 36964_at | 0.504 | 0.542 |
| 33443_at | 0.716 | 0.635 | 40617_at | 0.615 | 0.645 | 753_at | 0.504 | 0.412 |
| 32542_at | 0.716 | 0.662 | 853_at | 0.614 | 0.674 | 32153_s_at | 0.504 | 0.575 |
| 32765_f_at | 0.716 | 0.528 | 1101_at | 0.612 | 0.531 | 32668_at | 0.503 | 0.809 |
| 41013_at | 0.715 | 0.578 | 35359_at | 0.612 | 0.653 | 38394_at | 0.500 | 0.790 |
| 37707_i_at | 0.715 | 0.528 | 32851_at | 0.612 | 0.614 | 37623_at | 0.500 | 0.384 |
| 35785_at | 0.715 | 0.691 | 41195_at | 0.610 | 0.502 | 41759_at | 0.500 | 0.403 |
| 35783_at | 0.713 | 0.753 | 40825_at | 0.608 | 0.556 | 37027_at | 0.500 | 0.516 |
| 36515_at | 0.712 | 0.583 | 33235_at | 0.607 | 0.587 | 33756_at | 0.500 | 0.390 |
| 924_s_at | 0.711 | 0.640 | 40155_at | 0.606 | 0.547 | 1120_at | 0.498 | 0.500 |
| 33857_at | 0.710 | 0.630 | 37617_at | 0.606 | 0.636 | 40971_at | 0.497 | 0.581 |
| 35704_at | 0.709 | 0.656 | 40213_at | 0.606 | 0.615 | 1307_at | 0.497 | 0.582 |
| 41747_s_at | 0.709 | 0.587 | 39260_at | 0.606 | 0.462 | 35955_at | 0.496 | 0.640 |
| 35316_at | 0.709 | 0.743 | 39294_at | 0.606 | 0.595 | 38459_g_at | 0.494 | 0.429 |
| 38508_s_at | 0.708 | 0.542 | 2062_at | 0.605 | 0.528 | 38466_at | 0.493 | 0.344 |
| 35644_at | 0.707 | 0.535 | 31886_at | 0.605 | 0.467 | 32708_g_at | 0.493 | 0.627 |
| 35366_at | 0.707 | 0.560 | 38695_at | 0.605 | 0.715 | 36521_at | 0.493 | 0.233 |
| 37005_at | 0.706 | 0.541 | 767_at | 0.604 | 0.478 | 37315_f_at | 0.492 | 0.602 |
| 40961_at | 0.705 | 0.745 | 33878_at | 0.604 | 0.657 | 33440_at | 0.492 | 0.273 |
| 36948_at | 0.703 | 0.766 | 32119_at | 0.603 | 0.515 | 35843_at | 0.491 | 0.567 |
| 39743_at | 0.702 | 0.775 | 38228_g_at | 0.602 | 0.323 | 34235_at | 0.491 | 0.554 |
| 39369_at | 0.702 | 0.635 | 39979_at | 0.602 | 0.588 | 479_at | 0.490 | 0.370 |
| 36596_r_at | 0.701 | 0.575 | 35740_at | 0.601 | 0.397 | 40140_at | 0.490 | 0.666 |
| 39038_at | 0.700 | 0.576 | 32768_at | 0.601 | 0.648 | 41379_at | 0.490 | 0.524 |
| 851_s_at | 0.699 | 0.725 | 34637_f_at | 0.600 | 0.678 | 39623_at | 0.489 | 0.457 |
| 41655_at | 0.699 | 0.664 | 35794_at | 0.600 | 0.465 | 41277_at | 0.489 | 0.632 |
| 35246_at | 0.697 | 0.652 | 36792_at | 0.599 | 0.521 | 36458_at | 0.488 | 0.372 |
| 38317_at | 0.696 | 0.593 | 39366_at | 0.599 | 0.529 | 35166_at | 0.488 | 0.544 |
| 37294_at | 0.696 | 0.649 | 40770_f_at | 0.599 | 0.655 | 654_at | 0.488 | 0.643 |
| 35168_f_at | 0.696 | 0.540 | 38673_s_at | 0.598 | 0.497 | 38826_at | 0.488 | 0.505 |
| 36159_s_at | 0.696 | 0.589 | 1675_at | 0.598 | 0.535 | 33915_at | 0.487 | 0.599 |
| 35752_s_at | 0.695 | 0.642 | 33399_at | 0.598 | 0.544 | 38626_at | 0.487 | 0.509 |
| 35325_at | 0.694 | 0.577 | 32700_at | 0.598 | 0.592 | 38074_at | 0.486 | 0.634 |
| 718_at | 0.693 | 0.540 | 32337_at | 0.597 | 0.589 | 35730_at | 0.486 | 0.515 |
| 37708_r_at | 0.692 | 0.545 | 41872_at | 0.597 | 0.538 | 31932_f_at | 0.485 | 0.586 |
| 34363_at | 0.691 | 0.665 | 1836_at | 0.597 | 0.543 | 34803_at | 0.485 | 0.633 |
| 2086_s_at | 0.691 | 0.637 | 176_at | 0.596 | 0.654 | 32066_g_at | 0.484 | 0.407 |
| 37406_at | 0.690 | 0.665 | 37598_at | 0.595 | 0.551 | 37199_at | 0.484 | 0.488 |
| 41796_at | 0.689 | 0.779 | 41490_at | 0.593 | 0.525 | 33123_at | 0.483 | 0.605 |
| 35331_at | 0.689 | 0.565 | 37266_at | 0.593 | 0.548 | 36120_at | 0.483 | 0.437 |
| 1736_at | 0.688 | 0.688 | 39054_at | 0.592 | 0.482 | 35294_at | 0.483 | 0.476 |
| 32107_at | 0.688 | 0.598 | 31936_s_at | 0.591 | 0.565 | 39856_at | 0.482 | 0.620 |
| 34853_at | 0.688 | 0.538 | 1629_s_at | 0.591 | 0.692 | 1323_at | 0.482 | 0.604 |
| 36396_at | 0.688 | 0.785 | 40461_at | 0.588 | 0.427 | 33830_at | 0.481 | 0.679 |
| 32780_at | 0.687 | 0.594 | 39031_at | 0.588 | 0.475 | 38743_f_at | 0.481 | 0.524 |
| 32254_at | 0.687 | 0.464 | 1529_at | 0.588 | 0.586 | 41656_at | 0.480 | 0.661 |
| 37248_at | 0.687 | 0.536 | 39555_at | 0.588 | 0.517 | 38106_at | 0.479 | 0.497 |
| 33800_at | 0.687 | 0.579 | 1005_at | 0.588 | 0.329 | 39420_at | 0.479 | 0.452 |
| 38837_at | 0.686 | 0.584 | 38745_at | 0.587 | 0.702 | 39110_at | 0.478 | 0.566 |
| 39360_at | 0.685 | 0.640 | 35784_at | 0.587 | 0.622 | 31463_s_at | 0.478 | 0.500 |
| 41638_at | 0.683 | 0.633 | 706_at | 0.587 | 0.473 | 41686_s_at | 0.476 | 0.545 |
| 34198_at | 0.683 | 0.638 | 41620_at | 0.587 | 0.618 | 41289_at | 0.476 | 0.430 |
| 38033_at | 0.683 | 0.597 | 226_at | 0.587 | 0.657 | 34860_g_at | 0.476 | 0.593 |
| 1467_at | 0.682 | 0.531 | 35739_at | 0.586 | 0.450 | 38980_at | 0.475 | 0.539 |
| 40203_at | 0.682 | 0.660 | 37205_at | 0.586 | 0.645 | 36636_at | 0.475 | 0.590 |
| 38685_at | 0.680 | 0.665 | 2010_at | 0.585 | 0.732 | 36423_at | 0.474 | 0.523 |
| 35741_at | 0.680 | 0.619 | 37391_at | 0.585 | 0.567 | 36065_at | 0.473 | 0.301 |
| 39150_at | 0.680 | 0.569 | 38433_at | 0.585 | 0.475 | 1873_at | 0.473 | 0.507 |
| 37604_at | 0.679 | 0.573 | 39165_at | 0.584 | 0.710 | 35055_at | 0.472 | 0.586 |
| 38812_at | 0.679 | 0.509 | 1677_at | 0.584 | 0.555 | 38694_at | 0.471 | 0.464 |
| 32696_at | 0.678 | 0.604 | 1380_at | 0.583 | 0.461 | 35276_at | 0.471 | 0.575 |
| 38375_at | 0.678 | 0.713 | 34842_at | 0.583 | 0.759 | 33799_at | 0.469 | 0.607 |
| 38254_at | 0.678 | 0.601 | 35767_at | 0.583 | 0.662 | 773_at | 0.469 | 0.302 |
| 39082_at | 0.677 | 0.587 | 36825_at | 0.583 | 0.587 | 40211_at | 0.467 | 0.636 |
| 32215_i_at | 0.677 | 0.540 | 38385_at | 0.583 | 0.529 | 35303_at | 0.465 | 0.554 |
| 39582_at | 0.677 | 0.618 | 34675_at | 0.582 | 0.540 | 41283_at | 0.464 | 0.496 |
| 36915_at | 0.676 | 0.720 | 1737_s_at | 0.581 | 0.366 | 37195_at | 0.463 | 0.411 |
| 40576_f_at | 0.676 | 0.652 | 38982_at | 0.580 | 0.719 | 40811_at | 0.463 | 0.552 |
| 539_at | 0.675 | 0.556 | 41271_at | 0.580 | 0.560 | 1348_s_at | 0.463 | 0.718 |
| 37595_at | 0.674 | 0.618 | 36595_s_at | 0.579 | 0.473 | 32038_s_at | 0.462 | 0.542 |
| 33868_at | 0.673 | 0.637 | 38923_at | 0.578 | 0.605 | 33856_at | 0.462 | 0.542 |
| 37676_at | 0.673 | 0.546 | 37765_at | 0.578 | 0.433 | 36543_at | 0.461 | 0.648 |
| 39124_r_at | 0.673 | 0.473 | 37373_at | 0.578 | 0.710 | 37022_at | 0.461 | 0.491 |
| 38649_at | 0.673 | 0.520 | 37389_at | 0.578 | 0.526 | 38610_s_at | 0.459 | 0.549 |
| 41771_g_at | 0.673 | 0.687 | 38916_at | 0.578 | 0.651 | 33444_at | 0.459 | 0.505 |
| 227_g_at | 0.672 | 0.733 | 33899_at | 0.578 | 0.555 | 32713_at | 0.459 | 0.598 |

| Probe Set | | | Probe Set | | |
|---|---|---|---|---|---|
| Name | R1 | R2 | Name | R1 | R2 |
| 37486_f_at | 0.459 | 0.615 | 1848_at | 0.288 | 0.591 |
| 38802_at | 0.458 | 0.739 | 34203_at | 0.288 | 0.150 |
| 32695_at | 0.458 | 0.722 | 40280_at | 0.286 | 0.492 |
| 35142_at | 0.457 | 0.504 | 37359_at | 0.286 | 0.507 |
| 149_at | 0.457 | 0.544 | 33790_at | 0.285 | 0.203 |
| 39561_at | 0.457 | 0.352 | 38542_at | 0.283 | 0.445 |
| 33835_at | 0.457 | 0.496 | 33870_at | 0.281 | 0.589 |
| 32244_at | 0.455 | 0.576 | 869_at | 0.281 | 0.518 |
| 32313_at | 0.453 | 0.305 | 315_at | 0.277 | 0.480 |
| 37242_at | 0.452 | 0.753 | 36970_at | 0.275 | 0.456 |
| 38353_at | 0.451 | 0.628 | 38782_at | 0.275 | 0.555 |
| 32743_at | 0.451 | 0.577 | 39739_at | 0.275 | 0.502 |
| 36149_at | 0.449 | 0.489 | 41662_at | 0.275 | 0.661 |
| 38050_at | 0.449 | 0.599 | 37748_at | 0.274 | 0.617 |
| 39072_at | 0.448 | 0.662 | 33441_at | 0.272 | 0.561 |
| 32777_at | 0.447 | 0.743 | 35228_at | 0.272 | 0.624 |
| 38458_at | 0.447 | 0.417 | 32160_at | 0.269 | 0.576 |
| 37731_at | 0.446 | 0.602 | 41853_at | 0.268 | 0.491 |
| 38265_at | 0.446 | 0.470 | 41746_at | 0.268 | 0.523 |
| 41194_at | 0.445 | 0.533 | 32854_at | 0.268 | 0.477 |
| 40856_at | 0.445 | 0.258 | 1070_at | 0.267 | 0.601 |
| 36650_at | 0.444 | 0.612 | 35737_at | 0.266 | 0.583 |
| 39431_at | 0.443 | 0.622 | 232_at | 0.266 | 0.462 |
| 39380_at | 0.443 | 0.544 | 34785_at | 0.259 | 0.392 |
| 37131_at | 0.443 | 0.585 | 34792_at | 0.259 | 0.510 |
| 32576_at | 0.442 | 0.502 | 36542_at | 0.257 | 0.686 |
| 36991_at | 0.441 | 0.543 | 192_at | 0.255 | 0.643 |
| 324_f_at | 0.441 | 0.562 | 36330_at | 0.255 | 0.511 |
| 41837_at | 0.440 | 0.436 | 35364_at | 0.254 | 0.486 |
| 34753_at | 0.439 | 0.587 | 1846_at | 0.252 | 0.439 |
| 40083_at | 0.438 | 0.531 | 32172_at | 0.252 | 0.554 |
| 35936_g_at | 0.437 | 0.563 | 37616_at | 0.249 | 0.516 |
| 32067_at | 0.434 | 0.353 | 32822_at | 0.247 | 0.585 |
| 39045_at | 0.432 | 0.549 | 40105_at | 0.246 | 0.524 |
| 36489_at | 0.432 | 0.636 | 509_at | 0.245 | 0.551 |
| 41242_at | 0.431 | 0.502 | 37352_at | 0.244 | 0.528 |
| 1501_at | 0.431 | 0.576 | 36169_at | 0.243 | 0.528 |
| 39315_at | 0.430 | 0.659 | 34890_at | 0.242 | 0.527 |
| 32618_at | 0.430 | 0.786 | 34370_at | 0.242 | 0.533 |
| 40136_at | 0.427 | 0.492 | 40709_at | 0.240 | 0.482 |
| 36626_at | 0.426 | 0.577 | 33865_at | 0.236 | 0.599 |
| 32667_at | 0.426 | 0.588 | 38820_at | 0.235 | 0.598 |
| 37951_at | 0.426 | 0.451 | 35213_at | 0.235 | 0.546 |
| 36569_at | 0.426 | 0.347 | 36492_at | 0.235 | 0.583 |
| 32611_at | 0.425 | 0.518 | 35356_at | 0.233 | 0.619 |
| 34990_at | 0.424 | 0.688 | 35643_at | 0.230 | 0.677 |
| 32184_at | 0.424 | 0.358 | 33847_s_at | 0.229 | 0.574 |
| 38693_at | 0.424 | 0.623 | 34359_at | 0.229 | 0.496 |
| 38737_at | 0.423 | 0.418 | 39346_at | 0.228 | 0.573 |
| 34372_at | 0.423 | 0.564 | 39517_at | 0.227 | 0.524 |
| 32792_at | 0.423 | 0.612 | 202_at | 0.225 | 0.545 |
| 36681_at | 0.423 | 0.384 | 35720_at | 0.225 | 0.422 |
| 39091_at | 0.422 | 0.668 | 40859_at | 0.225 | 0.526 |
| 33126_at | 0.422 | 0.651 | 33343_at | 0.221 | 0.600 |
| 41333_at | 0.421 | 0.595 | 950_at | 0.221 | 0.653 |
| 41338_at | 0.420 | 0.605 | 32548_at | 0.219 | 0.539 |
| 38664_at | 0.419 | 0.672 | 35163_at | 0.219 | 0.676 |
| 37718_at | 0.419 | 0.391 | 38662_at | 0.218 | 0.482 |
| 38892_at | 0.418 | 0.642 | 39083_at | 0.213 | 0.575 |
| 39741_at | 0.417 | 0.481 | 41864_at | 0.211 | 0.329 |
| 40601_at | 0.417 | 0.680 | 39723_at | 0.210 | 0.487 |
| 39170_at | 0.417 | 0.561 | 36968_s_at | 0.207 | 0.403 |
| 32314_g_at | 0.417 | 0.222 | 32564_at | 0.206 | 0.537 |
| 34784_at | 0.416 | 0.336 | 35209_at | 0.205 | 0.652 |
| 35988_i_at | 0.416 | 0.547 | 41600_at | 0.204 | 0.571 |
| 39809_at | 0.416 | 0.516 | 39428_at | 0.203 | 0.630 |
| 31872_at | 0.415 | 0.630 | 40480_s_at | 0.198 | 0.761 |
| 33942_s_at | 0.412 | 0.735 | 39180_at | 0.198 | 0.480 |
| 37399_at | 0.411 | 0.470 | 41830_at | 0.197 | 0.647 |
| 37379_at | 0.410 | 0.684 | 37309_at | 0.196 | 0.413 |
| 37895_at | 0.407 | 0.500 | 1241_at | 0.195 | 0.327 |
| 32099_at | 0.407 | 0.570 | 38415_at | 0.189 | 0.593 |
| 32253_at | 0.405 | 0.544 | 288_s_at | 0.187 | 0.438 |
| 39989_at | 0.403 | 0.587 | 37891_at | 0.186 | 0.559 |
| 36980_at | 0.403 | 0.592 | 32745_at | 0.183 | 0.511 |
| 508_at | 0.402 | 0.589 | 38479_at | 0.182 | 0.504 |
| 38312_at | 0.402 | 0.316 | 39401_at | 0.181 | 0.699 |
| 39350_at | 0.401 | 0.727 | 36851_g_at | 0.181 | 0.460 |
| 37725_at | 0.401 | 0.578 | 39418_at | 0.180 | 0.493 |
| 39663_at | 0.400 | 0.565 | 31894_at | 0.177 | 0.463 |
| 39509_at | 0.398 | 0.533 | 31896_at | 0.175 | 0.440 |
| 34344_at | 0.398 | 0.541 | 32730_at | 0.173 | 0.548 |
| 33862_at | 0.398 | 0.615 | 40239_g_at | 0.169 | 0.519 |
| 39897_at | 0.397 | 0.529 | 2003_s_at | 0.169 | 0.652 |
| 39690_at | 0.396 | 0.182 | 32169_at | 0.165 | 0.556 |
| 729_i_at | 0.396 | 0.597 | 39696_at | 0.165 | 0.488 |
| 41400_at | 0.395 | 0.497 | 32240_at | 0.163 | 0.562 |
| 32841_at | 0.395 | 0.594 | 34684_at | 0.161 | 0.538 |
| 41807_at | 0.395 | 0.467 | 35301_at | 0.161 | 0.502 |
| 659_g_at | 0.391 | 0.641 | 38054_at | 0.160 | 0.382 |
| 33351_at | 0.389 | 0.664 | 41606_at | 0.159 | 0.562 |
| 1127_at | 0.386 | 0.697 | 40634_at | 0.158 | 0.532 |
| 33875_at | 0.384 | 0.539 | 37747_at | 0.157 | 0.475 |
| 39046_at | 0.383 | 0.371 | 40096_at | 0.156 | 0.495 |
| 36931_at | 0.382 | 0.225 | 36032_at | 0.155 | 0.550 |
| 41288_at | 0.381 | 0.641 | 41046_s_at | 0.153 | 0.357 |
| 31670_s_at | 0.380 | 0.485 | 35738_at | 0.140 | 0.755 |
| 40281_at | 0.380 | 0.562 | 35812_at | 0.140 | 0.492 |
| 911_s_at | 0.379 | 0.618 | 40064_at | 0.138 | 0.506 |
| 34570_at | 0.374 | 0.586 | 40047_at | 0.137 | 0.454 |
| 1725_s_at | 0.373 | 0.541 | 32340_s_at | 0.135 | 0.322 |
| 40282_s_at | 0.372 | 0.251 | 31944_at | 0.133 | 0.601 |
| 32662_at | 0.371 | 0.578 | 32242_at | 0.133 | 0.394 |
| 38985_at | 0.370 | 0.633 | 32170_g_at | 0.132 | 0.529 |
| 583_s_at | 0.370 | 0.387 | 38408_at | 0.132 | 0.656 |
| 38430_at | 0.369 | 0.286 | 36892_at | 0.115 | 0.042 |
| 35203_at | 0.367 | 0.566 | 171_at | 0.115 | 0.543 |
| 38734_at | 0.367 | 0.198 | 31866_at | 0.115 | 0.529 |
| 40432_at | 0.367 | 0.573 | 36925_at | 0.114 | 0.537 |
| 1151_at | 0.365 | 0.476 | 36620_at | 0.113 | 0.692 |
| 37671_at | 0.365 | 0.488 | 34774_at | 0.109 | 0.642 |
| 39797_at | 0.364 | 0.477 | 40039_g_at | 0.107 | 0.625 |
| 40618_at | 0.363 | 0.468 | 40824_at | 0.106 | 0.206 |
| 39740_g_at | 0.362 | 0.512 | 34314_at | 0.105 | 0.547 |
| 39097_at | 0.362 | 0.595 | 40621_at | 0.103 | 0.389 |
| 41251_at | 0.359 | 0.483 | 34393_r_at | 0.102 | 0.483 |
| 40916_at | 0.359 | 0.777 | 33103_s_at | 0.098 | 0.596 |
| 34255_at | 0.358 | 0.531 | 35311_at | 0.098 | 0.436 |
| 41701_at | 0.358 | 0.570 | 147_at | 0.093 | 0.516 |
| 36474_at | 0.358 | 0.569 | 33220_at | 0.091 | 0.427 |
| 31831_at | 0.358 | 0.157 | 969_s_at | 0.091 | 0.503 |
| 41713_at | 0.358 | 0.535 | 39884_g_at | 0.090 | 0.531 |
| 38994_at | 0.355 | 0.114 | 32849_at | 0.090 | 0.371 |
| 39686_g_at | 0.351 | 0.548 | 237_s_at | 0.089 | 0.639 |
| 33222_at | 0.350 | 0.626 | 41742_s_at | 0.087 | 0.642 |
| 36526_at | 0.350 | 0.584 | 31867_at | 0.084 | 0.602 |
| 40843_at | 0.347 | 0.555 | 39245_at | 0.083 | 0.469 |
| 31812_at | 0.346 | 0.436 | 41488_at | 0.082 | 0.459 |
| 450_g_at | 0.346 | 0.576 | 36974_at | 0.080 | 0.370 |
| 39931_at | 0.346 | 0.535 | 33912_at | 0.076 | 0.482 |
| 40831_at | 0.345 | 0.544 | 35286_r_at | 0.074 | 0.415 |
| 1719_at | 0.345 | 0.606 | 37734_at | 0.073 | 0.469 |
| 40471_at | 0.345 | 0.402 | 38011_at | 0.073 | 0.533 |
| 39118_at | 0.344 | 0.517 | 39715_at | 0.071 | 0.638 |
| 37107_at | 0.343 | 0.590 | 41136_s_at | 0.071 | 0.567 |
| 41462_at | 0.342 | 0.550 | 40467_at | 0.071 | 0.537 |
| 38075_at | 0.341 | 0.430 | 36899_at | 0.069 | 0.499 |
| 39163_at | 0.339 | 0.385 | 32171_at | 0.068 | 0.535 |
| 39405_at | 0.339 | 0.537 | 1195_s_at | 0.067 | 0.505 |
| 31830_s_at | 0.338 | 0.176 | 35318_at | 0.067 | 0.585 |
| 37367_at | 0.338 | 0.520 | 38839_at | 0.065 | 0.597 |
| 39731_at | 0.337 | 0.629 | 34356_at | 0.065 | 0.673 |
| 41691_at | 0.335 | 0.572 | 39033_at | 0.065 | 0.606 |
| 37381_g_at | 0.334 | 0.493 | 40854_at | 0.062 | 0.506 |
| 37706_at | 0.333 | 0.650 | 38654_at | 0.060 | 0.513 |
| 37694_at | 0.330 | 0.373 | 41743_i_at | 0.059 | 0.528 |
| 33341_at | 0.329 | 0.439 | 35247_at | 0.052 | 0.525 |
| 37715_at | 0.328 | 0.619 | 40086_at | 0.051 | 0.456 |
| 35435_s_at | 0.325 | 0.736 | 33405_at | 0.048 | 0.548 |
| 32511_at | 0.324 | 0.586 | 36165_at | 0.044 | 0.234 |
| 33229_at | 0.323 | 0.589 | 33819_at | 0.041 | 0.549 |
| 1512_at | 0.322 | 0.529 | 34378_at | 0.039 | 0.417 |
| 1197_at | 0.320 | 0.134 | 31993_f_at | 0.038 | 0.667 |
| 40191_s_at | 0.319 | 0.520 | 663_at | 0.037 | 0.531 |
| 34781_at | 0.318 | 0.509 | 34680_s_at | 0.037 | 0.521 |
| 855_at | 0.315 | 0.450 | 33421_s_at | 0.036 | 0.455 |
| 39351_at | 0.315 | 0.653 | 33892_at | 0.036 | 0.579 |
| 38400_at | 0.314 | 0.594 | 34826_at | 0.035 | 0.288 |
| 1074_at | 0.313 | 0.486 | 37029_at | 0.033 | 0.526 |
| 37732_at | 0.312 | 0.363 | 35253_at | 0.031 | 0.479 |
| 41737_at | 0.312 | 0.460 | 38371_at | 0.026 | 0.437 |
| 36544_at | 0.311 | 0.662 | 33850_at | 0.026 | 0.512 |
| 31508_at | 0.309 | 0.599 | 40487_at | 0.025 | 0.356 |
| 37536_at | 0.309 | 0.409 | 34366_g_at | 0.025 | 0.420 |
| 38046_at | 0.307 | 0.599 | 35751_at | 0.025 | 0.605 |
| 37025_at | 0.305 | 0.504 | 33848_r_at | 0.024 | 0.380 |
| 33451_s_at | 0.304 | 0.449 | 274_at | 0.023 | 0.510 |
| 2039_s_at | 0.304 | 0.723 | 36201_at | 0.021 | 0.234 |
| 34391_at | 0.304 | 0.464 | 40555_at | 0.021 | 0.480 |
| 37736_at | 0.303 | 0.647 | 39373_at | 0.019 | 0.345 |
| 31855_at | 0.303 | 0.274 | 35304_at | 0.017 | 0.511 |
| 218_at | 0.301 | 0.621 | 39762_at | 0.017 | 0.571 |
| 32506_at | 0.300 | 0.558 | 33255_at | 0.015 | 0.334 |
| 1394_at | 0.300 | 0.425 | 35258_f_at | 0.013 | 0.507 |
| 31907_at | 0.299 | 0.539 | 40988_at | 0.011 | 0.522 |
| 32563_at | 0.299 | 0.469 | 933_f_at | 0.011 | 0.604 |
| 41424_at | 0.299 | 0.588 | 1447_at | 0.010 | 0.467 |
| 33418_at | 0.299 | 0.553 | 37672_at | 0.009 | 0.518 |
| 39183_at | 0.298 | 0.463 | 38995_at | 0.006 | 0.114 |
| 40903_at | 0.293 | 0.483 | 890_at | 0.006 | 0.548 |

Note:
Absolute CC values are shown for expression levels analyzed in all 36 samples (R1) and in the 18 test samples only (R2).
CCs ≧0.5 are italicized and underlined.

### REFERENCES CITED

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method to determine if a patient is afflicted with ovarian cancer comprising:
a) obtaining a sample from the said patient;
b) determining the levels of gene expression of two or more of the genes listed in Table 9 in the sample from the patient;
c) comparing the levels of gene expression of the two or more genes determined in (b) to the levels of the same genes listed in Table 1;
d) determining the degree of similarity (DOS) between the levels of gene expression of the two or more genes determined in (c); and
e) determining from the DOS between the level of gene expression of the two or more genes the probability that the sample shows evidence of the presence of ovarian cancer in the patient.

2. The method of claim 1, wherein the levels of gene expression are determined for a subset of the genes listed in table comprising genes Nos. 1-28 in Table 9.

3. The method of claim 1, wherein the sample comprises cells obtained from the patient.

4. The method of claim 1, wherein the sample comprises cells removed from a solid tumor in the said patient.

5. The method of claim 1, wherein the sample comprises blood cells and serum drawn from the said patient.

6. The method of claim 1, wherein the sample comprises a body fluid drawn from the patient.

7. The method of claim 1, wherein the method of determining the level of gene expression comprises measuring the levels of protein expression product in the sample from the patient.

8. The method of claim 7, wherein the presence and level of the protein expression products are detected using a reagent which specifically binds with the proteins.

9. The method of claim 8, wherein the reagent is selected from the group consisting of an antibody, an antibody derivative and an antibody fragment.

10. The method of claim 1, wherein the levels of expression in the sample are assessed by measuring the levels in the sample of the transcribed polynucleotides of the two or more gene in Table 9.

11. The method of claim 10, wherein the transcribed polynucleotide is an mRNA.

12. The method of claim 10, wherein the transcribed polynucleotide is a cDNA.

13. The method of claim 10, wherein the step of detecting further comprises amplifying the transcribed polynucleotide.

14. The method of claim 1, wherein the method is performed ex vivo.

15. A method of treating a subject afflicted with ovarian cancer, the method comprising providing to cells of the subject an antisense oligonuceotide complimentary to one or more of the genes whose expression is up-regulated in ovarian cancer as shown in Table 6.

16. A method of inhibiting ovarian cancer in a subject at risk for developing ovarian cancer, the method comprising inhibiting expression of one or more of the genes shown in Table 6 to be up-regulated in ovarian cancer.

17. A kit for use in determining treatment strategy for a patient with suspected ovarian cancer comprising:
a) two or more antibodies able to recognize and bind to the polypeptide expression product of the two or more of the genes in Table 9;
b) a container suitable for containing the said antibodies and a sample of body fluid from the said individual wherein the antibody can contact the polypeptide expressed by the two or more genes shown in Table 9 if they are present;
c) means to detect the combination of the said antibodies with the polypeptides expressed by the two or more genes shown in Table 9; and
d) instructions for use and interpretation of the kit results.

18. A kit for use in determining the presence or absence of ovarian cancer in a patient comprising:
a) two or more polypeptides able to recognize and bind to the mRNA expression product of the hero or more genes shown in Table 9;
b) a container suitable for containing the said polynucleotides and a sample of body fluid from the said individual wherein the said polynucleotide can contact the mRNA, if it is present;
c) means to detect the levels of combination of the said polynucleotide with the mRNA from the two or more genes shown in Table 9; and
d) instructions for use and interpretation of the kit results.
